(19) United States
(12) Patent Application Publication
(10) Pub. No.: US 2019/0388341 A1
(43) Pub. Date: Dec. 26, 2019

(54) CHEWABLE GEL DOSAGE FORM AND ASSOCIATED METHODS

(71) Applicant: Santa Cruz Pharmaceuticals. Inc., Santa Cruz, CA (US)

(72) Inventors: Michael A. SIMPSON, Mooresville, NC (US); Benoit MINVILLE, Cowansville (CA); William BOST, Fairview Park, OH (US); Alain CARRIER, Saint-Hubert (CA)

(73) Assignee: Santa Cruz Pharmaceuticals. Inc., Santa Cruz, CA (US)

(21) Appl. No.: 16/372,177

(22) Filed: Apr. 1, 2019

Related U.S. Application Data
(60) Provisional application No. 62/770,659, filed on Nov. 21, 2018.
(60) Provisional application No. 62/690,320, filed on Jun. 26, 2018.

(51) Int. Cl. A61K 9/00; A61P 37/08; A61K 31/4545; A61K 47/26; A61K 47/36; A61K 47/12; A61K 9/06

(52) U.S. Cl. CPC A61K 9/0056; A61P 37/08; A61K 31/4545; A61K 9/06; A61K 47/36; A61K 47/12; A61K 47/26

(57) ABSTRACT

The invention provides a chewable gel dosage form that contains at least one active agent and a method of administering such chewable gel dosage form to subjects to treat one or more indications associated with the active agent(s). In one embodiment, the invention provides a method for the temporary relief of symptoms to due hay fever and upper respiratory allergies comprising administering to a subject in need thereof a chewable gel dosage form comprising a therapeutically effective amount of loratadine and a gelling agent, wherein therapeutic efficacy is achieved under fasted or fed conditions.

### CROSS-REFERENCE TO RELATED APPLICATIONS

This patent application claims the benefit of U.S. Provisional Application No. 62/690,320, filed Jun. 26, 2018, and U.S. Provisional Application No. 62/770,659, filed Nov. 21, 2018, which are hereby incorporated herein by reference in their entireties.

### BACKGROUND OF THE INVENTION

Oral solid unit dosage forms, such as tablets or liquid filled capsules, provide discreet doses of drugs, including prescription and over-the-counter drugs, but can present challenges for individuals who have trouble swallowing tablets. In addition, most people need a source of water or other liquid, which is not always readily available, to swallow tablets and capsules. While conventional liquid dosage are generally easier to consume than conventional solid forms, the consumption of liquid dosage forms can result in significant dosage variations, which are difficult to control especially for self-administered, over-the-counter products. All of these issues can adversely affect patient compliance with the specified treatment regimen thereby leading to suboptimal results.

Furthermore, conventional solid oral unit doses of drugs can exhibit undesirable pharmacokinetic properties, which may increase the likelihood of obtaining inconsistent or undesirable blood levels of active agent per unit dose. The oral administration of drugs thus presents challenges with respect to consistently providing the desired therapeutic effects. Accordingly, a need exists for oral dosage forms of drugs, including prescription and over-the counter drugs, which are easy to administer and consume, deliver precise dosages, improve patient compliance, and exhibit desirable pharmacokinetic properties. The present invention provides such dosage forms.

### BRIEF SUMMARY OF THE INVENTION

The present invention provides a chewable gel dosage form comprising an active agent, methods of administering such chewable gel dosage forms, and methods of producing such dosage forms. The active agent of the chewable gel dosage form of the invention may include, for example, an anti-inflammatory, an antirheumatic, an antipyretic, an antiemetic, an analgesic, an antiepileptic, an antipsychotic, an antidepressant, a hypnotic, an anti-ulceric, a prokinetic, an anti-asthmatic, an antiparkinsonic, a cardiovascular, a vasodilator, a urologic, a diuretic, an erectile dysfunction medication, a hypolipidemic, an anti-diabetic, an antihistaminic active ingredient, or a pharmaceutically acceptable salt, ester, hydrate or solvate thereof, and any suitable combination of two or more of such active agents. Examples of active agent include loratadine, diphenhydramine, desloratadine, phenylephrine, chlorpheniramine, dextromethorphan, doxylamine, guaifenesin, fexofenadine, docusate, pseudoephedrine, cetirizine, triprolidine, brompheniramine, ephedrine, ibuprofen, acetaminophen (paracetamol), ketoprofen, naproxen, piroxicam, meloxicam, leflunomide, ondansetron, granisetron, carbamazepine, lamotrigine, clozapine, olanzapine, risperidone, citalopram, paroxetine, sertraline, fluoxetine, fluvoxamine, zopiclon, zolpidem, cimetidine, ranitidine, omeprazole, metoclopramide, cisapride, domperidon, zafirlukast, montelukast, pramipexol, selegiline, doxazosin, terazosin, atenolol, bisoprolol, amlodipine, nifedipine, diltiazem, enalapril, captopril, ramipril, losartan, glyceroltrinitrate, alfuzosin, finasteride, pravastatin, atorvastatin, simvastatin, gemfibrozil, metformin, terfenadine, celecoxib, rifecoxib, rivastignine, astemizole, hydroxyzine, clemastine, local anesthestics, antiseptics, opioids, opioid derivatives, sildenafil, tadalafil, vardenafil, or a pharmaceutically acceptable salt, ester, hydrate or solvate thereof, and any suitable combination of two or more of such active agents.

In some embodiments, the present invention provides a chewable gel dosage form comprising loratadine, methods of administering such chewable gel dosage forms, and methods of producing such dosage forms. Loratadine, the active pharmaceutical ingredient in Claritin®, is a tricyclic antihistamine with selective peripheral Hi receptor antagonist activity. Loratadine reduces the effect of histamine in the body and is used is to treat sneezing, runny nose, watery eyes, hives, skin rash, itching, and other cold or allergy symptoms. Loratadine is presently marketed in various dosage forms for oral administration including, tablets, liquid filled capsules, and liquid suspensions.

In some embodiments, the invention is directed to chewable gel dosage form comprising loratadine and a gelling agent, which dosage form provides improved pharmacokinetic parameters following oral administration. The chewable gel dosage form of the invention comprising an antihistamine such as, e.g., loratadine is useful for administration to individuals, including both adults and children, to treat symptoms associated with hay fever, allergies, and other conditions that are potentially responsive to antihistamine treatment. In some embodiments, the chewable gel dosage form of the invention provides for the oral delivery of loratadine with the same dose accuracy of current oral solid unit doses, but without the difficulties associated with swallowing as required conventional solid dosage forms. The chewable gel dosage form of the invention comprising loratadine also reduces the likelihood of low loratadine blood levels relative to conventional solid dosage forms, thereby increasing the likelihood of achieving the desired clinical results.

In one aspect, the invention provides a method for the temporary relief of symptoms due to hay fever and upper respiratory allergies comprising orally administering to a subject in need thereof a chewable gel dosage form comprising about 10 mg loratadine and at least one gelling agent, to provide an AUC(0-t) and AUC(0-inf) of loratadine of from about 3 ng*hr/mL to about 25 ng*hr/mL.

In another aspect, the invention provides a method for the temporary relief of symptoms due to hay fever and upper respiratory allergies comprising orally administering to a subject in need thereof a chewable gel dosage form comprising about 10 mg loratadine and at least one gelling agent, to provide, under fasted conditions, an AUC(0-t) of loratadine of from about 7 ng*hr/mL to about 12 ng*hr/mL and an AUC(0-inf) of loratadine from about 8 ng*hr/mL to about 12 ng*hr/mL. In some embodiments, the chewable gel dosage form of the invention provides, under fasted conditions, an AUC(0-t) of loratadine of from about 7.343 ng*hr/mL to about 11.474 ng*hr/mL and an AUC(0-inf) of loratadine from about 7.555 ng*hr/mL to about 11.805 ng*hr/mL.

In a further aspect, the invention provides a method for the temporary relief of symptoms due to hay fever and upper respiratory allergies comprising orally administering to a subject in need thereof a chewable gel dosage form comprising about 10 mg loratadine and at least one gelling agent, to provide, under fed conditions, an AUC(0-t) of loratadine from about 11 ng*hr/mL to about 17 ng*hr/mL and an AUC(0-inf) of loratadine is from about 12 ng*hr/mL to about 18 ng*hr/mL. In some embodiments, the chewable gel dosage form of the invention provides, under fed conditions, an AUC(0-t) of loratadine from about 11.001 ng*hr/mL to about 17.189 ng*hr/mL and an AUC(0-inf) of loratadine is from about 11.764 ng*hr/mL to about 18.381 ng*hr/mL.

The invention also provides a method for the temporary relief of symptoms due to hay fever and upper respiratory allergies comprising orally administering to a subject in need thereof a chewable gel dosage form comprising about 10 mg loratadine and at least one gelling agent, to provide, under fasted conditions, an AUC(0-t) and an AUC(0-inf) of loratadine greater than about 5 ng*hr/mL. In some embodiments, the chewable gel dosage form of the invention provides, under fasted conditions, an AUC(0-t) of loratadine greater than about 4.951 ng*hr/mL and an AUC(0-inf) of loratadine greater than about 5.009 ng*hr/mL.

The invention additionally provides a method for the temporary relief of symptoms due to hay fever and upper respiratory allergies comprising orally administering to a subject in need thereof a chewable gel dosage form comprising about 10 mg loratadine and at least one gelling agent, to provide, under fed conditions, an AUC(0-t) of loratadine greater than about 8 ng*hr/mL and an AUC(0-inf) of loratadine greater than about 9 ng*hr/mL. In some embodiments, the chewable gel dosage form of the invention provides, under fed conditions, an AUC(0-t) of loratadine greater than about 8.190 ng*hr/mL and an AUC(0-inf) of loratadine greater than about 8.557 ng*hr/mL.

The invention further provides a method for the temporary relief of symptoms due to hay fever and upper respiratory allergies comprising orally administering to a subject in need thereof a chewable gel dosage form comprising about 10 mg loratadine and at least one gelling agent, to provide, under fasted conditions, an AUC(0-t) of loratadine from about 5 ng*hr/mL to about 17 ng*hr/mL and an AUC(0-inf) of loratadine from about 5 ng*hr/mL to about 18 ng*hr/mL. In some embodiments, the chewable gel dosage form of the invention provides, under fasted conditions, an AUC(0-t) of loratadine from about 4.951 ng*hr/mL to about 16.496 ng*hr/mL and an AUC(0-inf) of loratadine from about 5.009 ng*hr/mL to about 17.625 ng*hr/mL.

The invention moreover provides a method for the temporary relief of symptoms due to hay fever and upper respiratory allergies comprising orally administering to a subject in need thereof a chewable gel dosage form comprising about 10 mg loratadine and at least one gelling agent, to provide, under fed conditions, an AUC(0-t) of loratadine from about 8 ng*hr/mL to about 22 ng*hr/mL and an AUC(0-inf) of loratadine from 9 ng*hr/mL to about 22 ng*hr/mL. In some embodiments, the chewable gel dosage form of the invention provides, under fed conditions, an AUC(0-t) of loratadine from about 8.190 ng*hr/mL to about 22.057 ng*hr/mL and an AUC(0-inf) of loratadine from 8.557 ng*hr/mL to about 21.868 ng*hr/mL.

The invention also includes a method for the temporary relief of symptoms due to hay fever and upper respiratory allergies comprising orally administering to a subject in need thereof a chewable gel dosage form comprising about 10 mg loratadine and at least one gelling agent, to provide, under fasted conditions, a first quartile AUC(0-t) or AUC(0-inf) of loratadine of from about 1.5 to about 2.3 fold of the first quartile AUC(0-t) or AUC(0-inf) of loratadine provided by a conventional liquid capsule comprising about 10 mg loratadine.

The invention additionally includes a method for the temporary relief of symptoms due to hay fever and upper respiratory allergies comprising administering to a subject in need thereof a chewable gel dosage form comprising about 10 mg loratadine and at least one gelling agent, to provide, under fasted conditions, a first quartile Cₘₐₓ of from about 1.2 to about 1.7 fold of the first quartile AUC(0-t) or AUC(0-inf) of loratadine provided by a conventional liquid capsule comprising about 10 mg loratadine.

The invention further includes a method for the temporary relief of symptoms due to hay fever and upper respiratory allergies comprising orally administering to a subject in need thereof a chewable gel dosage form comprising about 10 mg loratadine and at least one gelling agent, to provide, under fed conditions, a first quartile AUC(0-t) or AUC(0-inf) of loratadine of from about 1.2 to about 1.6 fold of the first quartile AUC(0-t) or AUC(0-inf) of loratadine provided by a conventional liquid capsule comprising about 10 mg loratadine.

The invention moreover includes a method for the temporary relief of symptoms due to hay fever and upper respiratory allergies comprising orally administering to a subject in need thereof a chewable gel dosage form comprising about 10 mg loratadine and at least one gelling agent, to provide, under fed conditions, a first quartile Cₘₐₓ of loratadine of from about 1.2 to about 1.7 fold of the first quartile Cₘₐₓ of loratadine provided by a conventional liquid capsule comprising about 10 mg loratadine, when administered orally.

In one aspect, the invention provides a chewable gel dosage form comprising about 10 mg loratadine and at least one gelling agent, which provides, under either fed or fasted conditions, an AUC(0-t) and AUC(0-inf) of loratadine from about 3 ng*hr/mL to about 25 ng*hr/mL, when administered orally.

In another aspect, the invention provides a chewable gel dosage form comprising about 10 mg loratadine and at least one gelling agent, which provides, under fasted conditions, an AUC(0-t) of loratadine from about 7 ng*hr/mL to about 11 ng*hr/mL and an AUC(0-inf) of loratadine from about 8 ng*hr/mL to about 12 ng*hr/mL, when administered orally. In some embodiments, the chewable gel dosage form of the invention provides, under fasted conditions, an AUC(0-t) of loratadine from about 7.343 ng*hr/mL to about 11.474 ng*hr/mL and an AUC(0-inf) of loratadine from about 7.555 ng*hr/mL to about 11.805 ng*hr/mL, when administered orally.

In yet another aspect, the invention includes a chewable gel dosage form comprising about 10 mg loratadine and at least one gelling agent, which provides, under fed conditions, an AUC(0-t) of loratadine from about 11 ng*hr/mL to about 17 ng*hr/mL and an AUC(0-inf) of loratadine is from about 12 ng*hr/mL to about 18 ng*hr/mL, when administered orally. In some embodiments, the chewable gel dosage form of the invention provides, under fed conditions, an AUC(0-t) of loratadine from about 11.001 ng*hr/mL to about 17.189 ng*hr/mL and an AUC(0-inf) of loratadine is from about 11.764 ng*hr/mL to about 18.381 ng*hr/mL, when administered orally.

In a further aspect, the invention includes a chewable gel dosage form comprising about 10 mg loratadine and at least one gelling agent, which provides, under fasted conditions, an AUC(0-t) and an AUC(0-inf) of loratadine greater than about 5 ng*hr/mL, when administered orally. In some embodiments, the chewable gel dosage form of the invention provides, under fasted conditions, an AUC(0-t) of loratadine greater than about 4.951 ng*hr/mL and an AUC(0-inf) of loratadine greater than about 5.009 ng*hr/mL, when administered orally.

The invention also provides a chewable gel dosage form comprising about 10 mg loratadine and at least one gelling agent, which provides, under fed conditions, an AUC(0-t) of loratadine greater than about 8 ng*hr/mL and an AUC(0-inf) of loratadine greater than about 9 ng*hr/mL, when administered orally. In some embodiments, the chewable gel dosage form of the invention provides, under fed conditions, an AUC(0-t) of loratadine greater than about 8.190 ng*hr/mL and an AUC(0-inf) of loratadine greater than about 8.557 ng*hr/mL, when administered orally.

The invention additionally provides a chewable gel dosage form comprising about 10 mg loratadine and at least one gelling agent, which provides, under fasted conditions, an AUC(0-t) of loratadine from about 5 ng*hr/mL to about 17 ng*hr/mL and an AUC(0-inf) of loratadine from about 5 ng*hr/mL to about 18 ng*hr/mL, when administered orally. In some embodiments, the chewable gel dosage form of the invention provides, under fasted conditions, an AUC(0-t) of loratadine from about 4.951 ng*hr/mL to about 16.496 ng*hr/mL and an AUC(0-inf) of loratadine from about 5.009 ng*hr/mL to about 17.625 ng*hr/mL, when administered orally.

The invention further provides a chewable gel dosage form comprising about 10 mg loratadine and at least one gelling agent, which provides, under fed conditions, an AUC(0-t) of loratadine from about 8 ng*hr/mL to about 22 ng*hr/mL and an AUC(0-inf) of loratadine from 9 ng*hr/mL to about 22 ng*hr/mL, when administered orally. In some embodiments, the chewable gel dosage form of the invention provides, under fed conditions, an AUC(0-t) of loratadine from about 8.190 ng*hr/mL to about 22.057 ng*hr/mL and an AUC(0-inf) of loratadine from 8.557 ng*hr/mL to about 21.868 ng*hr/mL, when administered orally.

The invention moreover provides a chewable gel dosage form comprising about 10 mg loratadine and at least one gelling agent, which provides, under fasted conditions, a first quartile AUC(0-t) or AUC(0-inf) of loratadine of from about 1.5 to about 2.3 fold of the AUC(0-t) and AUC(0-inf) of loratadine provided by a conventional liquid capsule comprising about 10 mg loratadine, when administered orally.

The invention is also drawn to a chewable gel dosage form comprising about 10 mg loratadine and at least one gelling agent, which provides, under fasted conditions, a first quartile Cₘₐₓ of loratadine of from about 1.2 to about 1.7 fold of the Cₘₐₓ of loratadine provided by a conventional liquid capsule comprising about 10 mg loratadine, when administered orally.

The invention is further drawn to a chewable gel dosage form comprising about 10 mg loratadine and at least one gelling agent, which provides, under fed conditions, a first quartile AUC(0-t) or AUC(0-inf) of loratadine of from about 1.2 to about 1.6 fold of the AUC(0-t) and AUC(0-inf) of loratadine provided by a conventional liquid capsule comprising about 10 mg loratadine, when administered orally.

The invention is moreover drawn to a chewable gel dosage form comprising about 10 mg loratadine and at least one gelling agent, which provides, under fed conditions, a first quartile Cₘₐₓ of loratadine of from about 1.2 to about 1.7 fold of the Cₘₐₓ of loratadine provided by a conventional liquid capsule comprising about 10 mg loratadine, when administered orally.

These and other embodiments, characteristics, and advantages of the invention are apparent from the descriptions and examples that follow.

### BRIEF DESCRIPTION OF THE DRAWINGS

FIG. 1 is a linear plot of the mean plasma concentration of loratadine in ng/mL versus time elapsed from administration under fed conditions of (1) a chewable gel dosage form containing 10 mg loratadine (test product) and (2) Claritin® 10 mg loratadine Liqui-Gels® capsule (reference product).

FIG. 2 is a semi-log plot of the mean plasma concentration of loratadine in ng/mL versus time elapsed from administration under fed conditions of (1) a chewable gel dosage form containing 10 mg loratadine (test product) and (2) Claritin® 10 mg loratadine Liqui-Gels® capsule (reference product).

FIG. 3 is a linear plot of the mean plasma concentration of desloratadine in ng/mL versus time elapsed from administration under fed conditions of (1) a chewable gel dosage form containing 10 mg loratadine (test product) and (2) Claritin® 10 mg loratadine Liqui-Gels® capsule (reference product).

FIG. 4 is a semi-log plot of the mean plasma concentration of loratadine in ng/mL versus time elapsed from administration under fed conditions of (1) a chewable gel dosage form containing 10 mg loratadine (test product) and (2) Claritin® 10 mg loratadine Liqui-Gels® capsule (reference product).

FIG. 5 is a linear plot of the mean plasma concentration of loratadine in ng/mL versus time elapsed from administration under fasted conditions of (1) a chewable gel dosage form containing 10 mg loratadine (test product) and (2) Claritin® 10 mg loratadine Liqui-Gels® capsule (reference product).

FIG. 6 is a semi-log plot of the mean plasma concentration of loratadine in ng/mL versus time elapsed from administration under fasted conditions of (1) a chewable gel dosage form containing 10 mg loratadine (test product) and (2) Claritin® 10 mg loratadine Liqui-Gels® capsule (reference product).

FIG. 7 is a linear plot of the mean plasma concentration of desloratadine in ng/mL versus time elapsed from administration under fasted conditions of (1) a chewable gel dosage form containing 10 mg loratadine (test product) and (2) Claritin® 10 mg loratadine Liqui-Gels® capsule (reference product).

FIG. 8 is a semi-log plot of the mean plasma concentration of desloratadine in ng/mL versus time elapsed from administration under fasted conditions of (1) a chewable gel dosage form containing 10 mg loratadine (test product) and (2) Claritin® 10 mg loratadine Liqui-Gels® capsule (reference product).

FIG. 9 is a plot of the mean plasma concentration of loratadine in pg/mL versus time elapsed (on a linear scale) from administration under fasted conditions of (1) a chewable gel dosage form containing 10 mg loratadine (test product), (2) Claritin® 10 mg loratadine tablet (reference product 1), (3) Claritin® 10 mL (5 mg/5 mL) loratadine oral solution (reference product 2), and (4) Claritin® 10 mg loratadine Liqui-Gels® capsule (reference product 3).

FIG. 10 is a plot of the mean plasma concentration of loratadine in pg/mL versus time elapsed (on a semi-logarithmic scale) from administration under fasted conditions of (1) a chewable gel dosage form containing 10 mg loratadine (test product), (2) Claritin® 10 mg loratadine tablet (reference product 1), (3) Claritin® 10 mL (5 mg/5 mL) loratadine oral solution (reference product 2), and (4) Claritin® 10 mg loratadine Liqui-Gels® capsule (reference product 3).

FIG. 11 is a plot of the mean plasma concentration of desloratadine in pg/mL versus time elapsed (on a linear scale) from administration under fasted conditions of (1) a chewable gel dosage form containing 10 mg loratadine (test product), (2) Claritin® 10 mg loratadine tablet (reference product 1), (3) Claritin® 10 mL (5 mg/5 mL) loratadine oral solution (reference product 2), and (4) Claritin® 10 mg loratadine Liqui-Gels® capsule (reference product 3).

FIG. 12 is a plot of the mean desloratadine plasma concentration in pg/mL versus time elapsed (on a semi-logarithmic scale) from administration under fasted conditions of (1) a chewable gel dosage form containing 10 mg loratadine (test product), (2) Claritin® 10 mg loratadine tablet (reference product 1), (3) Claritin® 10 mL (5 mg/5 mL) loratadine oral solution (reference product 2), and (4) Claritin® 10 mg loratadine Liqui-Gels® capsule (reference product 3).

### DETAILED DESCRIPTION OF THE INVENTION

The chewable gel dosage form of the invention (also referred to herein the “dosage form”) is a semi-solid product that is intended to be chewed by a subject such that it is broken up into smaller parts within the oral cavity and then easily swallowed. The chewable gel dosage form has a sufficiently high viscosity that it is not pourable and further does not flow or conform to its container at room temperature. Typically, the chewable gel dosage form does not flow at low shear stress and generally exhibits plastic flow behavior. In general, the consistency of the chewable gel dosage form is the same as or similar to gelatin-based or pectin-based candy products such as, for example, gummy bears and pectin jellies.

The dosage form can have any size and shape such that it can be administered orally and chewed by a subject. The subject should be able to readily break apart the dosage form by chewing and swallowing the dosage form without the need for an external source of liquid. The chewable gel dosage form of the present invention is intended to be chewed prior to swallowing, and may be packaged with instructions recommending that the dosage form be chewed before swallowing. One of ordinary skill in the art will appreciate that chewing of the chewable gel dosage form of the invention, e.g., as instructed by a suitable package insert, may result in deformation and/or dissolution of at least a portion of the dosage form without necessarily breaking apart of the dosage form. It is also possible that chewing of the chewable gel dosage form of the invention may result in the breaking apart of at least a portion of the dosage form. In some instances, chewing of the chewable gel dosage form of the invention may result in deformation, dissolution, and/or breaking apart of some or all of the dosage form. The extent to which the dosage form is deformed, dissolved, and/or broken apart may depend on how the instructions are written, how the instructions are interpreted by the subject, the amount of bite force used by the subject, the physical condition of the subject, the physical and/or chemical environment of the subject's mouth, and other factors that may impact how and to what extent the dosage form is processed in the subject's mouth before swallowing.

The chewable gel dosage form of the invention may have any suitable shape, including, for example, spheres, ovals, cylinders, rectangular boxes, cubes, fruit shapes (e.g., the shape of berries, citrus fruits, segments of citrus fruits, etc.), plant shapes, animal shapes, worms, or any combination thereof. In some embodiments, the dosage form of the invention has a length of about 1 cm to about 5 cm, width of about 1 cm to about 5 cm and a height of about 1 cm to about 5 cm. Suitable shapes include, for example, ovals, spheres, cylinders, rectangular boxes and cubes. The dosage form may be formed into unique shapes and figures including, for example, animals for administration to children (e.g., under the age of 13 or ages 2-6) and/or adults.

In some embodiments, each individual dosage form has a total weight of at least about 100 mg. For example, the chewable gel dosage form of the invention may have a total weight of from about 100 mg to about 10,000 mg, from about 100 mg to about 7,500 mg, from about 100 mg to about 5,000 mg, from about 100 mg to about 4,000 mg, from about 100 mg to about 3,000 mg, from about 100 mg to about 2,500 mg, from about 100 mg to about 2,000 mg, from about 100 mg to about 1,500 mg, from about 100 mg to about 1,000 mg, from about 100 mg to about 750 mg, from about 100 mg to about 500 mg, from about 100 mg to about 250 mg, from about 250 mg to about 10,000 mg, from about 250 mg to about 7,500 mg, from about 250 mg to about 5,000 mg, from about 250 mg to about 4,000 mg, from about 250 mg to about 3,000 mg, from about 250 mg to about 2,500 mg, from about 250 mg to about 2,000 mg, from about 250 mg to about 1,500 mg, from about 250 mg to about 1,000 mg, from about 250 mg to about 750 mg, from about 250 mg to about 500 mg, from about 500 mg to about 10,000 mg, from about 500 mg to about 7,500 mg, from about 500 mg to about 5,000 mg, from about 500 mg to about 4,000 mg, from about 500 mg to about 3,000 mg, from about 500 mg to about 2,500 mg, from about 500 mg to about 2,000 mg, from about 500 mg to about 1,500 mg, from about 500 mg to about 1,000 mg, from about 500 mg to about 750 mg, from about 750 mg to about 10,000 mg, from about 750 mg to about 7,500 mg, from about 750 mg to about 5,000 mg, from about 750 mg to about 4,000 mg, from about 750 mg to about 3,000 mg, from about 750 mg to about 2,500 mg, from about 750 mg to about 2,000 mg, from about 7 50 mg to about 1,500 mg, from about 750 mg to about 1,000 mg, from about 1,000 mg to about 10,000 mg, from about 1,000 mg to about 7,500 mg, from about 1,000 mg to about 5,000 mg, from about 1,000 mg to about 4,000 mg, from about 1,000 mg to about 3,000 mg, from about 1,000 mg to about 2,500 mg, from about 1,000 mg to about 2,000 mg, from about 2,000 mg to about 10,000 mg, from about 2,000 mg to about 7,500 mg, from about 2,000 mg to about 5,000 mg, from about 2,000 mg to about 4,000 mg, from about 2,000 mg to about 3-000 mg, from about 2,000 mg to about 2,500 mg, from about 2,500 mg to about 10,000 mg, from about 2,500 mg to about 7,500 mg, from about 2,500 mg to about 5,000 mg, from about 2,500 mg to about 4,000 mg, from about 2,500 mg to about 3,000 mg, from about 3,000 mg to about 10,000 mg, from about 3,000 mg to about 7,500 mg, from about 3,000 mg to about 5,000 mg, from about 3,000 mg to about 4,000 mg, from about 4,000 mg to about 10,000 mg, from about 4,000 mg to about 7,500 mg, from about 4,000 mg to about 5,000 mg, from about 5,000 mg to about 10,000 mg, from about 5,000 mg to about 7,500 mg, or from about 7,500 mg to about 10,000 mg. In some embodiments, each dosage form has a total weight of from about 1 g to about 20 g. For example, each dosage form may have a total weight of from about 1 g to about 15 g. In some embodiments, each dosage form has a total weight of from about 1 g to about 10 g. For example, each dosage form can have a total weight of about 1 g to about 1.5 g, about 1.5 g to about 2 g, about 2 g to about 2.5 g, about 2.5 g to about 3 g, about 3.5 g to about 4 g, about 4 g to about 4.5 g, about 4.5 g to about 5 g, about 5 g to about 5.5 g, about 5.5 g to about 6 g, about 6 g to about 6.5 g, about 6.5 g to about 7 g, about 7 g to about 7.5 g, about 7.5 g to about 8 g, about 8 g to about 8.5 g, about 8.5 g to about 9 g, about 9 g to about 9.5 g, and about 9.5 g to about 10 g. In some embodiments, each dosage form has a total weight of about 1 g to about 5 g. For example, in some embodiments, each dosage form has a total weight of about 5 g, while in other embodiments, each dosage form has a total weight of about 2 g.

The active agent (or active pharmaceutical ingredient) of the chewable gel dosage form of the invention may include, for example, an anti-inflammatory, an antirheumatic, an antipyretic, an antiemetic, an analgesic, an antiepileptic, an antipsychotic, an antidepressant, a hypnotic, an anti-ulceric, a prokinetic, an anti-asthmatic, an antiparkinsonic, a cardiovascular, a vasodilator, a urologic, a diuretic, an erectile dysfunction medication, a hypolipidemic, an anti-diabetic, an antihistaminic active ingredient, or a pharmaceutically acceptable salt, ester, hydrate or solvate thereof, and any suitable combination of two or more of such active agents. Examples of the active agent may include, without limitation, loratadine, diphenhydramine, desloratadine, phenylephrine, chlorpheniramine, dextromethorphan, doxylamine, guaifenesin, fexofenadine, docusate, pseudoephedrine, cetirizine, triprolidine, brompheniramine, ephedrine, ibuprofen, acetaminophen (paracetamol), ketoprofen, naproxen, piroxicam, meloxicam, leflunomide, ondansetron, granisetron, carbamazepine, lamotrigine, clozapine, olanzapine, risperidone, citalopram, paroxetine, sertraline, fluoxetine, fluvoxamine, zopiclon, zolpidem, cimetidine, ranitidine, omeprazole, metoclopramide, cisapride, domperidon, zafirlukast, montelukast, pramipexol, selegiline, doxazosin, terazosin, atenolol, bisoprolol, amlodipine, nifedipine, diltiazem, enalapril, captopril, ramipril, losartan, glyceroltrinitrate, alfuzosin, finasteride, pravastatin, atorvastatin, simvastatin, gemfibrozil, metformin, terfenadine, celecoxib, rifecoxib, rivastignine, astemizole, hydroxyzine, clemastine, local anesthestics, antiseptics, opioids (e.g., morphine, etc.), opioid derivatives (e.g., morphine derivatives), sildenafil, tadalafil, vardenafil, as well as any pharmaceutically acceptable salts, esters, hydrates or solvates thereof. In some embodiments, the API is one or more of loratadine, diphenhydramine, desloratadine, phenylephrine, chlorpheniramine, dextromethorphan, doxylamine, guaifenesin, fexofenadine, docusate, pseudoephedrine, cetirizine, triprolidine, brompheniramine, or a pharmaceutically acceptable salt, ester, hydrate, or solvate thereof, and any suitable combination of two or more of such active agents. The chewable gel dosage form of the invention preferably contains a therapeutically effective amount of the active agent.

The amount of active agent in the chewable gel dosage form of the invention can range, for example, from about 0.01% w/w to about 50% w/w (i.e., % active agent based on the total weight of the dosage form). In some embodiments, the amount or concentration of active agent in the chewable gel dosage form of the invention is less than about 50% w/w, less than about 40% w/w, less than about 35% w/w, less than about 30% w/w, less than about 25% w/w, less than about 20% w/w, less than about 15% w/w, less than about 10% w/w, less than about 9% w/w, less than about 8% w/w, less than about 7% w/w, less than about 6% w/w, less than about 5% w/w, less than about 4% w/w, less than about 3% w/w, less than about 2% w/w, less than about 1% w/w, less than about 0.5% w/w, less than about 0.1% w/w, less than about 0.05% w/w, or less than about 0.01% w/w.

In some embodiments, the amount of active agent in the chewable gel dosage form of the invention is greater than about 0.01% w/w, greater than about 0.05% w/w, greater than about 0.1% w/w, greater than about 0.5% w/w, greater than about 1% w/w, greater than about 2% w/w, greater than about 3% w/w, greater than about 4% w/w, greater than about 5% w/w, greater than about 6% w/w, greater than about 7% w/w, greater than about 8% w/w, greater than about 9% w/w, greater than about 10% w/w, greater than about 15% w/w, greater than about 20% w/w, greater than about 25% w/w, greater than about 30% w/w, greater than about 35% w/w, greater than about 40% w/w, or greater than about 45% w/w.

In some embodiments, the amount of active agent in the chewable gel dosage form of the invention has an upper limit of about 50% w/w, 45% w/w, 40% w/w, 35% w/w, 30% w/w, 25% w/w, 20% w/w, 15% w/w, 10% w/w, 9% w/w, 8% w/w, 7% w/w, 6% w/w, 5% w/w, 4% w/w, 3% w/w, 2% w/w, 1 w/w, 0.5% w/w, or 0.1% w/w, and an independently selected lower limit of about 0.01% w/w, 0.05% w/w, 0.1% w/w/, 0.5% w/w, 1% w/w, 2% w/w, 3% w/w, 4% w/w, 5% w/w, 6% w/w, 7% w/w, 8% w/w, 9% w/w, 10% w/w, 15% w/w, 20% w/w, 25% w/w, 30% w/w, 35% w/w, 40% w/w, or 45% w/w, where the lower limit is less than the upper limit.

In some embodiments, the amount of active agent in the chewable gel dosage form of the invention is about 0.01% w/w, about 0.05% w/w, about 0.1% w/w, about 0.25% w/w, about 0.5% w/w, about 0.75% w/w, about 1 w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, or about 50% w/w.

In some embodiments, the amount of active agent in the chewable gel dosage form of the invention can range from about 0.1 mg/unit dose to about 500 mg/unit dose. For example, the amount of active agent in the chewable gel dosage form of the invention can be less than about 500 mg/unit dose, less than about 450 mg/unit dose, less than about 400 mg/unit dose, less than about 350 mg/unit dose, less than about 300 mg/unit dose, less than about 250 mg/unit dose, less than about 200 mg/unit dose, less than about 150 mg/unit dose, less than about 100 mg/unit dose, less than about 75 mg/unit dose, less than about 50 mg/unit dose, less than about 25 mg/unit dose, less than about 10 mg/unit dose, less than about 9 mg/unit dose, less than about 8 mg/unit dose, less than about 7 mg/unit dose, less than about 6 mg/unit dose, less than about 5 mg/unit dose, less than about 4 mg/unit dose, less than about 3 mg/unit dose, less than about 2 mg/unit dose, less than about 1 mg/unit dose, less than about 0.75 mg/unit dose, or less than about 0.25 mg/unit dose.

In some embodiments, the amount of active agent in the chewable gel dosage form of the invention can be greater than about 0.1 mg/unit dose, greater than about 0.25 mg/unit dose, greater than about 0.75 mg/unit dose, greater than about 1 mg/unit dose, greater than about 2 mg/unit dose, greater than about 3 mg/unit dose, greater than about 4 mg/unit dose, greater than about 5 mg/unit dose, greater than about 6 mg/unit dose, greater than about 7 mg/unit dose, greater than about 8 mg/unit dose, greater than about 9 mg/unit dose, greater than about 10 mg/unit dose, greater than about 25 mg/unit dose, greater than about 50 mg/unit dose, greater than about 75 mg/unit dose, greater than about 100 mg/unit dose, greater than about 150 mg/unit dose, greater than about 200 mg/unit dose, greater than about 250 mg/unit dose, greater than about 300 mg/unit dose, greater than about 350 mg/unit dose, greater than about 400 mg/unit dose, or greater than about 450 mg/unit dose.

In some embodiments, the amount of active agent in the chewable gel dosage form of the invention may have an upper limit of about 500 mg/unit dose, 450 mg/unit dose, 400 mg/unit dose, 350 mg/unit dose, 300 mg/unit dose, 250 mg/unit dose, 200 mg/unit dose, 150 mg/unit dose, 100 mg/unit dose, 75 mg/unit dose, 50 mg/unit dose, 25 mg/unit dose, 10 mg/unit dose, 9 mg/unit dose, 8 mg/unit dose, 7 mg/unit dose, 6 mg/unit dose, 5 mg/unit dose, 4 mg/unit dose, 3 mg/unit dose, 2 mg/unit dose, 1 mg/unit dose, 0.75 mg/unit dose, or 0.25 mg/unit dose, and an independently selected lower limit of about 0.1 mg/unit dose, 0.25 mg/unit dose, 0.75 mg/unit dose, 1 mg/unit dose, 2 mg/unit dose, 3 mg/unit dose, 4 mg/unit dose, 5 mg/unit dose, 6 mg/unit dose, 7 mg/unit dose, 8 mg/unit dose, 9 mg/unit dose, 10 mg/unit dose, 25 mg/unit dose, 50 mg/unit dose, 75 mg/unit dose, 100 mg/unit dose, 150 mg/unit dose, 200 mg/unit dose, 250 mg/unit dose, 300 mg/unit dose, 350 mg/unit dose, 400 mg/unit dose, or 450 mg/unit dose, where the lower limit is less than the upper limit. For example, the amount of active agent in the chewable gel dosage form of the invention may be about 0.1 mg/unit dose, about 0.25 mg/unit dose, about 0.75 mg/unit dose, about 1 mg/unit dose, about 2 mg/unit dose, about 3 mg/unit dose, about 4 mg/unit dose, about 5 mg/unit dose, about 6 mg/unit dose, about 7 mg/unit dose, about 8 mg/unit dose, about 9 mg/unit dose, about 10 mg/unit dose, about 25 mg/unit dose, about 50 mg/unit dose, about 75 mg/unit dose, about 100 mg/unit dose, about 150 mg/unit dose, about 200 mg/unit dose, about 250 mg/unit dose, about 300 mg/unit dose, about 350 mg/unit dose, about 400 mg/unit dose, about 450 mg/unit dose, or about 500 mg/unit dose.

In some embodiments, the active agent is diphenhydramine, or a pharmaceutically acceptable salt, ester, hydrate, or solvate thereof (e.g., diphenhydramine HCl, diphenhydramine citrate, etc.). The amount of diphenhydramine, or a pharmaceutically acceptable salt, ester, hydrate, or solvate thereof in the chewable gel dosage form of the invention may include any suitable amount or concentration sufficient to achieve the desired efficacy with respect to one or more therapeutic indications associated with the active agent.

In some embodiments, the active agent is desloratadine, or a pharmaceutically acceptable salt, ester, hydrate, or solvate thereof. The amount of desloratadine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is phenylephrine, or a pharmaceutically acceptable salt, ester, hydrate, or solvate thereof (e.g., phenylephrine HCl, phenylephrine bitartrate, etc.). The amount of phenylephrine, or a pharmaceutically acceptable salt, ester, hydrate, or solvate thereof included in the chewable gel dosage form of the invention may include any amount or concentration sufficient to achieve the desired efficacy with respect to one or more therapeutic indications associated with the active agent.

In some embodiments, the active agent is chlorpheniramine, or a pharmaceutically acceptable salt, ester, hydrate, or solvate thereof (e.g., chlorpheniramine maleate, etc.). The amount of chlorpheniramine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is dextromethorphan, or a pharmaceutically acceptable salt, ester, hydrate, or solvate thereof (e.g., dextromethorphan HBr, etc.). The amount of dextromethorphan, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is doxylamine, or a pharmaceutically acceptable alt, ester, hydrate, or solvate thereof (e.g., doxylamine succinate, etc.). The amount of doxylamine, or a pharmaceutically acceptable alt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is guaifenesin, or a pharmaceutically acceptable salt, ester, hydrate, or solvate thereof. The amount of guaifenesin, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is fexofenadine, or a pharmaceutically acceptable salt, ester, hydrate, or solvate thereof (e.g., fexofenadine HCl, etc.). The amount of fexofenadine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is docusate, or a pharmaceutically acceptable salt, ester, hydrate, or solvate thereof (e.g., docusate sodium, etc.). The amount of docusate, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is pseudoephedrine, or a pharmaceutically acceptable salt, ester, hydrate, or solvate thereof (e.g., pseudoephedrine HCl, pseudoephedrine sulfate, etc.). The amount of pseudoephedrine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is cetirizine, or a pharmaceutically acceptable salt, ester, hydrate, or solvate thereof (e.g., cetirizine HCl, etc.). The amount of cetirizine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is triprolidine, or a pharmaceutically acceptable salt, ester, hydrate, or solvate thereof (e.g., triprolidine HCl, etc.). The amount of triprolidine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active agent is brompheniramine, or a pharmaceutically acceptable salt, ester, hydrate, or solvate thereof (e.g., brompheniramine maleate, etc.). The amount of brompheniramine, or a pharmaceutically acceptable salt, ester, hydrate, or solvate thereof included in the chewable gel dosage form of the invention may include any suitable amount or concentration sufficient to achieve the desired efficacy with respect to one or more therapeutic indications associated with the active agent.

In some embodiments, the active pharmaceutical ingredient useful in the invention is loratadine, or a pharmaceutically acceptable salt, ester, hydrate, or solvate thereof. Loratadine has the following chemical structure:

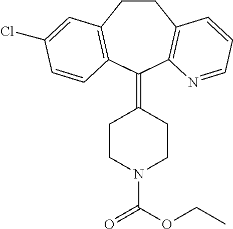 [SMILES: CCOC(=O)N1CCC(=C2c3ccc(Cl)cc3CCc3cccnc32)CC1]

The amount of loratadine, or a pharmaceutically acceptable salt, ester, hydrate, or solvate thereof included in the chewable gel dosage form of the invention may include any amount or concentration sufficient to achieve the desired efficacy with respect to one or therapeutic indications associated with the active agent. In some embodiments, the chewable gel dosage form of the invention contains loratadine, or a pharmaceutically acceptable salt, ester, hydrate, or solvate thereof, in an amount that ranges from about 0.01% w/w to about 50% w/w. In some embodiments, the chewable gel product contains loratadine, or a pharmaceutically acceptable salt, ester, hydrate, or solvate thereof, in an amount that is less than about 50% w/w, less than about 40% w/w, less than about 35% w/w, less than about 30% w/w, less than about 25% w/w, less than about 20% w/w, less than about 15% w/w, less than about 10% w/w, less than about 9% w/w, less than about 8% w/w, less than about 7% w/w, less than about 6% w/w, less than about 5% w/w, less than about 4% w/w, less than about 3% w/w, less than about 2% w/w, less than about 1% w/w, less than about 0.5% w/w, less than about 0.1% w/w, less than about 0.05% w/w, or less than about 0.01% w/w.

In some embodiments, the chewable gel dosage form of the invention contains loratadine, or a pharmaceutically acceptable salt, ester, hydrate, or solvate thereof, in an amount that is greater than about 0.01% w/w, greater than about 0.05% w/w, greater than about 0.1% w/w, greater than about 0.5% w/w, greater than about 1% w/w, greater than about 2% w/w, greater than about 3% w/w, greater than about 4% w/w, greater than about 5% w/w, greater than about 6% w/w, greater than about 7% w/w, greater than about 8% w/w, greater than about 9% w/w, greater than about 10% w/w, greater than about 15% w/w, greater than about 20% w/w, greater than about 25% w/w, greater than about 30% w/w, greater than about 35% w/w, greater than about 40% w/w, or greater than about 45% w/w.

In some embodiments, the chewable gel dosage form of the invention contains loratadine, or a pharmaceutically acceptable salt, ester, hydrate, or solvate thereof, in an amount that has a range of % w/w having an upper limit of about 50% w/w, 45% w/w, 40% w/w, 35% w/w, 30% w/w, 25% w/w, 20% w/w, 15% w/w, 10% w/w, 9% w/w, 8% w/w, 7% w/w, 6% w/w, 5% w/w, 4% w/w, 3% w/w, 2% w/w, 1% w/w, or 0.5% w/w, and an independently selected lower limit of about 0.01% w/w, 0.05% w/w, 0.1% w/w, 0.5% w/w, 1% w/w, 2% w/w, 3% w/w, 4% w/w, 5% w/w, 6% w/w, 7% w/w, 8% w/w, 9% w/w, 10% w/w, 15% w/w, 20% w/w, 25% w/w, 30% w/w, 35% w/w, 40% w/w, or 45% w/w, where the lower limit is less than the upper limit.

In some embodiments, the chewable gel dosage form of the invention contains loratadine, or a pharmaceutically acceptable salt, ester, hydrate, or solvate thereof, in an amount of at about 0.01% w/w, about 0.05% w/w, about 0.1% w/w, about 0.2% w/w, about 0.3% w/w, about 0.4% w/w, about 0.46% w/w, about 0.5% w/w, about 0.6% w/w, about 0.7% w/w, about 0.8% w/w, about 0.9% w/w, about 1% w/w, about 2% w/w, about 3% w/w, or about 4% w/w. In some embodiments, the chewable gel dosage form of the invention contains loratadine, or a pharmaceutically acceptable salt, ester, hydrate, or solvate thereof, in an amount that is about 0.46% w/w, about 0.5% w/w, or about 2% w/w.

In some embodiments, the chewable gel dosage form of the invention contains loratadine, or a pharmaceutically acceptable salt, ester, hydrate, or solvate thereof, in an amount that ranges from about 1 mg/unit dose to about 50 mg/unit dose. For example, the chewable gel dosage form of the invention may contain loratadine, or a pharmaceutically acceptable salt, ester, hydrate, or solvate thereof, in an amount that is less than about 50 mg/unit dose, less than about 40 mg/unit dose, less than about 30 mg/unit dose, less than about 25 mg/unit dose, less than about 20 mg/unit dose, less than about 15 mg/unit dose, less than about 10 mg/unit dose, less than about 9 mg/unit dose, less than about 8 mg/unit dose, less than about 7 mg/unit dose, less than about 6 mg/unit dose, less than about mg/unit dose, less than about 4 mg/unit dose, less than about 3 mg/unit dose, or less than about 2 mg/unit dose. In some embodiments, the chewable gel dosage form of the invention may loratadine, or a pharmaceutically acceptable salt, ester, hydrate, or solvate thereof, in an amount that is greater than about 1 mg/unit dose, greater than about 2 mg/unit dose, greater than about 3 mg/unit dose, greater than about 4 mg/unit dose, greater than about 5 mg/unit dose, greater than about 6 mg/unit dose, greater than about 7 mg/unit dose, greater than about 8 mg/unit dose, greater than about 9 mg/unit dose, greater than about 10 mg/unit dose, greater than about 15 mg/unit dose, greater than about 20 mg/unit dose, greater than about 25 mg/unit dose, greater than about 30 mg/unit dose, or greater than about 40 mg/unit dose.

In some embodiments, the chewable gel dosage form of the invention contains loratadine, or a pharmaceutically acceptable salt, ester, hydrate, or solvate thereof, in an amount having an upper limit of about 50 mg/unit dose, 40 mg/unit dose, 30 mg/unit dose, 25 mg/unit dose, 20 mg/unit dose, 15 mg/unit dose, 10 mg/unit dose, 9 mg/unit dose, 8 mg/unit dose, 7 mg/unit dose, 6 mg/unit dose, 5 mg/unit dose, 4 mg/unit dose, 3 mg/unit dose, or 2 mg/unit dose, and n independently selected lower limit of about 1 mg/unit dose, 2 mg/unit dose, 3 mg/unit dose, 4 mg/unit dose, 5 mg/unit dose, 6 mg/unit dose, 7 mg/unit dose, 8 mg/unit dose, 9 mg/unit dose, 10 mg/unit dose, 15 mg/unit dose, 20 mg/unit dose, 25 mg/unit dose, 30 mg/unit dose, or 40 mg/unit dose, where the lower limit is less than the upper limit.

In some embodiments, the chewable gel dosage form of the invention contains loratadine, or a pharmaceutically acceptable salt, ester, hydrate, or solvate thereof, in an amount of about 1 mg/unit dose, about 2 mg/unit dose, about 3 mg/unit dose, about 4 mg/unit dose, about 5 mg/unit dose, about 6 mg/unit dose, about 7 mg/unit dose, about 8 mg/unit dose, about 9 mg/unit dose, about 10 mg/unit dose, about 15 mg/unit dose, about 20 mg/unit dose, about 25 mg/unit dose, about 30 mg/unit dose, about 40 mg/unit dose, or about 50 mg/unit dose. In some embodiments, the chewable gel product contains loratadine, or a pharmaceutically acceptable salt, ester, hydrate, or solvate thereof, an amount of about 10 mg/unit dose.

In some embodiments, the amount of loratadine present in each dosage form is from 1 mg to about 100 mg. In one embodiment, the amount of loratadine present in each dosage form is from about 5 mg to about 50 mg. In another embodiment, the amount of loratadine present in each dosage form is from about 5 mg to about 10 mg per unit dose. In yet another embodiment, the amount of loratadine present in each dosage form is about 10 mg per unit dose.

The chewable gel dosage form of the invention may be administered to adults and children. For individuals of age 6 and older, loratadine may be present in an amount of about 10 mg per unit dose. For certain pediatric subjects (e.g., children between the ages of 2-6), loratadine is preferably present an amount of about 5 mg per unit dose.

In some embodiments, dosage forms containing from about 5 mg loratadine to about 10 mg loratadine have a total weight of from about 1 g to about 10 g per unit dose. Preferably, such dosage forms have a total weight of from about 1 g to about 5 g. For example, in some embodiments, the chewable gel dosage form of the invention contains about 10 mg loratadine and has a total weight of about 5 g, while in other embodiments, the chewable gel dosage form of the invention contains about 5 mg loratadine or about 10 mg loratadine and has a total weight of about 2 g.

Dosage forms containing about 10 mg loratadine per unit dose may include a loratadine concentration of from about 0.05% wt. % to about 5 wt. % based on the total weight of the dosage form. For example, such dosage forms may contain from about 0.1% wt. % to about 1 wt. % loratadine based on the total weight of the dosage form, e.g., from about 0.2% wt. % to about 0.5 wt. % based on the total mass of the dosage form.

Alternatively, loratadine may be present in the dosage form in a concentration of from about 0.01% by weight to about 2% by weight, e.g., from about 0.1% to about 1% by weight based on the total mass of the dosage form. For an adult dose, loratadine concentrations are preferably from about 0.1% by weight to about 1% by weight. For a pediatric dose (e.g., children between the ages of 2-6), loratadine concentrations are preferably from about 0.1% by weight to about 0.5% by weight based on the total mass of the dosage form.

In another embodiment of the invention, the chewable gel dosage form comprises loratadine in combination with one or more additional active pharmaceutical ingredients. The additional active pharmaceutical ingredient is selected from pseudoephedrine, chlorpheniramine, phenylephrine, guaifenesin, dextromethorphan, diphenhydramine, and combinations thereof.

Pharmaceutically acceptable salts of the additional active ingredients may also be used in combination with loratadine. In some embodiments, the additional active pharmaceutical ingredient is selected from pseudoephedrine hydrochloride, chlorpheniramine maleate, phenylephrine hydrochloride, dextromethorphan hydrobromide, diphenhydramine tartrate, and combinations thereof.

In one embodiment, chewable gel dosage form includes an active pharmaceutical ingredient comprising loratadine and pseudoephedrine. The pseudoephedrine may be present in the form of a pharmaceutically acceptable salt. Preferably, the salt form of pseudoephedrine used is the sulfate salt or hydrochloride salt. In some embodiments, the salt form of pseudoephedrine is pseudoephedrine sulfate.

In embodiments in which the chewable gel dosage form comprises loratadine and pseudoephedrine, loratadine may be present in an amount from about 2 mg to 20 mg and pseudoephedrine is present in an amount from 100 mg to 300 mg per unit dose. In some embodiments, loratadine is present in an amount from about 5 mg to about 10 mg loratadine and pseudoephedrine is present in an amount from about 120 mg to about 240 mg per unit dose.

In some embodiments, the chewable gel dosage form comprises loratadine and pseudoephedrine sulfate. In some embodiments, loratadine is present in an amount of about 10 mg and pseudoephedrine sulfate is present in an amount of about 240 mg per unit dose. For example, loratadine may be present in an amount of 10 mg and pseudoephedrine sulfate is present in an amount of 240 mg per unit dose. In another embodiments, loratadine is present in an amount of about 5 mg and pseudoephedrine sulfate is present in an amount of about 120 mg per unit dose. Preferably, loratadine is present in an amount of 5 mg and pseudoephedrine sulfate is present in an amount of 120 mg per unit dose.

In one aspect, the present invention provides a method for the temporary relief of symptoms due to hay fever and upper respiratory allergies by administering the chewable gel dosage form of the invention to an individual in need thereof. The symptoms may include runny nose, sneezing, itchy and water eyes, and itching of the nose or throat. These symptoms are also referred to as common allergy symptoms. Other methods of use of the chewable gel dosage form of the invention are directed to treatment of allergic rhinitis, uticaria and the like.

The present invention additionally provides a method for temporary relief of common allergy symptoms such as sneezing, runny nose, itchy, watery eyes and itchy nose or throat, along with nasal congestion and sinus pressure. In such embodiments, the active pharmaceutical ingredient preferably includes loratadine and pseudoephedrine.

The chewable gel dosage form of the invention may be administered at any suitable interval to provide relief for various symptoms affecting an individual. For example, the dosage form may be administered once per day or multiple times per day depending on the severity of the symptoms. Typically, the chewable gel dosage form of the invention is administered once per day.

The chewable gel dosage form of the invention preferably includes a gelling agent. The gelling agent may be present in the chewable gel dosage form in an amount from about 0.01% to about 15% by weight of the dosage form. In some embodiments, the gelling agent is present in an amount from about 0.5% to about 8% by weight of the dosage form, for example, from about 0.5% to about 1% by weight of the dosage form, from about 1% to about 1.5% by weight of the dosage form, from about 1.5% to about 2% by weight of the dosage form, from about 2% to about 2.5% by weight of the dosage form, from about 2.5% to about 3% by weight of the dosage form, from about 3% to about 3.5% by weight of the dosage form, from about 3.5% to about 4% by weight of the dosage form, from about 4% to about 4.5% by weight of the dosage form, from about 4.5% to about 5% by weight of the dosage form, from about 5% to about 5.5% by weight of the dosage form, from about 5.5% to about 6% by weight of the dosage form, from about 6% to about 6.5% by weight of the dosage form, from about 6.5% to about 7% by weight of the dosage form, or from about 7.5% to about 8% by weight of the dosage form. In some embodiments, the gelling agent is present in an amount from about 1% to about 5% by weight of the dosage form. In other embodiments, the gelling agent is present in an amount from about 5% to about 10% by weight of the dosage form. In other embodiments, the gelling agent is present in an amount from about 5% to about 15% by weight of the dosage form.

In some embodiments, the chewable gel dosage form of the invention contains one or more gelling agents in an amount of from about 0.5% w/w to about 10% w/w (wt % gelling agent(s) based on the total weight of the dosage form). For instance, the chewable gel dosage form of the invention one or more gelling agents in an amount that is less than about 10% w/w, less than about 9.5% w/w, less than about 9% w/w, less than about 8.5% w/w, less than about 8% w/w, less than about 7.5% w/w, less than about 7% w/w, less than about 6.5% w/w, less than about 6% w/w, less than about 5.5% w/w, less than about 5% w/w, less than about 4.5% w/w, less than about 4% w/w, less than about 3.75% w/w, less than about 3.5% w/w, less than about 3.25% w/w, less than about 3% w/w, less than about 2.75% w/w, less than about 2.5% w/w, less than about 2.25% w/w, less than about 2% w/w, less than about 1.75% w/w, less than about 1.5% w/w, less than about 1.25% w/w, less than about 1% w/w, or less than about 0.75% w/w.

In some embodiments, the chewable gel dosage form of the invention contains one or more gelling agents in an amount that is greater than about 0.5% w/w, greater than about 0.75% w/w, greater than about 1% w/w, greater than about 1.25% w/w, greater than about 1.5% w/w, greater than about 1.75% w/w, greater than about 2% w/w, greater than about 2.25% w/w, greater than about 2.5% w/w, greater than about 2.75% w/w, greater than about 3% w/w, greater than about 3.25% w/w, greater than about 3.5% w/w, greater than about 3.75% w/w, greater than about 4% w/w, greater than about 4.5% w/w, greater than about 5% w/w, greater than about 5.5% w/w, greater than about 6% w/w, greater than about 6.5% w/w, greater than about 7% w/w, greater than about 7.5% w/w, greater than about 8% w/w, greater than about 8.5% w/w, or greater than about 9% w/w.

In some embodiments, the chewable gel dosage form of the invention contains one or more gelling agents in an amount of at most about 10% w/w, at most about 9.5% w/w, at most about 9% w/w, at most about 8.5% w/w, at most about 8% w/w, at most about 7.5% w/w, at most about 7% w/w, at most about 6.5% w/w, at most about 6% w/w, at most about 6.5% w/w, at most about 5% w/w, at most about 4.5% w/w, at most about 4% w/w, at most about 3.75% w/w, at most about 3.5% w/w, at most about 3.25% w/w, at most about 3% w/w, at most about 2.75% w/w, at most about 2.5% w/w, at most about 2.25% w/w, at most about 2% w/w, at most about 1.75% w/w, at most about 1.5% w/w, at most 1.25% w/w, 1% w/w, or at most 0.75% w/w, and an independently selected lower limit of at least about 0.5% w/w, at least about 0.75% w/w, at least about 1% w/w, at least about 1.25% w/w, at least about 1.5% w/w, at least about 1.75% w/w, at least about 1.8% w/w, at least about 2% w/w, at least about 2.25% w/w, at least about 2.5% w/w, at least about 2.75% w/w, at least about 3% w/w, at least about 3.25% w/w, at least about 3.5% w/w, at least about 3.75% w/w, at least about 4% w/w, at least about 4.5% w/w, at least about 5.0% w/w, at least about 5.5% w/w, at least about 6.0% w/w, at least about 6.5% w/w, at least about 7.0% w/w, at least about 7.5% w/w, at least about 8.0%, at least about 8.5% w/w, or at least about 9% w/w, where the lower limit is less than the upper limit.

In some embodiments, the chewable gel dosage form of the invention contains one or more gelling agents in an amount of about 0.5% w/w, about 0.75% w/w, about 1% w/w, about 1.25% w/w, about 1.5% w/w, about 1.75% w/w, about 2% w/w, about 2.25% w/w, about 2.5% w/w, about 2.75% w/w, about 3% w/w, about 3.25% w/w, about 3.5% w/w, about 3.75% w/w, about 4% w/w, about 4.5% w/w, about 5% w/w, about 5.5% w/w, about 6.0%, about 6.5% w/w, about 7.0% w/w, about 7.5% w/w, about 8.0%, about 8.5% w/w, about 9.0%, about 9.5% w/w, or about 10% w/w. In some embodiments, the chewable gel dosage form of the invention contains one or more gelling agents in an amount of about 1% w/w. In some embodiments, the chewable gel dosage form of the invention contains the one or more gelling agents in an amount of about 2% w/w. In some embodiments, the chewable gel dosage form of the invention contains one or more gelling agents in an amount of about 5% w/w. In some embodiments, the chewable gel dosage form of the invention contains one or more gelling agents in an amount of about 6% w/w. In some embodiments, the chewable gel dosage form of the invention contains one or more gelling agents in an amount of about 7% w/w.

Any suitable gelling agent may be used to provide the dosage form with the desired characteristics including, for example, semi-solid structure, shape, texture, bioavailability, stability, and other properties as described herein. The gelling agent is preferably a USP (U.S. Pharmacopeia) grade gelling agent. In some embodiments, the gelling agent is selected from pectin, carrageenan, agar, starch, gelatin, modified starch, acacia gum, alginic acid, locust bean gum, and combinations thereof. In some embodiments, the gelling agent is pectin exclusively, carrageenan exclusively, agar exclusively, starch exclusively, gelatin exclusively, modified starch exclusively, acacia gum exclusively, alginic acid exclusively, or locust bean gum exclusively.

In some embodiments, the chewable gel dosage form of the invention includes pectin, carrageenan, agar, or a combination thereof as a gelling agent in an amount of from about 0.01% to about 15% by weight of the dosage form, e.g., from about 0.01% to about 10% by weight of the dosage form, from about 0.05% to about 10% by weight of the dosage form, from about 0.05% to about 5% by weight of the dosage form, from about 0.5% to about 10% by weight of the dosage form, from about 0.5% to about 5% by weight of the dosage form, or from about 0.75% to about 5% by weight of the dosage form. In some embodiments, pectin, carrageenan, and/or agar is present in an amount from about 1% to about 5% by weight of the dosage form, e.g., from about 1% to about 4% by weight of the dosage form, e.g., from about 1% to about 3% by weight of the dosage form. In other embodiments, pectin, carrageenan, and/or agar is present in an amount from about 2% to about 3% by weight of the dosage form. In other embodiments, the chewable gel dosage form of the invention includes gelatin, starch, or a combination thereof as a gelling agent in an amount of from about 1% to about 15% by weight of the dosage form, e.g., from about 5% to about 15% by weight of the dosage form, or from about 5% to about 10% by weight of the dosage form.

Any suitable pectin may be used as a gelling agent in the chewable gel dosage form of the invention. Pectin is a purified polysaccharide typically obtained by aqueous extraction from fruit peal, specifically citrus peel or apple pomace. Commercial pectin is typically produced by extraction of edible plant material in hot, acidified water followed by isolation of the pectin from the ensuing solution. The dominant raw material is the rind of citrus fruit. Apple pomace and sugar beet pulp are also used as sources of pectin. Pectin is principally a heterogeneous polymer in which the dominant component is a linear chain of 1→4 linked galacturonic acid in which a proportion of the carboxyl acid groups are present as methyl esters and, in amidated pectin, are replaced with amide groups. The linear backbone of a typical pectin polymer is a homogalacturonan built by sequences of α(1→4) linked D-galacturonic acid residues. The various sub-structural entities in pectin polymers may vary with the extraction methodology and raw material used in the manufacture thereof. The free acid groups of polygalacturonic acid building blocks can be esterified as the methyl esters and/or may be partly or fully neutralized with cations such as sodium, potassium, calcium, or ammonium. The ratio of esterified galacturonic acid groups to total galacturonic acid groups is often referred to as the degree of esterification (DE). Generally, the highest DE that normally achieved by extraction of natural raw material is approximately 80%. Pectin with DE from 5%-75% is produced by controlled de-esterification in the manufacturing process. A DE of 50% conventionally divides commercial pectin products into HM (high methyl ester or high methoxy) and LM (low methyl ester or low methoxy) pectin. The DE can have a significant influence on the properties of pectin. Pectin molecules generally contain regions of neutral sugars, such as arabinose, galactose, and rhamnose. The chemical composition of pectin can vary at all levels, e.g., there may be regions of different composition within one molecule, differences in average composition between molecules, and differences in average composition between sample lots. In typical commercial products, the molecular weight distribution is fairly broad with an average of about 100-200 kDa. Pectins suitable for use in the chewable gel dosage form of the invention include, for example, high-methoxy pectin and low-methoxy pectin and combinations thereof. Low-methoxy pectin, which is amidated, is often referred to as LMA pectin, and also may be used as a gelling agent. Examples of suitable pectins that may be used as a gelling agent in the chewable gel dosage form of the invention include, for example, Genu® citrus pectin USP/100 (from CP Kelco), Genu® citrus pectin USP/200 (CP Kelco), Genu® pectin DC-200-B (CP Kelco), Genu® pectin DC slow set-Z (CP Kelco), Pectin Classic CS 509 (Herbstreith & Fox), Pectin Classic CS 501 (Herbstreith & Fox), pectin GP 1105 (Milazzo), pectin CN2776 (Milazzo), and Cargill Unipectine™ 759 CS MB HM pectin. In some embodiments, the gelling agent is pectin in a concentration of from about 1% to about 5% by weight of the dosage form. For example, in some embodiments, the gelling agent is a Genu® citrus pectin USP/100 or a Genu® citrus pectin USP/200 (available through CP Kelco) in a concentration of from about 1% to about 5% by weight of the dosage form.

Any suitable carrageenan may be used as a gelling agent in the chewable gel dosage form of the invention. Carrageenan is generally obtained by extraction with water or alkaline water of certain red seaweed species of the class Rhodophyceae. Carrageenan is a hydrocolloid that consists primarily of potassium, sodium, magnesium, and/or calcium sulfate esters of galactose and 3,6-anhydrogalactose copolymers. The relative proportion of cations existing in carrageenan may be modified during processing to the extent that one may become predominant. Lambda carrageenan is generally defined as being void of 3,6-anhydro-D-galactose units, highly sulfated, and readily soluble under most conditions. Kappa carrageenan is generally defined as containing 3,6-anhydro-D-galactose as part of the repeating unit and fewer sulfate groups. Kappa carrageenan is generally less hydrophilic and less soluble than lambda carrageenan. Iota carrageenan, is generally defined as more hydrophilic than kappa carrageenan by virtue of its 2-sufate, which, in addition to its position, counteracts the less hydrophilic character of the 3,6-anhydro-D-galactose residue. Examples of suitable carrageenans that may be used as a gelling agent in the chewable gel dosage form of the invention include, e.g., Carrageenan Genutine® Type 310-C, and Gelcarin GP 611 ((FMC) Danisco-DuPont). In some embodiments, the gelling agent is carrageenan in a concentration of from about 1% to about 5% by weight of the dosage form. For example, in some embodiments, the gelling agent is Carrageenan Genutine® Type 310-C in a concentration of from about 1% to about 5% by weight of the dosage form.

Any suitable agar may be used as a gelling agent in the chewable gel dosage form of the invention. Examples of suitable agars that may be used as a gelling agent in the chewable gel dosage form of the invention is Agar Ticagel® Nat GC-581 B (supplied as a blend containing agar, sucrose, and locust bean gum), Agar RS-111 (TIC GUMS, a purified agar), and Ticagel® GC 564 S (TIC GUMS, supplied as blend of agar, modified corn starch, pectin and cellulose gum). In some embodiments, the gelling agent is agar in a concentration of from about 1% to about 5% by weight of the dosage form. For example, in some embodiments, the gelling agent is Agar Ticagel® Nat GC-581 B in a concentration of from about 1% to about 5% by weight of the dosage form.

Any suitable starch may be used as a gelling agent in the chewable gel dosage form of the invention. Suitable starches that may be used as gelling agent in the chewable gel dosage form of the invention include high amylose modified food starches. In some embodiments, the gelling agent is a high amylose modified food starch, which is present in a concentration of from about 5% to about 15% by weight of the dosage form. In some embodiments, the chewable gel dosage form of the invention includes a starch gelling agent (e.g., a high amylose modified food starch) in an amount of from about 0.01% to about 20% by weight of the dosage form. For example, a starch gelling agent (e.g., a high amylose modified food starch) may be present in in an amount from about 1% to about 15% by weight of the dosage form, e.g., from about 5% to about 15% by weight of the dosage form, from about 5% to about 10% by weight of the dosage form, from about 10% to about 15% by weight of the dosage form, or from about 8% to about 12% by weight of the dosage form. Suitable starches include, e.g., CornStarch Hi-Set 322 (Ingredion, a modified corn starch), and CargillSet 05130 (CARGILL, also a modified corn starch).

Any suitable gelatin may be used as a gelling agent in the chewable gel dosage form of the invention. Gelatin generally consists of a mixture of fractions composed almost entirely of amino acids joined by peptide linkages to form polymers varying in molecular mass from about 15,000 to about 400,000 kDa. Gelatin derived from an acid-treated precursor is generally known as Type A and gelatin derived from an alkali-treated process is generally known as Type B. The gelatin may be animal-derived gelatin, chemically-modified gelatin, physically-modified gelatin, or a combination thereof. Preferred gelatins include 200-275 bloom gelatins, e.g., 250-275 bloom gelatins. The gelling agent used in the chewable gel dosage form of the invention may include one or more gelatins derived from any suitable source such as, for example, porcine (e.g., pigskin (e.g., Gelatin 275 Pig Skin), or bovine (e.g., bovine bone). Suitable gelatin gelling agents currently may be obtained from several suppliers, which include, e.g., Gelita, Nitta Gelatin, Weishardt International, and PB Leiner. Alternatively, the gelatin may be a hydrolyzed gelatin, also commonly known as hydrolyzed collagen, collagen hydrolysate, and collagen peptide. Hydrolyzed gelatin having a molecular weight ranging from about 2,500 to about 5,000 may be used. An example of a suitable hydrolyzed gelatin is Peptiplus® powder from Gelita. In some embodiments, the gelling agent is Gelatin 275 Bloom Pig Skin, which is present in a concentration of from about 1% to about 10% by weight of the dosage form.

For example, the chewable gel dosage form of the invention may include a gelatin gelling agent (e.g., Gelatin 275 Pig Skin, bovine bone, and/or hydrolyzed gelatin) in an amount from about 0.01% to about 15% by weight of the dosage form. For example, the chewable gel dosage form of the invention may include gelatin (e.g., Gelatin 275 Pig Skin, bovine bone, and/or hydrolyzed gelatin) in an amount of from about 0.5% to about 10% by weight by weight of the dosage form, for example from about 0.5% to about 1% by weight of the dosage form, from about 1% to about 1.5% by weight of the dosage form, from about 1.5% to about 2% by weight of the dosage form, from about 2% to about 2.5% by weight of the dosage form, from about 2.5% to about 3% by weight of the dosage form, from about 3% to about 3.5% by weight of the dosage form, from about 3.5% to about 4% by weight of the dosage form, from about 4% to about 4.5% by weight of the dosage form, from about 4.5% to about 5% by weight of the dosage form, from about 5% to about 5.5% by weight of the dosage form, from about 5.5% to about 6% by weight of the dosage form, from about 6% to about 6.5% by weight of the dosage form, from about 6.5% to about 7% by weight of the dosage form, from about 7% to about 7.5% by weight of the dosage form, or from about 7.5% to about 8% by weight of the dosage form. In some embodiments, gelatin (e.g., Gelatin 275 Pig Skin, bovine bone, and/or hydrolyzed gelatin) is present in an amount from about 1% to about 5% by weight of the dosage form. In other embodiments, gelatin (e.g., Gelatin 275 Pig Skin, bovine bone, and/or hydrolyzed gelatin) is present in an amount from about 5% to about 10% by weight of the dosage form, e.g., from about 7% to about 8% by weight of the dosage form.

The chewable gel dosage form of the invention may further include one or more additional components such as, for example, polyols, sugar, corn syrup, propylene glycol, glycerin, pH adjusting agents, flavorants, and colorants, or a combination thereof.

For example, the chewable gel dosage form of the invention may include one or more polyols. Suitable polyols may include, for example, isomalt, lactitol, maltitol, mannitol, sorbitol, erythritol, polyglycitol, xylitol, polyol mixtures that exist in certain hydrogenated starch hydrolysates (HSHs), and combinations thereof. For example, the chewable gel dosage form of the invention may include maltitol, sorbitol, xylitol, or a combination thereof, e.g., maltitol and sorbitol, maltitol and xylitol, sorbitol and xylitol, or maltitol, xylitol and sorbitol. Polyols may be supplied in any suitable form including, e.g., as a syrup or powder. For example, xylitol and sorbitol are often supplied in powder form, whereas maltitol may be supplied as a powder or as a syrup, e.g., in which maltitol is the dominant polyol. Maltitol is a disaccharide that may be produced by hydrogenation of maltose obtained from starch. Maltitol syrup may be obtained as a hydrogenated starch hydrolysate (HSH), e.g., from the hydrogenation of corn syrup which is a mixture of carbohydrates produced from the partial hydrolysis of starch. It will be appreciated that certain grades of HSH may contain sorbitol, maltitol, one or more longer chain polyols, e.g., maltotriitol, other sugar-related carbohydrates, and mixtures thereof depending, in part, upon the degree of hydrolysis of the starch hydrolysate precursor. Generally, when no single polyol is dominant in the HSH, the mixture may be referred to generically as hydrogenated starch hydrosylate (HSH), whereas if, e.g., about 50% or more of the polyols in the mixture are of one type, the HSH may be labeled as a syrup of the dominant polyol, e.g., “sorbitol syrup,” “maltitol syrup,” etc. Some grades of maltitol syrup may contain about 50% to about 80% maltitol by weight with the remainder being, e.g., mostly sorbitol in combination with a lower quantity of other sugar-related substances. One (non-limiting) example of a suitable maltitol syrup is Lycasin® 85/55 HSH (Maltitol) Syrup, supplied by Roquette.

The chewable gel dosage form of the invention may include one or more polyols in an amount of, e.g., from about 20% to about 80% by weight of the dosage form, e.g., from about 30% to about 80% by weight of the dosage form. In some embodiments, one or more polyols may be present in an amount from about 40% to about 80% by weight of the dosage form, for example, about 40% to about 50% by weight of the dosage form, about 50% to about 60% by weight of the dosage form, about 60% to about 70% by weight of the dosage form, about 50% to about 80% by weight of the dosage form, about 60% to about 80% by weight of the dosage form, about 70% to about 80% by weight of the dosage form, about 45% to about 55% by weight of the dosage form, and about 55% to about 65% by weight of the dosage form. Alternatively, one or more polyols may be present in an amount from about 65% to about 75% by weight of the dosage form. In other embodiments, one or more polyols may be present in an amount from about 65% to about 80% by weight of the dosage form, or in an amount from about 75% to about 80% by weight of the dosage form.

In some embodiments, the chewable gel dosage form of the invention includes maltitol syrup in an amount of from about 40% to about 80% by weight of the dosage form. In such embodiments, the chewable gel dosage form of the invention may include maltitol syrup in an amount of, e.g., from about 40% to about 50% by weight of the dosage form, from about 50% to about 60% by weight of the dosage form, or from about 60% to about 70% by weight of the dosage form.

In some embodiments, the chewable gel dosage form of the invention includes sorbitol (e.g., sorbitol powder) in an amount of from about 1% to about 25% by weight of the dosage form. In such embodiments, the chewable gel dosage form of the invention may include sorbitol (e.g., sorbitol powder) in an amount of, e.g., from about 1% to about 15% by weight of the dosage form, from about 5% to about 15% by weight of the dosage form, or from about 10% to about 20% by weight of the dosage form.

In some embodiments, the chewable gel dosage form of the invention includes xylitol (e.g., xylitol powder) in an amount of from about 1% to about 40% by weight of the dosage form. In such embodiments, the chewable gel dosage form of the invention may include xylitol (e.g., xylitol powder) in an amount of, e.g., from about 10% to about 30% by weight of the dosage form.

In some embodiments, the chewable gel dosage form of the invention comprises maltitol syrup in an amount from about 40% to about 50% by weight of the dosage form, and sorbitol (e.g., sorbitol powder) in an amount from about 5% to about 15% by weight of the dosage form.

In some embodiments, the chewable gel dosage form of the invention comprises maltitol syrup in an amount from about 60% to about 70% by weight of the dosage form, and sorbitol (e.g., sorbitol powder) in an amount from about 10% to about 20% by weight of the dosage form.

In some embodiments, the chewable gel dosage form of the invention comprises maltitol syrup in an amount from about 40% to about 80% by weight of the dosage form, xylitol (e.g., xylitol powder) in an amount from about 1% to about 40% by weight of the dosage form, and sorbitol (e.g., sorbitol powder) in an amount from about 1% to about 15% by weight of the dosage form.

In other embodiments, the chewable gel dosage form of the invention comprises maltitol syrup in an amount from about 30% to about 60% by weight of the dosage form, xylitol (e.g., xylitol powder) in an amount from about 5% to about 40% by weight of the dosage form, and sorbitol (e.g., sorbitol powder) in an amount from about 1% to about 15% by weight of the dosage form.

In other embodiments, the chewable gel dosage form of the invention comprises maltitol syrup in an amount from about 40% to about 50% by weight of the dosage form, xylitol (e.g., xylitol powder) in an amount from about 10% to about 30% by weight of the dosage form, and sorbitol (e.g., sorbitol powder) in an amount from about 5% to about 15% by weight of the dosage form.

In some embodiments, the chewable gel dosage form of the invention comprises maltitol syrup, xylitol powder, and sorbitol powder, wherein the ratio of maltitol syrup to xylitol powder is from about 0.5:1 to about 3.5:1, the ratio of maltitol syrup to sorbitol powder is from about 3:1 to about 6:1, and the ratio of xylitol powder to sorbitol powder is from about 1:1 to about 4:1. For example, in such dosage forms, the ratio of maltitol syrup to xylitol powder may be from about 1.5:1 to about 2.5:1, the ratio of maltitol syrup to sorbitol powder may be from about 4:1 to about 5:1, and the ratio of xylitol powder to sorbitol powder may be from about 2:1 to about 3:1.

In some embodiments, the chewable gel dosage form of the invention comprises a mixture of maltitol syrup, and sorbitol powder, wherein the ratio of maltitol syrup to xylitol powder is from about 3:1 to about 6:1. For example, in such dosage forms, the ratio of maltitol syrup to sorbitol powder may be from about 4:1 to about 5:1.

Any suitable ratio of polyol to gelling agent can be used in the chewable gel dosage form of the invention. Suitable ratios of polyol to gelling agent may include, e.g., ratios of from about 40:1 to about 1:1 (polyol to gelling agent) by dry weight. Suitable ratios of polyol to gelling agent also may include ratios of from about 30:1 to about 10:1 by dry weight. Suitable ratios of polyol to gelling agent further may include ratios of from about 35:1 to about 25:1 by dry weight, or from about 35:1 to about 30:1 by dry weight.

In some embodiments, the chewable gel dosage form of the invention contains a polyol and a pectin gelling agent (e.g., Genu® citrus pectin USP/100 or a Genu® citrus pectin USP/200 (available through CP Kelco)). In such embodiments, suitable ratios of polyol to pectin can be in the range of from about 40:1 to about 1:1 polyol to pectin by dry weight, or from about 30:1 to about 10:1 polyol to pectin by dry weight. Suitable ratios of polyol to pectin also can range from about 35:1 to about 25:1 polyol to pectin by dry weight, or from about 35:1 to about 30:1 polyol to pectin by dry weight. In such embodiments, the polyol can include, for example maltitol in an amount from about 40% by weight to about 50% by weight, xylitol in an amount from about 20% by weight to about 30% by weight, and sorbitol in an amount from about 5% by weight to about 15% by weight. In some embodiments, the gelling agent includes pectin and the polyol includes maltitol syrup alone or in combination with one or more additional polyols as described herein. For example, the chewable gel dosage form of the invention may include pectin as the gelling agent in an amount of from about 1% to about 5% by weight of the dosage form, and, e.g., maltitol syrup in an amount of from about 40% to about 80% by weight of the dosage form.

In some embodiments, the chewable gel dosage form of the invention comprises pectin (e.g., Genu® citrus pectin USP/100 or a Genu® citrus pectin USP/200 (available through CP Kelco)) as a gelling agent, and a mixture of maltitol syrup, xylitol powder and sorbitol powder, wherein the ratio of maltitol syrup to xylitol powder is from about 0.5:1 to about 3.5:1, the ratio of maltitol syrup to sorbitol powder is from about 3:1 to about 6:1, and/or the ratio of xylitol powder to sorbitol powder is from about 1:1 to about 4:1. In such embodiments, the ratio of maltitol syrup to xylitol powder also can range, e.g., from about 1.5:1 to about 2.5:1, the ratio of maltitol syrup to sorbitol powder can range, e.g., from about 4:1 to about 5:1, and the ratio of xylitol powder to sorbitol powder can range, e.g., from about 2:1 to about 3:1.

Alternatively, in certain embodiments, the chewable gel dosage form of the invention comprises pectin (e.g., Genu® citrus pectin USP/100 or a Genu® citrus pectin USP/200 (available through CP Kelco)) as a gelling agent, and a mixture of maltitol syrup and sorbitol powder, wherein the ratio of maltitol syrup to xylitol powder is from about 3:1 to about 6:1. In such embodiments, the ratio of maltitol syrup to sorbitol powder also can range from about 4:1 to about 5:1. In such embodiments, the polyol can include, for example maltitol in an amount from about 40% by weight to about 50% by weight, xylitol in an amount from about 20% by weight to about 30% by weight, and sorbitol in an amount from about 5% by weight to about 15% by weight.

In other embodiments, the chewable gel dosage form of the invention contains a polyol and a gelatin gelling agent (e.g., Gelatin 275 Bloom Pig Skin). For example, in such embodiments, the chewable gel dosage form of the invention may include, e.g., maltitol syrup in an amount of from about 40% to about 80% by weight of the dosage form, and gelatin (e.g., Gelatin 275 Bloom Pig Skin) as the gelling agent in an amount of from about 1% to about 10% by weight of the dosage form.

The chewable gel dosage form of the invention also may include a surfactant, which preferably comprises a polyoxyethylene sorbitan ester, e.g., a polysorbate. Suitable polysorbates include, for example, polysorbate 80 (polyoxyethylene (20) sorbitan monooleate), also commonly known as Tween 80. The surfactant may be present in any suitable concentration, e.g., in a concentration of from about 0.005% to about 2% by weight of the dosage form, from about 0.01% to about 2% by weight of the dosage form, from about 0.01% to about 1% by weight of the dosage form, from about 0.01% to about 0.75% by weight of the dosage form, from about 0.01% to about 0.5% by weight of the dosage form, or from about 0.01% to about 0.1% by weight of the dosage form, e.g., from about 0.01% to about 0.05% by weight of the dosage form. In some embodiments, the chewable gel dosage form contains polysorbate 80 in an amount of from about 0.01% to about 0.5% by weight of the dosage form, e.g., polysorbate 80 in an amount of from about 0.01% to about 0.05% by weight of the dosage form.

The chewable gel dosage form of the invention also may include a suspending agent, e.g., to stabilize a suspension used in formulating the chewable gel dosage form, e.g., by lowering the sedimentation rate of particles in suspension. The suspending agent may include a hydrophilic colloid, e.g., which spontaneously forms a colloidal dispersion with water. Suitable suspending agents may include, e.g., synthetic and semi-synthetic compounds, polysaccharides, starches, inorganic salts, and combinations thereof. In some embodiments, polyvinylpyrrolidone or povidone may be used as a suspending agent in the chewable gel dosage form of the invention. Suitable suspending agents include povidone products sold under the trademark Kollidon®.

The chewable gel dosage form of the invention also may include a sugar in any suitable amount. For example, the chewable gel dosage form of the invention also may include a sugar in an amount from about 5% to about 80% by weight of the chewable gel dosage form, e.g., from about 5% to about 50% by weight of the dosage form. In some embodiments, sugar may be present in an amount of from about 10% to about 80% by weight of the dosage form, for example, from about 20% to about 80% by weight of the dosage form, from about 10% to about 70% by weight of the dosage form, from about 10% to about 60% by weight of the dosage form, from about 10% to about 50% by weight of the dosage form, from about 10% to about 40% by weight of the dosage form, from about 10% to about 30% by weight of the dosage form, from about 10% to about 20% by weight of the dosage form, from about 20% to about 70% by weight of the dosage form, from about 20% to about 65% by weight of the dosage form, from about 20% to about 60% by weight of the dosage form, from about 25% to about 60% by weight of the dosage form, from about 30% to about 60% by weight of the dosage form, from about 35% to about 60% by weight of the dosage form, from about 40% to about 60% by weight of the dosage form, from about 45% to about 60% by weight of the dosage form, from about 50% to about 60% by weight of the dosage form, from about 55% to about 60% by weight of the dosage form, from about 20% to about 30% by weight of the dosage form, or from about 30% to about 40% by weight of the dosage form.

In some embodiments, the chewable gel dosage form of the invention comprises one or more polyols and one or more sugars, wherein the ratio of polyol to sugar may be from about 1:10 to about 10:1 by dry weight. Exemplary ratios of polyol to sugar can include ratios of from about 1:2 to about 2:1 polyol to sugar by dry weight, for example, from about 1:1.5 to about 1:5.1 polyol to sugar by dry weight.

In other embodiments, the chewable gel dosage form of the invention is substantially free of sugar or sugar free. In some embodiments, the substantially sugar free or sugar free chewable gel dosage form of the invention contains one or more polyols as described herein.

In some embodiments, the chewable gel dosage form of the invention is free of artificial sweeteners. In other embodiments, the chewable gel dosage form of the invention contains only non-caloric or artificial sweeteners.

In some embodiments, the chewable gel dosage form of the invention is vegetarian or vegan. In some embodiments, the chewable gel dosage form of the invention is Kosher or Parve.

In other embodiments, the chewable gel dosage form of the invention includes corn syrup. Corn syrup may be present without a polyol. Alternatively, corn syrup may be present in combination with a polyol. Any suitable corn syrup may be used, for example, corn syrup having 36-65 DE (dextrose equivalents), e.g., corn syrup 42 DE, corn syrup 43 DE, or corn syrup 63 DE. Corn syrup may contain about 50% by weight to about 90% by weight solids, preferably about 80% solids. Corn syrup may be present in the chewable gel dosage form in any suitable amount, for example, in an amount of from about 20% to about 80% by weight of the dosage form. In some embodiments, corn syrup may be present in an amount from about 20% to about 70% by weight of the dosage form, from about 20% to about 60% by weight of the dosage form, from about 20% to about 50% by weight of the dosage form, from about 20% to about 40% by weight of the dosage form, or from about 20% to about 30% by weight of the dosage form. In other embodiments, corn syrup may be present in an amount from about 30% to about 80% by weight of the dosage form, for example, from about 40% to about 80% by weight of the dosage form, from about 50% to about 80% by weight of the dosage form, from about 30% to about 70% by weight of the dosage form, from about 40% to about 70% by weight of the dosage form, from about 50% to about 70% by weight of the dosage form, from about 30% to about 60% by weight of the dosage form, from about 30% to about 50% by weight of the dosage form, or from about 30% to about 40% by weight of the dosage form. In some embodiments, the ratio of corn syrup to sugar is from about 1:10 to about 10:1 by dry weight. For example, the ratio of corn syrup to sugar may be from about 1:2 to about 2:1 by dry weight, e.g., from about 1:1.5 to about 1:5.1. In other embodiments, the ratio of corn syrup to gelling agent is from about 20:1 to about 1:1 by dry weight. For example, the ratio of corn syrup to gelling agent may be from about 10:1 to about 2:1 by dry weight, e.g., from about 10:1 to about 3:1 by dry weight.

The chewable gel dosage form of the invention also may include glucose syrup in any suitable amount, for example, in an amount of from about 30% to about 70% by weight of the dosage form, e.g., from about 30% to about 70% by weight of the dosage form, from about 35% to about 65% by weight of the dosage form, from about 40% to about 60% by weight of the dosage form, or from about 50% to about 60% by weight of the dosage form. Any suitable glucose syrup may be used in the chewable gel dosage form of the invention. Suitable glucose syrups include, e.g., high maltose glucose syrups, e.g., Glucose Syrup 54DE High-Maltose.

The chewable gel dosage form of the invention also may include a hydrogenated starch hydrolysate (HSH) as described herein. It will be appreciated that certain grades of HSH may contain sorbitol, maltitol, one or more longer chain polyols, e.g., maltotriitol, other sugar-related carbohydrates, and mixtures thereof depending upon the degree of hydrolysis of the starch hydrolysate precursor. Exemplary HSHs may contain, for example, substantial quantities of hydrogenated oligo- and poly-saccharides in addition to monomeric and dimeric polyols. Examples of commercially available HSH include Hystar® 3375 syrup (75% solids), Hystar® 4075 and Hystar® 6075 supplied by Ingredion Inc. Other commercially available HSH include 75/400 from Roquette and Stabilite® liquid HSH, Roquette Lycasin® 80/55 HSH (Maltitol) Syrup (a hydrogenated, partially hydrolyzed, starch which includes a mixture of polyols containing mainly D-maltitol (at least 50%) in combination with D-sorbitol and various hydrogenated oligo- and polysaccharides), and Stabilite® powdered HSH supplied by Ingredion Inc.

The chewable gel dosage form of the invention also may include glycerin, also commonly known as glycerol, which may function as an emollient. Preferably, glycerin USP is used. In some embodiments, glycerin is present in the chewable gel dosage form and gelatin is absent. Glycerin may be present in the chewable gel dosage form of the invention in an amount from about 0.1% to about 10% by weight of the dosage form. For example, glycerin may be present in an amount from about 0.5% to about 5% by weight of the dosage form, for example from about 0.5% to about 1% by weight of the dosage form, from about 1% to about 1.5% by weight of the dosage form, from about 1.5% to about 2.0% by weight of the dosage form, from about 2.0% to about 2.5% by weight of the dosage form, from about 2.5% to about 3.0% by weight of the dosage form, from about 3.0% to about 3.5% by weight of the dosage form, from about 3.5% to about 4.0% by weight of the dosage form, from about 4.0% to about 4.5% by weight of the dosage form, and from about 4.5% to about 5.0% by weight of the dosage form. In some embodiments, the chewable gel dosage form of the invention contains about 2% glycerin by weight of the dosage form.

The chewable gel dosage form may optionally include a pH adjusting agent or buffer. Any suitable pH adjusting agent or buffer may be used that is sufficient to adjust the pH during the manufacture of the dosage form to yield the desired pH or pH range. Two or more pH adjusting agents may be used. By way of example, the pH adjusting agent or buffer may include any suitable acid, salt thereof, or derivative thereof (e.g., acid anhydride derivatives, etc.). Suitable pH adjusting agents or buffers may include, for example, citric acid, fumaric acid, malic acid, phosphoric acid, succinic acid, tartaric acid, maleic acid, acetic acid, phosphoric acid, hydrochloric acid, lactic acid, propionic acid, salts thereof, and derivatives thereof (e.g., acid anhydride derivatives, etc.), or any suitable combination of two or more of the foregoing. In some embodiments, the pH adjusting agent or buffer may include sodium citrate, citric acid, sodium ascorbate, ascorbic acid, or a combination of two or more of the foregoing. The compounds used in the pH adjusting agent or buffer may be supplied in solid form (e.g., as a powder) or in aqueous solution. For example, citric acid may be supplied in a 50% solution. In some embodiments, the pH adjusting agent or buffer is sodium citrate, citric acid, or a combination thereof. Preferably, both sodium citrate and citric acid are included in the chewable gel dosage form as a pH adjusting agent or buffer.

The pH adjusting agent or buffer may be present in the chewable gel dosage form in an amount from about 0.1% to about 5% by weight of the dosage form. In some embodiments, the pH adjusting agent or buffer is present in an amount from about 1% to about 5% by weight of the dosage form, for example, from about 1% to about 1.5% by weight of the dosage form, from about 1.5% to about 2% by weight of the dosage form, from about 2% to about 2.5% by weight of the dosage form, from about 2.5% to about 3% by weight of the dosage form, from about 3% to about 3.5% by weight of the dosage form, from about 3.5% to about 4.0% by weight of the dosage form, from about 4% to about 4.5% by weight of the dosage form, or from about 4.5% to about 5% by weight of the dosage form.

In some embodiments, sodium citrate is present in an amount from about 0.1% to about 1% by weight of the dosage form. For example, sodium citrate may be present in an amount from about 0.1% to about 0.5% by weight by weight of the dosage form, for example, from about 0.1% to about 0.2% by weight of the dosage form, from about 0.2% to about 0.3% by weight of the dosage form, from about 0.3% to about 0.4% by weight of the dosage form, or from about 0.4% to about 0.5% by weight of the dosage form.

In other embodiments, citric acid is present (as 50% aqueous solution) in an amount from about 0.5% to about 3% by weight of the dosage form, for example from about 0.5% to about 1% by weight of the dosage form, from about 1% to about 1.5% by weight of the dosage form, from about 1.5% to about 2% by weight of the dosage form, from about 2% to about 2.5% by weight of the dosage form, and from about 2.5% to about 3% by weight of the dosage form.

For example, the chewable gel dosage form of the invention may include sodium citrate and citric acid, wherein the sodium citrate concentration is from about 0.1% to about 1% by weight of the dosage form, e.g., from about 0.1% to about 0.5% by weight of the dosage form, for example, from about 0.1% to about 0.2%, from about 0.2% to about 0.3% by weight of the dosage form, from about 0.3% to about 0.4% by weight of the dosage form, or from about 0.4% to about 0.5% by weight of the dosage form, and the citric acid (e.g., as a 50% solution) concentration is from about 0.5% to about 3% by weight of the dosage form, for example from about 0.5% to about 1% by weight of the dosage form, from about 1% to about 1.5% by weight of the dosage form, from about 1.5% to about 2% by weight of the dosage form, from about 2% to about 2.5% by weight of the dosage form, or from about 2.5% to about 3% by weight of the dosage form.

The chewable gel dosage form also may include one or more flavoring agents. Any suitable food-grade flavorant or flavorant composition may be used, e.g., to suppress the bitterness of one or more active ingredients and/or to provide a pleasant taste to the dosage form upon chewing and swallowing. A mixture of two or more flavorants also may be used to yield the desired taste characteristic. Suitable flavorants include natural and/or artificial sweeteners such as, for example, sucralose, acesulfame potassium, stevia, sodium saccharine, erythritol, and aspartame. Another suitable flavorant may be a fraction of the lactone group such as, for example, decalactone and dodecalactone (e.g., gamma dodecalactone). Lactone fractions are typically supplied in a propylene glycol solution, in particular from 0.5% to 1% in propylene glycol solution. The flavorant may be orange or cherry flavors. Alternatively, the flavorant may be menthol. In some embodiments, the chewable gel dosage form of the invention includes Cherry Flavor FFS (223G12) in an amount of from about 0.01% to about 2% by weight of the dosage form, e.g., about 0.3% Cherry Flavor FFS (223G12) by weight of the dosage form. The flavorant (or flavorant composition) may be present in any suitable amount, e.g., in an amount up to about 1% by weight of the dosage form, e.g., up to about 0.5% by weight of the dosage form, up to about 0.01% of the dosage form, up to about 0.05% of the dosage form, up to about 0.1% of the dosage form, up to about 0.2% of the dosage form, up to about 0.3% of the dosage form, up to about 0.4% of the dosage form, or up to about 0.5% of the dosage form. If the flavorant includes a lactone group, fractions of the lactone group may be present in an amount of, e.g., from about 1 ppm to 50 ppm, preferably from about 2 ppm to about 10 ppm, and more preferably from about 3 ppm to about 9 ppm.

The chewable gel dosage form also may include one or more colorants, e.g., to provide a suitable appearance for the chewable gel dosage form. Examples of suitable colorants include red or yellow dyes such as FD&C Red #40, FD&C Yellow #5, FD&C Yellow #6, D&C Reds 3, 22, 28, 33 and 36, D&C Yellow 10, FD&C Blues 1 and 2, FD&C Green 3, red iron oxide, caramel, beta-carotene, carmine, and combinations thereof.

The chewable gel dosage form of the invention or the formulation used in its manufacture (e.g., the final blend or composition of components to be deposited into suitable molds before curing of final dosage forms) may further comprise water or residual moisture. In some embodiments, the chewable gel dosage form of the invention generally has a water content, or a residual moisture content, of less than about 20% by weight of the dosage form, e.g., up to about 19% by weight of the dosage form, up to about 18% by weight of the dosage form, up to about 17% by weight, up to about 16% by weight of the dosage form, up to about 15% by weight of the dosage form, e.g., about 14% by weight of the dosage form or less, about 13% by weight of the dosage form or less, about 12% by weight of the dosage form or less, about 11% by weight of the dosage form or less, about 10% by weight of the dosage form or less, about 9% by weight of the dosage form or less, about 8% by weight of the dosage form or less, about 7% by weight of the dosage form or less, about 6% by weight of the dosage form or less, or about 5% by weight of the dosage form or less. In some embodiments, the water content of the chewable gel dosage form of the invention is from about 5% to about 20% by weight of the dosage form. In some embodiments, the water content of the chewable gel dosage form of the invention is from about 8% to about 15% by weight of the dosage form, e.g., from about 9% to about 15% by weight of the dosage form, from about 8% to about 12% by weight of the dosage form, or from about 14% to about 15% by weight of the dosage form. In some embodiments, the formulation used in the manufacture of the chewable gel dosage form of the invention, e.g., the final blend deposited into pre-formed molds to produce individual unit dosage forms of the invention, has a water content of from about 10% to about 25% by weight of the dosage form, e.g., from about 12% to about 22% water by weight of the dosage form, from about 13% to about 22% water by weight of the dosage form, from about 14% to about 22% water by weight of the dosage form, or from about 12% to about 22% water by weight of the dosage form.

The water content of the chewable gel dosage form of the invention may be determined by any suitable method such as, for example, by Karl Fischer analysis. Water content may be determined by Karl Fischer analysis employing techniques that are general known to those of skill in the art, and may include the use of commercially available equipment such as, for example, a suitable Karl Fischer titrator supplied by Mettler-Toledo, LLC, Columbus, Ohio (United States).

In some embodiments, the formulation used in the manufacture of the chewable gel dosage form of the invention comprises loratadine in an amount sufficient to provide 10 mg of loratadine per unit dose (e.g., 10 mg of loratadine per 5 g of final unit dosage form), from about 1 to about 5% of a suitable pectin gelling agent, from about 40% to about 80% maltitol syrup, from about 1% to about 40% xylitol powder, from about 1% to about 15% sorbitol powder, from about 0.1% to about 1% sodium citrate, from about 0.5% to about 3% citric acid (50/50 solution) (dry basis), from about 13% to about 22% water, from about 0.1% to about 10% glycerin (e.g., glycerin USP), from about 0.01% to about 0.5% of a polysorbate surfactant (e.g., Tween 80), of a suitable a coloring agent (e.g., FD&C Red #40), and from about 0.01% to about 2% of a suitable flavoring agent (e.g., Cherry Flavor FFS (223G12)), by weight of the formulation.

In other embodiments, the formulation used in the manufacture of the chewable gel dosage form of the invention comprises loratadine in an amount sufficient to provide 10 mg of loratadine per unit dose (e.g., 10 mg of loratadine per 5 g of final unit dosage form), from about 1% to about 10% of a suitable gelatin gelling agent (e.g., Gelatin 275 Bloom Pig Skin), from about 40% to about 80% maltitol syrup, from about 0.5% to about 3% citric acid (50/50 solution) (dry basis), from about 14% to about 22% water, from about 0.1% to about 10% glycerin (e.g., glycerin USP), from about 0.01% to about 0.5% of a polysorbate surfactant (e.g., Tween 80), of a suitable a coloring agent (e.g., FD&C Red #40), and from about 0.01% to about 2% of a suitable flavoring agent (e.g., Cherry Flavor FFS (223G12)), by weight of the formulation.

In other embodiments, the formulation used in the manufacture of the chewable gel dosage form of the invention comprises loratadine in an amount sufficient to provide 10 mg of loratadine per unit dose (e.g., 10 mg of loratadine per 5 g of final unit dosage form), from about 20% to about 60% sugar, from about 20% to about 60% of a suitable corn syrup (e.g., Corn Syrup 63DE(, from about 5% to about 15% of a suitable starch gelling agent (e.g., modified food starch (high amylose)), from about 0.1% to about 1% sodium citrate, from about 0.5% to about 3% citric acid (50/50 solution) (dry basis), from about 15% to about 22% water, from about 0.1% to about 10% glycerin (e.g., glycerin USP), from about 0.01% to about 0.5% of a polysorbate surfactant (e.g., Tween 80), of a suitable a coloring agent (e.g., FD&C Red #40), and from about 0.01% to about 2% of a suitable flavoring agent (e.g., Cherry Flavor FFS (223G12)), by weight of the formulation.

In other embodiments, the formulation used in the manufacture of the chewable gel dosage form of the invention comprises loratadine in an amount sufficient to provide 10 mg of loratadine per unit dose (e.g., 10 mg of loratadine per 5 g of final unit dosage form), from about 1% to about 5% of a suitable agar gelling agent (e.g., Agar Ticagel® Nat GC-581 B), from about 10% to about 60% sugar, from about 20% to about 70% of a suitable corn syrup (e.g., Corn Syrup 43DE), from about 0.5% to about 3% citric acid (50/50 solution) (dry basis), from about 14% to about 22% water, from about 0.1% to about 10% glycerin (e.g., glycerin USP), from about 0.01% to about 0.5% of a polysorbate surfactant (e.g., Tween 80), of a suitable a coloring agent (e.g., FD&C Red #40), and from about 0.01% to about 2% of a suitable flavoring agent (e.g., Cherry Flavor FFS (223G12)), by weight of the formulation.

In other embodiments, the formulation used in the manufacture of the chewable gel dosage form of the invention comprises loratadine in an amount sufficient to provide 10 mg of loratadine per unit dose (e.g., 10 mg of loratadine per 5 g of final unit dosage form), from about 30% to about 70% of a suitable glucose syrup (e.g., Glucose Syrup 54DE High-Maltose), from about 1% to about 5% of a suitable carrageenan gelling agent (e.g., Carrageenan Genutine® Type 310-C), from about 0.1% to about 1% sodium citrate, from about 5% to about 50% sugar, from about 0.5% to about 3% citric acid (50/50 solution) (dry basis), from about 13% to about 22% water, from about 0.1% to about 10% glycerin (e.g., glycerin USP), from about 0.01% to about 0.5% of a polysorbate surfactant (e.g., Tween 80), of a suitable a coloring agent (e.g., FD&C Red #40), and from about 0.01% to about 2% of a suitable flavoring agent (e.g., Cherry Flavor FFS (223G12)), by weight of the formulation.

In some embodiments, the chewable gel dosage form of the invention comprises pectin as a gelling agent, maltitol in an amount from about 60% to about 70% by weight of the dosage form, and sorbitol in an amount from about 10% to about 20% by weight of the dosage form. In other embodiments, the chewable gel dosage form of the invention comprises pectin as a gelling agent, and a sugar alcohol, which includes maltitol syrup in an amount from about 40% to about 50% by weight of the dosage form, xylitol powder in an amount from about 10% to about 30% by weight of the dosage form, and sorbitol powder in an amount from about 5% to about 15% by weight of the dosage form. In the embodiments described in this paragraph, the chewable gel dosage form may further include, e.g., a pH adjusting agent or buffer (e.g., sodium citrate, citric acid, or a combination thereof), an emollient (e.g., glycerin), a surfactant (e.g., polysorbate 80), a coloring agent, a flavoring agent, or a combination thereof, as described herein, in the amounts described herein, a loratadine concentration of from about 0.1% to about 0.3%, e.g., about 0.2%, by weight of the dosage form, and a pectin concentration of from about 1% to about 5%, e.g., from about 2% to about 3%, by weight of the dosage form.

In other embodiments, the chewable gel dosage form of the invention comprises pectin as a gelling agent, maltitol syrup in an amount from about 50% to about 60% by weight of the dosage form, xylitol powder in an amount from about 10% to about 20% by weight of the dosage form, and sorbitol powder in an amount from about 5% to about 10% by weight of the dosage form. In other embodiments, the chewable gel dosage form of the invention comprises pectin as a gelling agent, and a sugar alcohol, which includes maltitol syrup in an amount from about 60% to about 70% by weight of the dosage form, and sorbitol powder in an amount from about 10% to about 15% by weight of the dosage form. In the embodiments described in this paragraph, the chewable gel dosage form may further include, e.g., a pH adjusting agent or buffer (e.g., sodium citrate, citric acid, or a combination thereof), an emollient (e.g., glycerin), a surfactant (e.g., polysorbate 80), a coloring agent, a flavoring agent, or a combination thereof, as described herein, in the amounts described herein, a loratadine concentration of from about 0.1% to about 0.3%, e.g., about 0.2%, by weight of the dosage form, and a pectin concentration of from about 1% to about 5%, e.g., from about 2% to about 3%, by weight of the dosage form.

In some embodiments, the chewable gel dosage form of the invention comprises gelatin as a gelling agent, maltitol syrup in an amount from about 40% to about 80%, e.g., from about 60% to about 70%, by weight of the dosage form. In such embodiments, the chewable gel dosage form may further include, e.g., a pH adjusting agent or buffer (e.g., sodium citrate, citric acid, or a combination thereof), an emollient (e.g., glycerin), a surfactant (e.g., polysorbate 80), a coloring agent, a flavoring agent, or a combination thereof, as described herein, in the amounts described herein, a loratadine concentration of from about 0.1% to about 0.3%, e.g., about 0.2%, by weight of the dosage form, and a gelatin concentration of from about 1% to about 10%, e.g., from about 5% to about 10%, by weight of the dosage form.

In some embodiments, the chewable gel dosage form of the invention comprises a starch gelling agent (e.g., a high amylose starch), sugar in an amount of from about 20% to about 60% by weight of the dosage form, and corn syrup in an amount of from about 20% to about 60% by weight of the dosage form. Such embodiments include chewable gel dosage forms that contain a starch gelling agent (e.g., a high amylose starch) and, e.g., sugar in an amount of from about 30% to about 40% by weight of the dosage form, and corn syrup in an amount of from about 25% to about 35% by weight of the dosage form. In the embodiments described in this paragraph, the chewable gel dosage form may further include, e.g., a pH adjusting agent or buffer (e.g., sodium citrate, citric acid, or a combination thereof), an emollient (e.g., glycerin), a surfactant (e.g., polysorbate 80), a coloring agent, a flavoring agent, or a combination thereof, as described herein, in the amounts described herein, a loratadine concentration of from about 0.1% to about 0.3%, e.g., about 0.2%, by weight of the dosage form, and a starch gelling agent concentration of from about in an amount of from about 5% to about 15%, e.g., from about 9% to about 11%, by weight of the dosage form, and may be substantially free of polyols as described herein.

In other embodiments, the chewable gel dosage form of the invention comprises agar as a gelling agent, sugar in an amount of from about 10% to about 60% by weight of the dosage form, and corn syrup in an amount of from about 20% to about 70% by weight of the dosage form. Such embodiments include chewable gel dosage forms that contain Agar as a gelling agent and, e.g., sugar in an amount of from about 20% to about 30% by weight of the dosage form, and corn syrup in an amount of from about 40% to about 50% by weight of the dosage form. In the embodiments described in this paragraph, the chewable gel dosage form may further include, e.g., a pH adjusting agent or buffer (e.g., sodium citrate, citric acid, or a combination thereof), an emollient (e.g., glycerin), a surfactant (e.g., polysorbate 80), a coloring agent, a flavoring agent, or a combination thereof, as described herein, in the amounts described herein, a loratadine concentration of from about 0.1% to about 0.3%, e.g., about 0.2%, by weight of the dosage form, and an Agar gelling agent concentration of from about in an amount of from about 1% to about 5%, e.g., from about 2% to about 4%, by weight of the dosage form, and may be substantially free of polyols as described herein.

In other embodiments, the chewable gel dosage form of the invention comprises carrageenan as a gelling agent, and glucose syrup (e.g., high maltose glucose syrup) in an amount of from about 30% to about 70% by weight of the dosage form, and sugar in an amount of from about 5% to about 50% by weight of the dosage form. Such embodiments include chewable gel dosage forms that contain carrageenan as a gelling agent and, e.g., glucose syrup (e.g., high maltose glucose syrup) in an amount of from about 50% to about 60% by weight of the dosage form, and sugar in an amount of from about 10% to about 20% by weight of the dosage form. In the embodiments described in this paragraph, the chewable gel dosage form may further include, e.g., a pH adjusting agent or buffer (e.g., sodium citrate, citric acid, or a combination thereof), an emollient (e.g., glycerin), a surfactant (e.g., polysorbate 80), a coloring agent, a flavoring agent, or a combination thereof, as described herein, in the amounts described herein, a loratadine concentration of from about 0.1% to about 0.3%, e.g., about 0.2%, by weight of the dosage form, and an carrageenan gelling agent concentration of from about in an amount of from about 1% to about 5%, e.g., from about 2% to about 4%, or from about 2% to about 3%, by weight of the dosage form, and may be substantially free of polyols as described herein.

In other embodiments, the formulation used in the manufacture of the chewable gel dosage form of the invention comprises a therapeutically effective amount of one or more active agents (or a pharmaceutically acceptable salt, ester, hydrate or solvate thereof), e.g., loratadine in an amount of about 10 mg; pectin in an amount from about 1% to about 4% by weight of the dosage form; sugar in an amount from about 40% to about 80% by weight of the dosage form; hydrolyzed starch hydrolysate in an amount from about 40% to about 80% by weight of the dosage form; hydrolyzed gelatin in an amount from about 0.5% to about 8% by weight of the dosage form; sodium citrate in an amount from about 0.1% to about 1% by weight of the dosage form; and citric acid in an amount from about 0.5% to about 3% by weight of the dosage form, wherein the water content is from about 12% to about 22% by weight of the dosage form.

In other embodiments, the formulation used in the manufacture of the chewable gel dosage form of the invention comprises a therapeutically effective amount of one or more active agents (or a pharmaceutically acceptable salt, ester, hydrate or solvate thereof), e.g., loratadine in an amount of about 10 mg; pectin in an amount from about 1% to about 4% by weight of the dosage form; sugar in an amount from about 40% to about 80% by weight of the dosage form; corn syrup in an amount from about 40% to about 80% by weight of the dosage form; hydrolyzed gelatin in an amount from about 0.5% to about 8% by weight of the dosage form; sodium citrate in an amount from about 0.1% to about 1% by weight of the dosage form; and citric acid in an amount from about 0.5% to about 3% by weight of the dosage form, wherein the water content is from about 12% to about 22% by weight of the dosage form.

In other embodiments, the formulation used in the manufacture of the chewable gel dosage form of the invention comprises a therapeutically effective amount of one or more active agents (or a pharmaceutically acceptable salt, ester, hydrate or solvate thereof), e.g., loratadine in an amount of about 10 mg; pectin in an amount from about 1% to about 4% by weight of the dosage form; sugar in an amount from about 40% to about 80% by weight of the dosage form; hydrolyzed starch hydrolysate in an amount from about 40% to about 80% by weight of the dosage form; glycerin in an amount from about 0.1% to about 5% by weight of the dosage form; sodium citrate in an amount from about 0.1% to about 1% by weight of the dosage form; and citric acid in an amount from about 0.5% to about 3% by weight of the dosage form, wherein the water content is from about 12% to about 22% by weight of the dosage form.

The chewable gel dosage form of the invention may be prepared by any suitable method including, for example, a batch process or a continuous process. In some embodiments, the components of the dosage form are combined together in a suitable vessel. The components may be combined in any suitable order.

The chewable gel dosage form of the invention may be manufactured, e.g., by adding a gelling agent (e.g. pectin, gelatin, carrageenan, agar, modified food starch, etc.) to water and/or a sweetener premix with one or more pH adjusting agent, if desired, and mixing sufficiently (e.g., for a sufficient time and/or to a desired temperature) to achieve desired properties, e.g., consistency and/or homogeneity. The resulting mixture may be combined with additional components, for example, one or more bulk sweeteners (e.g. sugar, corn syrups, high-maltose corn syrup, etc.), one or more polyols (e.g., maltitol syrup, maltitol, sorbitol, xylitol, erythritol, mannitol, isomalt, etc.), and one or more other components described herein, to form a base composition for the chewable gel dosage form. The water content of the base composition can range, e.g., from about 10% to about 90% by weight of the base composition. In some embodiments, the base composition has a water content of from about 10% to about 50% by weight of the base composition, for example, a water content of from about 15% to about 25% by weight of the base composition, about 25% to about 30% by weight of the base composition, about 30% to about 35% by weight of the base composition, about 35% to about 40% by weight of the base composition, about 40% to about 45% by weight of the base composition, or about 45% to about 50% by weight of the base composition.

In some embodiments, the base composition may be heated (or “cooked”), e.g., under pressure, at a suitable temperature, e.g., to remove at least a portion of the water. By reducing the water content, e.g., by heating (or cooking, or by any other suitable means), the base may be converted into a chewable gel dosage form having desired physical characteristics, e.g., viscosity, consistency, texture, etc. The base may be cooked by any suitable means including, for example, with a steam-jacketed vessel or a conventional heat exchanger. Cooking or heating may optionally be carried out with the aid of a vacuum or under reduced pressure.

The base composition may be cooked at any suitable temperature and for a sufficient length of time to yield a molten mass having the desired water content. In some embodiments, the base composition is cooked sufficient to yield a residual moisture content of from about 5% to about 25% by weight of the base composition. For example, the base composition may be heated/cooked sufficient to provide a residual moisture content of from about 9% to about 20% by weight of the base composition, for example, a residual moisture content of from about 9% to about 10% by weight of the base composition, about 10% to about 11% by weight of the base composition, about 11% to about 12% by weight of the base composition, about 12% to about 13% by weight of the base composition, about 13% to about 14% by weight of the base composition, about 14% to about 15% by weight of the base composition, about 15% to about 16% by weight of the base composition, about 16% to about 17% by weight of the base composition, about 17% to about 18% by weight of the base composition, about 18% to about 19% by weight of the base composition, or about 19% to about 20% by weight of the base composition. In certain embodiments, the residual moisture content of the base composition after cooking is reduced sufficiently such that the final semi-solid dosage form contains from about 0.01% to about 2% by weight of the active ingredient(s) used therein.

Any suitable temperature can be used for cooking the base composition. Suitable temperatures for cooking the based composition may from about 210° F. to about 330° F. For example, in some embodiments, the base composition may be cooked at a temperature of from about 220° F. to about 260° F., e.g., from about 220° F. to about 230° F., about 230° F. to about 240° F., from about 240° F. to about 250° F., or from about 250° F. to about 260° F.

After the base composition is cooked sufficiently to yield the desired properties, e.g., to a workable molten mass, any remaining components to be included in the semi-solid, chewable gel dosage form may be added, for example, a therapeutically effective amount of one or more active agents (or a pharmaceutically acceptable salt, ester, hydrate or solvate thereof), e.g., loratadine or a pharmaceutically acceptable salt thereof, water, a hydrocolloid, thickening agent, or gelling agent (e.g., a hydrolyzed gelatin), an emollient (e.g., glycerin), a surfactant (e.g., Tween 80), a suspending agent, a flavorant, a colorant, or a combination of any of the foregoing, to form a final blend. These additional components may be added to the base by any suitable means, for example, mass flow meters, static mixers, and the like, or by any suitable combination of such means.

A pH adjusting agent such as, e.g., citric acid, sodium citrate, or one or more other pH adjusting agents as described herein, or a combination thereof, also may be added to the base composition, e.g., to provide a suitable pH for the final blend that contains all of the components of the semi-solid dosage form. In some embodiments, the pH of the chewable gel dosage form of the invention or the pH of the final blend, for example, before gelation, e.g., while the product is still in a molten or liquid state, may generally range from about 3 to about 10, e.g., from about 3 to about 8, from about 3.5 to about 8.5, from about 3.5 to about 4.5, from about 4.5 to about 5.5, from about 5.5 to about 6.5, from about 6.5 to about 7.5, or from about 7.5 to about 8.5.

In some embodiments, the pH of the chewable gel dosage form of the invention or the pH of the final blend is from about 3 to about 8, from about 3 to about 7.5, from about 3 to about 7, from about 3 to about 6.5, from about 3 to about 6, from about 3 to about 5.5, from about 3 to about 5, from about 3 to about 4.5, from about 3 to about 4, from about 3 to about 3.5, from about 3.5 to about 8, from about 3.5 to about 7.5, from about 3.5 to about 7, from about 3.5 to about 6.5, from about 3.5 to about 6, from about 3.5 to about 5.5, from about 3.5 to about 5, from about 3.9 to about 5, from about 3.5 to about 4.5, from about 3.5 to about 4, from about 4 to about 8, from about 4 to about 7.5, from about 4 to about 7, from about 4 to about 6.5, from about 4 to about 6, from about 4 to about 5.5, from about 4 to about 5, from about 4 to about 4.5, from about 4.5 to about 8, from about 4.5 to about 7.5, from about 4.5 to about 7, from about 4.5 to about 6.5, from about 4.5 to about 6, from about 4.5 to about 5.5, from about 4.5 to about 5, from about 5 to about 8, from about 5 to about 7.5, from about 5 to about 7, from about 5 to about 6.5, from about 5 to about 6, from about 5 to about 5.5, from about 5.5 to about 8, from about 5.5 to about 7.5, from about 5.5 to about 7, from about 5.5 to about 6.5, from about 5.5 to about 6, from about 6 to about 8, from about 6 to about 7.5, from about 6 to about 7, from about 6 to about 6.5, from about 6.5 to about 8, from about 6.5 to about 7.5, from about 6.5 to about 7, from about 7 to about 8, from about 7 to about 7.5, or from about 7.5 to about 7.5. In some embodiments, the pH of the final blend is about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 6.5, about 7.6, about 7.7, about 7.8, about 6.9, or about 8.

In some embodiments, the pH of the chewable gel dosage form of the invention or the pH of the final blend is from about 2.4 to about 5.5, from about 2.4 to about 5, from about 2.4 to about 4.5, from about 2.4 to about 4, from about 2.4 to about 3.5, from about 2.4 to about 3.4, from about 2.4 to about 3.3, from about 2.4 to about 3.2, from about 2.4 to about 3.1, from about 2.4 to about 3, from about 2.4 to about 2.9, from about 2.4 to about 2.8, from about 2.4 to about 2.7, from about 2.4 to about 2.6, from about 2.4 to about 2.5, from about 2.5 to about 5.5, from about 2.5 to about 5, from about 2.5 to about 4.5, from about 2.5 to about 4, from about 2.5 to about 3.5, from about 2.5 to about 3.4, from about 2.5 to about 3.3, from about 2.5 to about 3.2, from about 2.5 to about 3.1, from about 2.5 to about 3, from about 2.5 to about 0.9, from about 2.5 to about 2.6, from about 2.6 to about 5.5, from about 2.6 to about 5, from about 2.6 to about 4.5, from about 2.6 to about 4, from about 2.6 to about 3.5, from about 2.6 to about 3.4, from about 2.6 to about 3.3, from about 2.6 to about 3.2, from about 2.6 to about 3.1, from about 2.6 to about 3, from about 2.6 to about 2.9, from about 2.6 to about 2.7, from about 2.7 to about 5.5, from about 2.7 to about 5, from about 2.7 to about 4.5, from about 2.7 to about 4, from about 2.7 to about 3.5, from about 2.7 to about 3.4, from about 2.7 to about 3.3, from about 2.7 to about 3.2, from about 2.7 to about 3.1, from about 2.7 to about 3, from about 2.7 to about 2.9, from about 2.7 to about 2.8, from about 2.8 to about 5.5, from about 2.8 to about 5, from about 2.8 to about 4.5, from about 2.8 to about 4, from about 2.8 to about 3.5, from about 2.8 to about 3.4, from about 2.8 to about 3.3, from about 2.8 to about 3.2, from about 2.8 to about 3.1, from about 2.8 to about 3, from about 2.8 to about 2.9, from about 2.9 to about 5.5, from about 2.9 to about 5, from about 2.9 to about 4.5, from about 2.9 to about 4, from about 2.9 to about 3.5, from about 2.9 to about 3.4, from about 2.9 to about 3.3, from about 2.9 to about 3.2, from about 2.9 to about 3.1, from about 2.9 to about 3, from about 3 to about 5.5, from about 3 to about 5, from about 3 to about 4.5, from about 3 to about 4, from about 3 to about 3.5, from about 3 to about 3.4, from about 3 to about 3.3, from about 3 to about 3.2, from about 3 to about 3.1, from about 3.1 to about 5.5, from about 3.1 to about 5, from about 3.1 to about 4.5, from about 3.1 to about 4, from about 3.1 to about 3.5, from about 3.1 to about 3.4, from about 3.1 to about 3.3, from about 3.1 to about 3.2, from about 3.2 to about 5.5, from about 3.2 to about 5, from about 3.2 to about 4.5, from about 3.2 to about 4, from about 3.2 to about 3.5, from about 3.2 to about 3.4, from about 3.2 to about 3.3, from about 3.3 to about 5.5, from about 3.3 to about 5, from about 3.3 to about 4.5, from about 3.3 to about 4, from about 3.3 to about 3.5, from about 3.3 to about 3.4, from about 3.4 to about 5.5, from about 3.4 to about 5, from about 3.4 to about 4.5, from about 3.4 to about 4, from about 3.4 to about 3.5, from about 3.5 to about 5.5, from about 3.5 to about 5, from about 3.5 to about 4.5, from about 3.5 to about 4, from about 4 to about 5.5, from about 4 to about 5, from about 4 to about 4.5, from about 4.5 to about 5.5, from about 4.5 to about 5, or from about 5 to about 5.5. In some embodiments, the pH of the chewable gel dosage form of the invention or the pH of the final blend, is about 2.4, about 2.5, about 2.6, about 2.7, about 2.8, about 2.9, about 3.0, about 3.1, about 3.2, about 3.3, about 3.4, about 3.5, about 4, about 4.5, about 5, or about 5.5.

In some embodiments, different blends of components are prepared separately and then combined together to form a final blend from which the semi-solid dosage form is obtained. For example, a primary blend may be combined with a secondary blend to form the final blend. A separate blend containing, e.g., one or more flavorants, one or more colorants, an acid solution (e.g., containing citric acid), and/or any other agent or reagent that may induce gelation may optionally be added for preparation of the final blend.

The final blend may be further processed as needed prior to preparation of the semi-solid dosage form. For example, the final blend may be transferred to a depositor hopper having a jacket to maintain a temperature of, e.g., from about 140° F. to about 210° F., or from about 150° F. to about 200° F. In some embodiments, the final blend may be transferred to a depositor hopper having a jacket to maintain a temperature of, e.g., from about 160° F. to about 170° F., from about 170° F. to about 180° F., from about 180° F. to about 190° F., or from about 190° F. to about 200° F. After the desired physical and/or chemical characteristics are achieved, the final blend may be dispensed from the depositor hopper, e.g., into pre-formed molds, to produce the chewable gel dosage form of the invention.

Advantageously, a pre-determined amount of the final blend, for example, based on weight or volume, e.g., to achieve a desired dosage of active agent depending on the concentration thereof in the final blend (accounting for any additional water loss that may occur), may be dispensed to form individual pieces. The individual pieces preferably contain the desired amount of the active ingredients as described herein. For example, individual pieces may each contain 10 mg loratadine or a pharmaceutically acceptable salt thereof, 5 mg loratadine or a pharmaceutically acceptable salt thereof, or 10 mg loratadine or a pharmaceutically acceptable salt thereof and 240 mg pseudoephedrine sulfate.

In accordance with the invention, a chewable gel dosage of the invention, which contains about 10 mg loratadine and at least one gelling agent, may administered orally to produce achieve therapeutically relevant pharmacokinetic responses in subjects. The primary pharmacokinetic parameters include the Area Under the Curve for concentration at time t (AUC(0-t)), Area Under the Curve for concentration extrapolated to infinity (AUC(0-inf)), and the maximum plasma concentration (Cₘₐₓ). The pharmacokinetic parameters are based on concentrations of loratadine and its primary metabolite descarboethoxyloratadine, commonly known as desloratadine.

The method and chewable gel dosage form may be characterized by AUC(0-t) and AUC(0-inf) of loratadine alone or in combination with Cₘₐₓ of loratadine. Alternatively, the method and chewable gel dosage form may be characterized by Cₘₐₓ of loratadine alone.

Alternatively, the method and chewable gel dosage form may be characterized by AUC(0-t) and AUC(0-inf) of desloratadine alone or in combination with Cₘₐₓ of desloratadine. Alternatively, the method and chewable gel dosage form may be characterized by Cₘₐₓ of desloratadine alone.

In another alternative, the method and chewable gel dosage form may be characterized by AUC(0-t) and AUC(0-inf) of loratadine in combination with the AUC(0-t) and AUC(0-inf) of desloratadine. Also, the method and chewable gel dosage form may be characterized by Cₘₐₓ of loratadine and Cₘₐₓ of desloratadine. In a further alternative, the method and chewable dosage form may be characterized by AUC(0-t) and AUC(0-inf) of loratadine, AUC(0-t) and AUC(0-inf) of desloratadine, Cₘₐₓ of loratadine and Cₘₐₓ of desloratadine.

The pharmacokinetic parameters for loratadine and desloratadine obtained from the method of the invention may be for an individual subject or a number of subjects having a suitable population. In one embodiment, the pharmacokinetic parameters are obtained following administration of a single chewable gel dosage form of the invention containing about 10 mg of loratadine to a subject population of at least 16 subjects.

Where the chewable gel dosage form of the invention is administered to a population of subjects, the resulting pharmacokinetic parameters may be expressed as arithmetic means using untransformed data or geometric means based on log transformed data. Preferably, the mean pharmacokinetic parameters for a subject population are geometric means. In addition, the pharmacokinetic parameters may include data obtained from all subjects in the subject population or only those subjects for which complete data has been obtained (i.e., blood samples have been obtained at all relevant time points).

In embodiments of the invention where the chewable gel dosage form is administered under fasted condition, a subject has not ingested food for at least 8 hours prior to dosing. Preferably, under fasted conditions, a subject has not ingested food for at least 10 hours prior to dosing. For fasted conditions, the dosage form may be taken at any time of the day, preferably after an overnight fast of at least 10 hours prior to dosing.

When the chewable dosage form is administered under fed condition, a subject has eaten a high-fat, high-calorie meal an hour or less prior to dosing. Preferably, under fed conditions, a subject has eaten a high-fat, high-calorie meal 30 minutes or less prior to dosing. For fed conditions, the dosage form may be taken at any time of day, preferably after a suitable breakfast. The breakfast is typically preceded by a fast of at least 8 hours, and preferably at least 10 hours.

In accordance with an embodiment, following administration of a chewable gel dosage form of the invention comprising about 10 mg loratadine under fasted or fed conditions, AUC(0-t) and AUC(0-inf) of loratadine are generally from about 3 hr*ng/mL to about 25 hr*ng/mL. In some embodiments, AUC(0-t) and AUC(0-inf) of loratadine are from about 4 hr*ng/mL to about 22 hr*ng/mL. Alternatively, AUC(0-t) and AUC(0-inf) of loratadine are from about 7 hr*ng/mL to about 20 hr*ng/mL. Under fasted or fed conditions, Cₘₐₓ of loratadine is generally from about 2 ng/mL to about 10 ng/mL.

In accordance with another embodiment, following administration of a chewable gel dosage form of the invention comprising about 10 mg loratadine under fasted or fed conditions, AUC(0-t) and AUC(0-inf) of desloratadine are generally from about 25 hr*ng/mL to about 80 hr*ng/mL. Under fasted or fed conditions, Cₘₐₓ of desloratadine is generally from about 1 ng/mL to about 8 ng/mL. Alternatively, under fasted or fed conditions, Cₘₐₓ of desloratadine is from about 2 ng/mL to about 5 ng/mL.

In one embodiment, following administration of a chewable gel dosage form of the invention comprising about 10 mg loratadine in a fasted state, AUC(0-t) and AUC(0-inf) of loratadine are typically from about 5 hr*ng/mL to about 25 hr*ng/mL. More specifically, following administration of such a dosage form in a fasted state, AUC(0-t) and AUC(0-inf) of loratadine are from about 6 hr*ng/mL to about 15 hr*ng/mL or, alternatively, from about 12 hr*ng/mL to about 25 hr*ng/mL.

In another embodiment, the Cₘₐₓ of loratadine following administration of a chewable gel dosage form of the invention comprising about 10 mg loratadine under fasted conditions is typically from about 2 ng/mL to about 10 ng/mL. More specifically, following administration of such a dosage form in a fasted state, Cₘₐₓ of loratadine is from about 2 ng/mL to about 4 ng/mL or, alternatively, from about 5 ng/mL to about 9 ng/mL.

Alternatively, following administration of a chewable gel dosage form of the invention comprising about 10 mg loratadine in a fasted state, AUC(0-t) and AUC(0-inf) of desloratadine are typically from about 25 hr*ng/mL to about 80 hr*ng/mL. More specifically, following administration of such a dosage form in a fasted state, AUC(0-t) and AUC(0-inf) of desloratadine are from about 40 hr*ng/mL to about 75 hr*ng/mL or, alternatively, from about 30 hr*ng/mL to about 60 hr*ng/mL.

The Cₘₐₓ of desloratadine following administration of a chewable gel dosage form of the invention comprising about 10 mg loratadine under fasted conditions is typically from about 1 ng/mL to about 8 ng/mL. Alternatively, under fasted conditions, Cₘₐₓ of desloratadine is from about 2 ng/mL to about 5 ng/mL.

In another embodiment, following administration of a chewable gel dosage form of the invention comprising about 10 mg loratadine in a fed state, AUC(0-t) and AUC(0-inf) of loratadine are typically from about 10 hr*ng/mL to about 30 hr*ng/mL. More specifically, following administration of such a dosage form in a fasted state, AUC(0-t) and AUC(0-inf) of loratadine are from about 10 hr*ng/mL to about 20 hr*ng/mL.

In another embodiment, the Cₘₐₓ of loratadine following administration of a chewable gel dosage form of the invention comprising about 10 mg loratadine under fed conditions is typically from about 2 ng/mL to about 7 ng/mL. More specifically, following administration of such a dosage form in a fasted state, Cₘₐₓ of loratadine is from about 3 ng/mL to about 6 ng/mL.

Alternatively, following this administration of a chewable gel dosage form of the invention comprising about 10 mg loratadine in a fed state, AUC(0-t) and AUC(0-inf) of desloratadine is from about 35 hr*ng/mL to about 75 hr*ng/mL. More specifically, AUC(0-t) and AUC(0-inf) of desloratadine is from about 40 hr*ng/ml to about 70 hr*ng/ml.

The Cₘₐₓ of desloratadine following administration of a chewable gel dosage form of the invention comprising about 10 mg loratadine under fed conditions is typically from about 1 ng/mL to about 8.5 ng/ml. Alternatively, following administration of a chewable gel dosage form of the invention comprising about 10 mg loratadine under fed conditions, Cₘₐₓ of desloratadine is from about 2 ng/mL to about 4.5 ng/mL.

Based on the pharmacokinetic characteristics observed from administration of a chewable gel dosage form of the invention containing about 10 mg loratadine under the conditions set forth below in Example 6 (Tables 6A-6H), Example 7 (Tables 7A-7E) and Example 8 (Tables 8A-8F), acceptable bioequivalent ranges are determined to provide an equivalent treatment level. Based on current FDA criteria, a product has a bioequivalent effect if it provides AUC(0-t), AUC(0-inf) and Cₘₐₓ levels that are about 80% to about 125% of the reference product (in this case, the test products in Examples 6-8).

The ranges of bioequivalent pharmacokinetic parameters following administration of a chewable gel dosage form of the invention comprising about 10 mg loratadine under fed conditions in Example 6 are set forth in Table A below.

TABLE A

| | AUC(0-t) | AUC(0-inf) | Cₘₐₓ |
|---|---|---|---|
| | hr*ng/ml | hr*ng/ml | ng/ml |
| Loratadine | 11.001 to 17.189 | 11.764 to 18.781 | 3.339 to 5.218 |
| Desloratadine | 42.577 to 66.526 | 46.560 to 72.766 | 2.597 to 4.058 |

In one embodiment of the invention, the administration of a chewable gel dosage form of the invention comprising about 10 mg loratadine under fed conditions produces one or more of the following pharmacokinetic parameters:

AUC(0-t) of loratadine from about 11 hr*ng/ml to about 18 hr*ng/ml and AUC(0-inf) of loratadine from about 11 hr*ng/ml to about 19 hr*ng/ml;

Cₘₐₓ of loratadine from about 3 ng/ml to about 6 ng/ml;

AUC(0-t) of desloratadine from about 42 hr*ng/ml to about 67 hr*ng/ml and AUC(0-inf) of desloratadine from about 46 hr*ng/ml to about 73 hr*ng/ml; and

Cₘₐₓ of desloratadine from about 2 ng/ml to about 5 mg/ml.

In a further embodiment of the invention, the administration of a chewable gel dosage form comprising about 10 mg loratadine under fed conditions produces one or more of the following pharmacokinetic parameters:

AUC(0-t) of loratadine from about 11.0 hr*ng/ml to about 17.2 hr*ng/ml and AUC(0-inf) of loratadine from about 11.7 hr*ng/ml to about 18.8 hr*ng/ml;

Cₘₐₓ of loratadine from about 3.3 ng/ml to about 5.2 ng/ml;

AUC(0-t) of desloratadine from about 42.5 hr*ng/ml to about 66.6 hr*ng/ml and AUC(0-inf) of desloratadine from about 46.5 hr*ng/ml to about 72.8 hr*ng/ml; and

Cₘₐₓ of desloratadine from about 2.5 ng/ml to about 4.1 mg/ml.

In another embodiment of the invention, the administration of a chewable gel dosage form comprising about 10 mg loratadine under fed conditions produces one or more of the following pharmacokinetic parameters:

AUC(0-t) of loratadine from about 11.001 hr*ng/ml to about 17.189 hr*ng/ml and AUC(0-inf) of loratadine from about 11.764 hr*ng/ml to about 18.781 hr*ng/ml;

Cₘₐₓ of loratadine from about 3.339 ng/ml to about 5.218 ng/ml;

AUC(0-t) of desloratadine from about 42.577 hr*ng/ml to about 66.526 hr*ng/ml and AUC(0-inf) of desloratadine from about 46.570 hr*ng/ml to about 72.766 hr*ng/ml; and

Cₘₐₓ of desloratadine from about 2.597 ng/ml to about 4.058 mg/ml.

The ranges of bioequivalent pharmacokinetic parameters following administration under fasted conditions in Example 7 are set forth in Table B below.

TABLE B

| | AUC(0-t) | AUC(0-inf) | Cₘₐₓ |
|---|---|---|---|
| | hr*ng/ml | hr*ng/ml | ng/ml |
| Loratadine | 7.343 to 11.474 | 7.555 to 11.805 | 2.493 to 3.895 |
| Desloratadine | 42.786 to 66.853 | 46.954 to 73.365 | 2.371 to 3.705 |

In one embodiment of the invention, the administration of a chewable gel dosage form of the invention comprising about 10 mg loratadine under fasted conditions produces one or more of the following pharmacokinetic parameters:

AUC(0-t) of loratadine from about 7 hr*ng/ml to about 12 hr*ng/ml and AUC(0-inf) of loratadine from about 7 hr*ng/ml to about 12 hr*ng/ml;

Cₘₐₓ of loratadine from about 2 ng/ml to about 4 ng/ml;

AUC(0-t) of desloratadine from about 42 hr*ng/ml to about 67 hr*ng/ml and AUC(0-inf) of desloratadine from about 46 hr*ng/ml to about 74 hr*ng/ml; and

Cₘₐₓ of desloratadine from about 2 ng/ml to about 4 mg/ml.

In a further embodiment of the invention, the administration of a chewable gel dosage form of the invention comprising about 10 mg loratadine under fasted conditions produces one or more of the following pharmacokinetic parameters:

AUC(0-t) of loratadine from about 7.3 hr*ng/ml to about 11.5 hr*ng/ml and AUC(0-inf) of loratadine from about 7.5 hr*ng/ml to about 11.9 hr*ng/ml;

Cₘₐₓ of loratadine from about 2.4 ng/ml to about 3.9 ng/ml;

AUC(0-t) of desloratadine from about 42.7 hr*ng/ml to about 66.9 hr*ng/ml and AUC(0-inf) of desloratadine from about 46.9 hr*ng/ml to about 73.4 hr*ng/ml; and

Cₘₐₓ of desloratadine from about 2.3 ng/ml to about 3.805 mg/ml.

In another embodiment of the invention, the administration of a chewable gel dosage form comprising about 10 mg loratadine under fasted conditions produces one or more of the following pharmacokinetic parameters:

AUC(0-t) of loratadine from about 7.343 hr*ng/ml to about 11.474 hr*ng/ml and AUC(0-inf) of loratadine from about 7.555 hr*ng/ml to about 11.805 hr*ng/ml;

Cₘₐₓ of loratadine from about 2.493 ng/ml to about 3.895 ng/ml;

AUC(0-t) of desloratadine from about 42.786 hr*ng/ml to about 66.853 hr*ng/ml and AUC(0-inf) of desloratadine from about 46.954 hr*ng/ml to about 73.365 hr*ng/ml; and

Cₘₐₓ of desloratadine from about 2.371 ng/ml to about 3.705 mg/ml.

The ranges of bioequivalent pharmacokinetic parameters following administration of a chewable gel dosage form of the invention comprising about 10 mg loratadine under fasted conditions in Example 8 are set forth in Table C below.

TABLE C

| | AUC(0-t) | AUC(0-inf) | Cₘₐₓ |
|---|---|---|---|
| | hr*ng/ml | hr*ng/ml | ng/ml |
| Loratadine | 14.802 to 23.129 | 15.921 to 24.876 | 5.738 to 8.965 |
| Desloratadine | 31.841 to 49.751 | 34.729 to 54.264 | 2.810 to 4.390 |

Thus, in one embodiment of the invention, the administration of a chewable gel dosage form of the invention comprising about 10 mg loratadine under fasted conditions produces one or more of the following pharmacokinetic parameters:

AUC(0-t) of loratadine from about 14 hr*ng/ml to about 24 hr*ng/ml and AUC(0-inf) of loratadine from about 15 hr*ng/ml to about 25 hr*ng/ml;

Cₘₐₓ of loratadine from about 5 ng/ml to about 9 ng/ml;

AUC(0-t) of desloratadine from about 31 hr*ng/ml to about 50 hr*ng/ml and AUC(0-inf) of desloratadine from about 34 hr*ng/ml to about 55 hr*ng/ml; and

Cₘₐₓ of desloratadine from about 2 ng/ml to about 5 mg/ml.

In a further embodiment of the invention, the administration of a chewable gel dosage form comprising about 10 mg loratadine under fasted conditions produces one or more of the following pharmacokinetic parameters:

AUC(0-t) of loratadine from about 14.8 hr*ng/ml to about 23.2 hr*ng/ml and AUC(0-inf) of loratadine from about 15.9 hr*ng/ml to about 24.9 hr*ng/ml;

Cₘₐₓ of loratadine from about 5.7 ng/ml to about 9.0 ng/ml;

AUC(0-t) of desloratadine from about 31.8 hr*ng/ml to about 49.8 hr*ng/ml and AUC(0-inf) of desloratadine from about 34.7 hr*ng/ml to about 54.3 hr*ng/ml; and

Cₘₐₓ of desloratadine from about 2.8 ng/ml to about 4.4 mg/ml.

In another embodiment of the invention, the administration of a chewable gel dosage form of the invention comprising about 10 mg loratadine under fasted conditions produces one or more of the following pharmacokinetic parameters:

AUC(0-t) of loratadine from about 14.802 hr*ng/ml to about 23.129 hr*ng/ml and AUC(0-inf) of loratadine from about 15.921 hr*ng/ml to about 24.876 hr*ng/ml;

Cₘₐₓ of loratadine from about 5.738 ng/ml to about 8.965 ng/ml;

AUC(0-t) of desloratadine from about 31.841 hr*ng/ml to about 49.751 hr*ng/ml and AUC(0-inf) of desloratadine from about 34.729 hr*ng/ml to about 54.264 hr*ng/ml; and

Cₘₐₓ of desloratadine from about 2.810 ng/ml to about 4.390 mg/ml.

Alternatively, suitable treatment levels may include a range of pharmacokinetic parameters based on the First quartile and Third quartile of the AUC(0-t), AUC(0-inf) and Cₘₐₓ obtained following administration to a suitable subject population. The First quartile represents the median value for the half of the subject population below the median value for all subjects. The Third quartile represents the median value for the half of the subject population above the median value for all subjects.

Based on the pharmacokinetic characteristics observed from administration of a chewable gel dosage form of the invention containing about 10 mg under fed and fasted conditions set forth below in Examples 6 and 7, respectively, ranges of the First quartile to the Third quartile values are determined to provide a suitable treatment level. In another aspect, pharmacokinetic characteristics that are greater than the First quartile value provides a suitable treatment level.

The values of the first quartile and third quartile for loratadine following administration of the test formulation under fed conditions in Example 6 and fasted conditions in Example 7 are set forth in Table D below.

TABLE D

| | Fed Conditions | Fasted Conditions |
|---|---|---|
| Cₘₐₓ (ng/ml) | | |
| Median | 4.362 | 3.023 |
| First Quartile | 2.545 | 1.558 |
| Third Quartile | 6.339 | 6.000 |
| AUC(0-t) (hr*ng/ml) | | |
| Median | 13.127 | 8.219 |
| First Quartile | 8.190 | 4.951 |
| Third Quartile | 22.057 | 16.496 |
| AUC(0-inf) (hr*ng/ml) | | |
| Median | 13.550 | 8.480 |
| First Quartile | 8.557 | 5.009 |
| Third Quartile | 21.868 | 17.625 |

Thus, in an aspect of the invention, the administration of a chewable gel dosage form of the invention comprising about 10 mg loratadine under fed or fasted conditions produces one or more of the following pharmacokinetic parameters:

AUC(0-t) of loratadine from about 4.951 hr*ng/ml to about 22.057 hr*ng/ml and AUC(0-inf) of loratadine from about 5.009 hr*ng/ml to about 21.868 hr*ng/ml; and

Cₘₐₓ of loratadine from about 1.558 ng/ml to about 6.339 ng/ml.

Thus, in one embodiment of the invention, the administration of a chewable gel dosage form of the invention comprising about 10 mg loratadine under fed conditions produces one or more of the following pharmacokinetic parameters:

AUC(0-t) of loratadine from about 8.190 hr*ng/ml to about 22.057 hr*ng/ml and AUC(0-inf) of loratadine from about 8.557 hr*ng/ml to about 21.868 hr*ng/ml; and

Cₘₐₓ of loratadine from about 2.545 ng/ml to about 6.339 ng/ml.

In another embodiment of the invention, the administration of a chewable gel dosage form of the invention comprising about 10 mg loratadine under fasted conditions produces one or more of the following pharmacokinetic parameters:

AUC(0-t) of loratadine from about 4.951 hr*ng/ml to about 16.496 hr*ng/ml and AUC(0-inf) of loratadine from about 5.009 hr*ng/ml to about 17.625 hr*ng/ml; and

Cₘₐₓ of loratadine from about 1.558 ng/ml to about 6.000 mg/ml.

In alternative embodiment of the invention, the administration of a chewable gel dosage form comprising about 10 mg loratadine under fed conditions produces one or more of the following pharmacokinetic parameters:

AUC(0-t) of loratadine greater than about 8.190 hr*ng/ml and AUC(0-inf) of loratadine great than about 8.557 hr*ng/ml; and

Cₘₐₓ of loratadine great than about 2.545 ng/ml.

In another embodiment of the invention, the administration of a chewable gel dosage form of the invention comprising about 10 mg loratadine under fasted conditions produces one or more of the following pharmacokinetic parameters:

AUC(0-t) of loratadine greater than about 4.951 hr*ng/ml and AUC(0-inf) of loratadine great than about 5.009 hr*ng/ml; and

Cₘₐₓ of loratadine great than about 1.558 ng/ml.

In another embodiment of the invention, the pharmacokinetic parameters for loratadine and desloratadine are obtained from administration of the chewable gel dosage form of the invention containing 10 mg loratadine and a test product containing 10 mg loratadine. The formulation of the chewable gel dosage form of the invention can include the test product set forth below in Examples 6-8. The formulation of the reference product can include any dosage form other than a chewable gel formulation. For example, the formulation for the reference product may be a liquid capsule dosage form with caprylic/capric glycerides as an excipient. Alternatively, the reference product may be Claritin® (loratadine) Liqui-Gels® 10 mg capsule described below in Examples 6 and 7. The chewable gel dosage form of the invention and test product may be administered under fasted or fed conditions as described herein.

In some embodiments, the ratio of the median Cₘₐₓ of the chewable dosage form of the invention comprising about 10 mg loratadine to the reference product when administered under fed or fasted conditions is from about 1.1 to about 2.0, for example, about 1.2 to 1.6, about 1.2 to about 1.5, about 1.2 to about 1.3, or about 1.4 to about 1.5.

In some embodiments, the ratio of median AUC(0-t) or median AUC(0-inf) of the chewable dosage form of the invention comprising about 10 mg loratadine to the reference product when administered under fed or fasted conditions is from about 1.1 to about 2.5, for example, about 1.2 to about 2.1, about 1.2 to about 2.0, about 1.2 to about 1.3, or about 1.7 to about 2.0.

In other embodiments, the ratio of median Cₘₐₓ of the chewable dosage form of the invention comprising about 10 mg loratadine to the reference product when administered under fed conditions is from about 1.1 to about 1.6, for example about 1.2 to 1.5, or about 1.2 to about 1.3, or about 1.4 to about 1.5.

In other embodiments, the ratio of median AUC(0-t) or median AUC(0-inf) of the chewable dosage form of the invention comprising about 10 mg loratadine to the reference product when administered under fed conditions is from about 1.1 to about 1.6, for example about 1.2 to 1.5, about 1.2 to about 1.3, or about 1.4 to about 1.5.

In further embodiments, the ratio of median Cₘₐₓ of the chewable dosage form of the invention comprising about 10 mg loratadine to the reference product when administered under fasted conditions is from about 1.1 to about 1.6, for example about 1.2 to 1.5, about 1.3 to about 1.5, or about 1.4 to about 1.5.

In further embodiments, the ratio of median AUC(0-t) or median AUC(0-inf) of the chewable dosage form of the invention comprising about 10 mg loratadine to the reference product when administered under fasted conditions is from about 1.4 to about 2.5, for example about 1.6 to 2.1, about 1.7 to about 2.0, about 1.7 to about 1.8, or about 1.9 to about 2.0.

In some embodiments, the ratio of the Cₘₐₓ for the first quartile of the chewable dosage form of the invention to the reference product when administered under fed or fasted conditions is from about 1.1 to about 2.0, for example, about 1.2 to 1.7, about 1.3 to about 1.6, or about 1.5 to about 1.6.

In some embodiments, the ratio of AUC(0-t) or AUC(0-inf) for the first quartile of the chewable dosage form of the invention comprising about 10 mg loratadine to the reference product when administered under fed or fasted conditions is from about 1.1 to about 2.5, for example, about 1.2 to about 2.3, about 1.3 to about 2.2, about 1.3 to about 1.4, or about 2.1 to about 2.2.

In other embodiments, the ratio of Cₘₐₓ for the first quartile of the chewable dosage form of the invention comprising about 10 mg loratadine to the reference product when administered under fed conditions is from about 1.1 to about 1.8, for example about 1.2 to 1.7, about 1.3 to about 1.6, or about 1.5 to about 1.6.

In other embodiments, the ratio of AUC(0-t) or AUC(0-inf) for the first quartile of the chewable dosage form of the invention comprising about 10 mg loratadine to the reference product when administered under fed conditions is from about 1.1 to about 1.8, for example about 1.2 to 1.6, or about 1.2 to about 1.4, or about 1.3 to about 1.4.

In further embodiments, the ratio of Cₘₐₓ for the first quartile of the chewable dosage form of the invention comprising about 10 mg loratadine to the reference product when administered under fasted conditions is from about 1.1 to about 1.8, for example about 1.2 to 1.7, about 1.3 to about 1.6, or about 1.5 to about 1.6.

In further embodiments, the ratio of AUC(0-t) or AUC(0-inf) for the first quartile of the chewable dosage form of the invention comprising about 10 mg loratadine to the reference product when administered under fasted conditions is from about 1.1 to about 2.5, for example about 1.5 to 2.3, or about 1.8 to about 2.2, about 1.9 to about 2.2, or about 2.1 to about 2.2.

In some embodiments, the chewable gel dosage form of the invention exhibits a dissolution profile such that the composition releases at least about 5% of the active agent (or a pharmaceutically acceptable salt, ester, hydrate or solvate thereof), e.g., loratadine, within about 10 minutes in a 0.01M HCl solution at a temperature of 37.0±0.5° C. at 50 rpm. In some embodiments, the chewable gel dosage form of the invention exhibits a dissolution profile such that the composition releases at least about 10% of the active agent (or a pharmaceutically acceptable salt, ester, hydrate or solvate thereof), e.g., loratadine, within about 30 minutes under such conditions. In some embodiments, the chewable gel dosage form of the invention exhibits a dissolution profile such that the composition releases at least about 20%, at least about 50%, or at least about 70%, of the active agent (or a pharmaceutically acceptable salt, ester, hydrate or solvate thereof), e.g., loratadine, within about 10 minutes under such conditions. In some embodiments, the chewable gel dosage form of the invention exhibits a dissolution profile such that the composition releases at least about 30%, at least about 60%, or at least about 80%, of the active agent (or a pharmaceutically acceptable salt, ester, hydrate or solvate thereof), e.g., loratadine, within about 15 minutes under such conditions. In some embodiments, the chewable gel dosage form of the invention exhibits a dissolution profile such that the composition releases at least about 40%, at least about 70%, or at least about 80%, of the active agent (or a pharmaceutically acceptable salt, ester, hydrate or solvate thereof), e.g., loratadine, within about 20 minutes under such conditions. In some embodiments, the chewable gel dosage form of the invention exhibits a dissolution profile such that the composition releases at least about 50%, at least about 80%, or at least about 90%, of the active agent (or a pharmaceutically acceptable salt, ester, hydrate or solvate thereof), e.g., loratadine, within about 30 minutes under such conditions. In some embodiments, the chewable gel dosage form of the invention exhibits a dissolution profile such that the composition releases at least about 60%, at least about 80%, or at least about 90%, of the active agent (or a pharmaceutically acceptable salt, ester, hydrate or solvate thereof), e.g., loratadine, within about 40 minutes under such conditions. In some embodiments, the chewable gel dosage form of the invention exhibits a dissolution profile such that the composition releases at least about 80%, or at least about 90%, of the active agent (or a pharmaceutically acceptable salt, ester, hydrate or solvate thereof), e.g., loratadine, within about 60 minutes under such conditions.

In some embodiments, the chewable gel dosage form of the invention exhibits a dissolution profile such that the composition releases from about 1% to about 40%, from about 10% to about 40%, from about 20% to about 40%, or from about 25% to about 40%, of the active agent (or a pharmaceutically acceptable salt, ester, hydrate or solvate thereof), e.g., loratadine, within about 5 minutes in a 0.01M HCl solution at a temperature of 37.0±0.5° C. In some embodiments, the chewable gel dosage form of the invention exhibits a dissolution profile such that the composition releases from about 5% to about 70%, from about 20% to about 70%, from about 40% to about 70%, or from about 50% to about 70%, of the active agent (or a pharmaceutically acceptable salt, ester, hydrate or solvate thereof), e.g., loratadine, within about 10 minutes under such conditions. In some embodiments, the chewable gel dosage form of the invention exhibits a dissolution profile such that the composition releases from about 5% to about 90%, from about 30% to about 90%, from about 60% to about 90%, or from about 65% to about 90%, of the active agent (or a pharmaceutically acceptable salt, ester, hydrate or solvate thereof), e.g., loratadine, within about 15 minutes under such conditions. In some embodiments, the chewable gel dosage form of the invention exhibits a dissolution profile such that the composition releases from about 5% to about 90%, from about 40% to about 90%, or from about 70% to about 90%, of the active agent (or a pharmaceutically acceptable salt, ester, hydrate or solvate thereof), e.g., loratadine, within about 20 minutes under such conditions. In some embodiments, the chewable gel dosage form of the invention exhibits a dissolution profile such that the composition releases from about 10% to about 100%, from about 50% to about 100%, or from about 80% to about 100%, of the active agent (or a pharmaceutically acceptable salt, ester, hydrate or solvate thereof), e.g., loratadine, within about 30 minutes under such conditions. In some embodiments, the chewable gel dosage form of the invention exhibits a dissolution profile such that the composition releases from about 10% to about 100%, from about 60% to about 100%, or from about 80% to about 100%, of the active agent (or a pharmaceutically acceptable salt, ester, hydrate or solvate thereof), e.g., loratadine, within about 40 minutes under such conditions. In some embodiments, the chewable gel dosage form of the invention exhibits a dissolution profile such that the composition releases at least about 80% of the active agent (or a pharmaceutically acceptable salt, ester, hydrate or solvate thereof), e.g., loratadine, or such that at least about 80% of the chewable gel dosage form is dissolved, within about 60 minutes under such conditions. The dissolution profile may be evaluated by placing a single chewable gel dosage form of the invention (e.g., having a total mass of about 5 g) in a vessel equipped with a two-paddle stirrer and containing about 900 mL of a suitable dissolution medium, e.g., 0.01M aqueous HCl, stirring at about 50 rpm at a temperature of about 37.0±0.5° C., withdrawing 5 mL aliquots of the dissolution medium at different time points, e.g., at 10 minutes, 15 minutes, 30 minutes, 45 minutes, 60 minutes, 75 minutes, and 90 minutes, filtering each aliquot through a suitable filter, e.g., a 0.2 μm Nylon filter, and determining the concentration of active agent (or pharmaceutically acceptable salt, ester, hydrate or solvate thereof), e.g., loratadine, in each aliquot, e.g., by HPLC analysis relative to a suitable reference standard using a suitable column, mobile phase, and detector.

The texture of the chewable gel dosage form of the invention may be expressed in terms of firmness (or hardness), gumminess (or cohesiveness), or a combination thereof. Firmness may be determined, for example, by measuring the resistance to deformation of the chewable gel dosage form as a function of weight, e.g., in grams, applied to a surface thereof, e.g., by squeezing the dosage form between two plates. Gumminess of the chewable gel dosage form of the invention may be determined, for example, by measuring ability of the dosage form to recover from deformation following application of weight to a surface thereof, e.g., as described herein for measuring firmness. Gumminess may be expressed in terms of an area under a curve for a chewable gel dosage form of a particular hardness based on measurements of recovery from deformation following application of weight as described herein.

In some embodiments, the texture of the chewable gel dosage form of the invention may be determined in the same procedure by measuring both firmness and gumminess, and optionally repeating the procedure, e.g., a second time, on the same dosage form. For example, the texture of the chewable gel dosage form of the invention may be determined by measuring, in the same procedure, firmness, for example, resistance to deformation as a function of weight, e.g., in grams, applied to a surface thereof, e.g., by squeezing the dosage form between two plates, and gumminess, for example, extent of recovery from deformation following firmness measurement, and optionally repeating the procedure, e.g., a second time, on the same dosage form. Firmness and/or gumminess may be measured using a suitable texture analyzer, for example, a commercially available texture analyzer designed to measure firmness, gumminess, and/or stickiness for chewable gel confection products.

In some embodiments, the chewable gel dosage form of the invention has a firmness of from about 100 g to about 300 g, e.g., from about 100 g to about 250 g, e.g., from about 150 g to about 300 g, e.g., from about 150 g to about 250 g, e.g., from about 140 g to about 250 g. In some embodiments, the chewable gel dosage form of the invention has a gumminess of from about 1000 to about 2000, e.g., from about 1000 to about 1900, e.g., from about 1000 to about 1800, e.g., from about 1000 to about 1700, e.g., from about 1000 to about 1600. In some embodiments, the chewable gel dosage form of the invention has a firmness of from about 100 g to about 300 g, e.g., from about 100 g to about 250 g, e.g., from about 150 g to about 300 g, e.g., from about 150 g to about 250 g, e.g., from about 140 g to about 250 g, and a gumminess of from about 1000 to about 2000, e.g., from about 1000 to about 1900, e.g., from about 1000 to about 1800, e.g., from about 1000 to about 1700, e.g., from about 1000 to about 1600.

The following examples further illustrate the invention but, of course, should not be construed as in any way limiting its scope.

### Example 1

This example demonstrates a method of preparing a sugar-free chewable gel dosage form in accordance with an embodiment of the invention. The components of an exemplary formulation used in the manufacture of the chewable dosage form are listed below in Table 1A.

TABLE 1A

| | Formula |
|---|---|
| Ingredient | % by weight |
| Pectin | 2.68 |
| Maltitol (syrup) | 53.77 |
| Xylitol (powder) | 15.12 |
| Sorbitol (powder) | 5.89 |
| Sodium citrate | 0.20 |
| Citric Acid (50/50 solution) (dry basis) | 0.72 |
| Water | 18.99 |
| Glycerin USP | 1.95 |
| Loratadine | 0.20 |
| Tween 80 | 0.01 |
| FD&C Red #40 Solution | 0.19 |
| Cherry Flavor FFS (223G12) | 0.28 |

A primary blend is prepared that contains maltitol syrup, xylitol, sorbitol, sodium citrate, pectin and water. The primary blend is cooked to yield a Brix value of about 82°. A secondary blend is prepared that contains loratadine, glycerin, water, tween 80 (polysorbate 80), colorants and flavorants. An acid solution is prepared using citric acid.

The secondary blend and acid solution are combined with the primary blend to form the final blend. The final blend is mixed thoroughly to yield a Brix value of about 80° to about 82°. The final blend is transferred to a depositor hopper. From the depositor hopper, individual chewable gel dosage forms are prepared by depositing the final blend into pre-formed plastic molds. The size of each unit dosage is preferably selected to contain 10 mg of loratadine.

### Example 2

This example demonstrates a method of preparing a sugar-free chewable gel dosage form in accordance with an embodiment of the invention. The components of an exemplary formulation used in the manufacture of the chewable gel dosage form are listed below in Table 2A.

TABLE 2A

| | Formula |
|---|---|
| Ingredient | % by weight |
| Gelatin 275 Bloom Pig Skin | 7.56 |
| Maltitol (syrup) | 68.5 |
| Citric Acid (50/50 solution) (dry basis) | 0.82 |
| Water | 20.36 |
| Glycerin USP | 2.04 |
| Loratadine | 0.20 |
| Tween 80 | 0.02 |
| FD&C Red #40 Solution | 0.20 |
| Cherry Flavor FFS (223G12) | 0.30 |

A primary blend is prepared that contains maltitol syrup, gelatin and water. The primary blend is cooked to yield a Brix value of about 80°. A secondary blend is prepared that contains loratadine, glycerin, water, tween 80 (polysorbate 80), colorants and flavorants. An acid solution is prepared using citric acid.

The secondary blend and acid solution are combined with the primary blend to form the final blend. The final blend is mixed thoroughly to yield a Brix value of about 79° to about 81°. The final blend is transferred to a depositor hopper. From the depositor hopper, individual chewable gel dosage forms are prepared by depositing the final blend into pre-formed plastic molds. The size of each unit dosage is preferably selected to contain 10 mg of loratadine.

### Example 3

This example demonstrates a method of preparing a chewable gel dosage form in accordance with an embodiment of the invention. The components of an exemplary formulation used in the manufacture of the chewable gel dosage form are listed below in Table 3A.

TABLE 3A

| | Formula |
|---|---|
| Ingredient | % by weight |
| Sugar | 36.38 |
| Corn Syrup 63DE | 30.12 |
| Modified Food Starch (high amylose) | 10.24 |
| Sodium Citrate | 0.07 |
| Citric Acid (50/50 solution) (dry basis) | 0.80 |
| Water | 19.70 |
| Glycerin USP | 1.99 |
| Loratadine | 0.20 |
| Tween 80 | 0.02 |
| FD&C Red #40 Solution | 0.19 |
| Cherry Flavor FFS (223G12) | 0.29 |

A primary blend is prepared that contains corn syrup, sugar, modified food starch, sodium citrate and water. The primary blend is cooked to yield a Brix value of about 79°. A secondary blend is prepared that contains loratadine, glycerin, water, tween 80 (polysorbate 80), colorants and flavorants. An acid solution is prepared using citric acid.

The secondary blend and acid solution are combined with the primary blend to form the final blend. The final blend is mixed thoroughly to yield a Brix value of about 80° to about 82°. The final blend is transferred to a depositor hopper. From the depositor hopper, individual chewable gel dosage forms are prepared by depositing the final blend into pre-formed plastic molds. The size of each unit dosage is preferably selected to contain 10 mg of loratadine.

### Example 4

This example demonstrates a method of preparing a chewable gel dosage form in accordance with an embodiment of the invention. The components of an exemplary formulation used in the manufacture of the chewable gel dosage form are listed below in Table 4A.

TABLE 4A

| | Formula |
|---|---|
| Ingredient | % by weight |
| Agar Ticagel ® Nat GC-581 B | 2.91 |
| Sugar | 28.61 |
| Corn Syrup 43DE | 44.30 |
| Citric Acid (50/50 solution) (dry basis) | 0.79 |
| Water | 20.87 |
| Glycerin USP | 1.98 |
| Loratadine | 0.20 |
| Tween 80 | 0.02 |
| FD&C Red #40 Solution | 0.04 |
| Cherry Flavor FFS (223G12) | 0.28 |

A primary blend is prepared that contains sugar, corn syrup, agar and water. The primary blend is cooked to yield a Brix value of about 78°. A secondary blend is prepared that contains loratadine, glycerin, water, tween 80 (polysorbate 80), colorants and flavorants. An acid solution is prepared using citric acid.

The secondary blend and acid solution are combined with the primary blend to form the final blend. The final blend is mixed thoroughly to yield a Brix value of about 76° to about 79°. The final blend is transferred to a depositor hopper. From the depositor hopper, individual chewable gel dosage forms are prepared by depositing the final blend into pre-formed plastic molds. The size of each unit dosage is preferably selected to contain 10 mg of loratadine.

### Example 5

This example demonstrates a method of preparing a chewable gel dosage form in accordance with an embodiment of the invention. The components of an exemplary formulation used in the manufacture of the chewable gel dosage form are listed below in Table 5A.

TABLE 5A

| | Formula |
|---|---|
| Ingredient | % by weight |
| Glucose Syrup 54DE High-Maltose | 56.30 |
| Carrageenan Genutine ® Type 310-C | 2.53 |
| Sodium Citrate | 0.49 |
| Sugar | 16.6 |
| Citric Acid (50/50 solution) (dry basis) | 0.49 |
| Water | 20.99 |
| Glycerin USP | 1.95 |
| Loratadine | 0.20 |
| Tween 80 | 0.03 |
| FD&C Red #40 Solution | 0.13 |
| Cherry Flavor FFS (223G12) | 0.29 |

A primary blend is prepared that contains high-maltose corn syrup, sugar, carrageenan, sodium citrate and water. The primary blend is cooked to yield a Brix value of about 79°. A secondary blend is prepared that contains loratadine, glycerin, water, tween 80 (polysorbate 80), colorants and flavorants. An acid solution is prepared using citric acid.

The secondary blend and acid solution are combined with the primary blend to form the final blend. The final blend is mixed thoroughly to yield a Brix value of about 78° to about 80°. The final blend is transferred to a depositor hopper. From the depositor hopper, individual chewable gel dosage forms are prepared by depositing the final blend into pre-formed plastic molds. The size of each unit dosage is preferably selected to contain 10 mg of loratadine.

### Example 6

The bioavailability of about 10 mg loratadine in a chewable gel dosage form as compared to a reference formulation was evaluated when administered to human subjects with food.

An open label, randomized, two-treatment, three-sequence, three-period, partial replicate, cross-over, single-dose study with pharmacokinetic endpoints was carried out in healthy adult human subjects under fed conditions.

The two study drug treatments were as follows:

Test Product (T)—chewable gel dosage form containing about 10 mg loratadine prepared using the base, loratadine API pre-mix, and gelation solution as set forth in Tables 6A, 6B and 6C.

TABLE 6A

| Component in Base | Amount (quantity per unit) |
|---|---|
| Purified Water | 785.80 |
| Sodium Citrate Dihydrate | 6.62 |
| Citrus Pectin 200 | 98.22 |
| Polyglycitol Syrup | 2537.14 |
| Sugar | 2026.21 |

TABLE 6B

| Component in API Pre-Mix | Amount (quantity per unit) |
|---|---|
| Purified Water | 72.89 |
| Glycerin | 97.36 |
| Loratadine | 10.00 |
| Red Color | 9.54 |
| Tween 80 | 0.63 |
| Cherry Flavor | 13.81 |

TABLE 6C

| Component in Gelation Solution | Amount (quantity per unit) |
|---|---|
| Purified Water | 25.00 |
| Citric Acid Anhydrous | 25.00 |

The base is heated and combined with the API solution and gelation solution. During the manufacture, water is removed to reach 85% solids content for the final formulation prior to dispensing into individual dosage forms.

Reference Product (R)—Claritin® (loratadine) Liqui-Gels® 10 mg capsule that contains caprylic/capric glycerides, FD&C blue no. 1, gelatin, glycerin, pharmaceutical ink, polysorbate 80, povidone, purified water, and sorbitol.

For the test product (T), after an overnight fasting of at least 10.0 hours and exactly 30 minutes after serving of a high-fat, high-calorie breakfast, a single oral dose (one chewable gel dosage form containing about 10 mg loratadine) was administered without drinking water at room temperature. Subjects were asked to chew the test product, but not to spit or swallow it as a whole. After the chewable gel dosage form was completely chewed, the subject swallowed the remains without water.

For the reference product (R), after an overnight fasting of at least 10.0 hours and exactly 30 minutes after serving of a high-fat, high-calorie breakfast, a single oral dose (one Claritin® (loratadine) Liqui-Gels® 10 mg capsule) was administered with approximately 240 mL of drinking water at room temperature. The administration of the reference product (R) was repeated.

Blood samples (6 mL) were collected at various time points for analysis. A pre-dose blood sample was collected within one hour before dosing. The post-does blood samples were collected at the following times after dosing (in hours): 0.17, 0.33, 0.50, 0.67, 0.83, 1.00, 1.25, 1.50, 1.75, 2.00, 2.75, 3.00, 3.50, 4.00, 5.00, 6.00, 8.00, 10.00, 12.00, 16.00, 24.00, 26.00, 48.00, 72.00, 96.00 and 120.00 hours. Dosing of the reference product was repeated.

The blood plasma samples were analyzed to determine the content of loratadine and descarboethoxyloratadine. Descarboethoxyloratadine is an active metabolite of loratadine that is also known as desloratadine. Statistical analysis was carried out using standard techniques to determine loratadine and desloratadine concentration versus time curves (AUC) and peak concentrations (Cₘₐₓ).

The results of the untransformed data for loratadine are set forth in Tables 6D and 6E. The results of log-transformed data for loratadine are set forth in Table 6F.

TABLE 6D

| | Test Product (T) | Reference Product (R) |
|---|---|---|
| | (N = 46) | (N = 92) |
| Parameter | Mean +/− SD | Mean +/− SD |
| (unit) | CV (%) | CV (%) |
| Cₘₐₓ (ng/mL) | 5.429720 ± 4.5074163 | 5.357188 ± 5.9584034 |
| | (83.0) | (111.2) |
| AUC(0-t) | 18.081912 ± 16.6581896 | 15.630008 ± 15.2815664 |
| (hr*ng/mL) | (92.1) | (97.8) |
| AUC(0-inf) | 18.660547 ± 18.3352160 | 16.193945 ± 15.9684332 |
| (hr*ng/mL) | (98.3) | (98.6) |

TABLE 6E

| | Test Product | Reference Product | Ratio |
|---|---|---|---|
| Parameter (unit) | (T) | (R) | (T/R) |
| Cₘₐₓ (ng/mL) | | | |
| Median | 4.362 | 3.533 | 1.235 |
| First Quartile | 2.545 | 1.656 | 1.537 |
| Third Quartile | 6.339 | 6.402 | 0.990 |
| AUC(0-t) (hr*ng/mL) | | | |
| Median | 13.127 | 10.463 | 1.255 |
| First Quartile | 8.190 | 6.004 | 1.364 |
| Third Quartile | 22.057 | 20.675 | 1.067 |
| AUC(0-inf) (hr*ng/mL) | | | |
| Median | 13.550 | 10.506 | 1.290 |
| First Quartile | 8.557 | 6.154 | 1.390 |
| Third Quartile | 21.868 | 21.897 | 0.999 |

TABLE 6F

| | Geometric | Geometric | | 90% |
|---|---|---|---|---|
| | Mean | Mean | T/R Ratio | Confidence |
| Parameters | Test (T) | Reference (R) | (%) | Interval |
| Cₘₐₓ | 4.174 | 3.417 | 122.21 | NA |
| AUC(0-t) | 13.751 | 10.814 | 127.16 | 116.08-139.30 |
| AUC(0-inf)* | 14.705 | 11.179 | 131.54 | 119.38-144.94 |

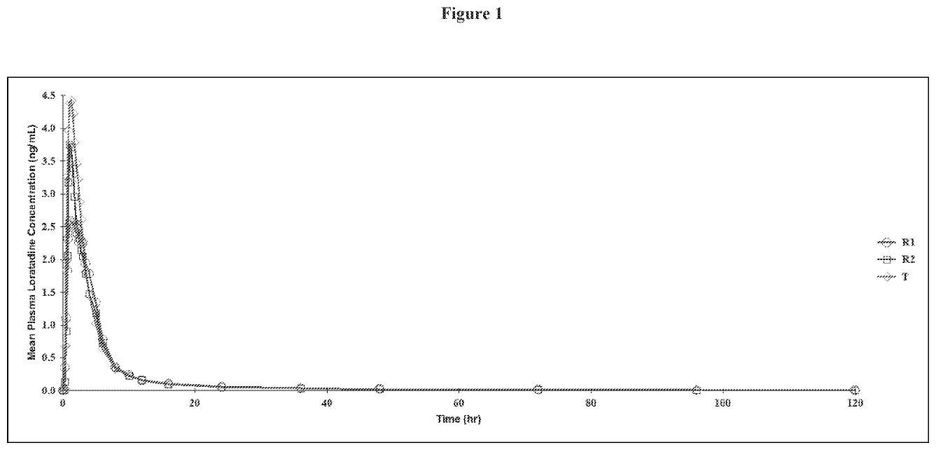

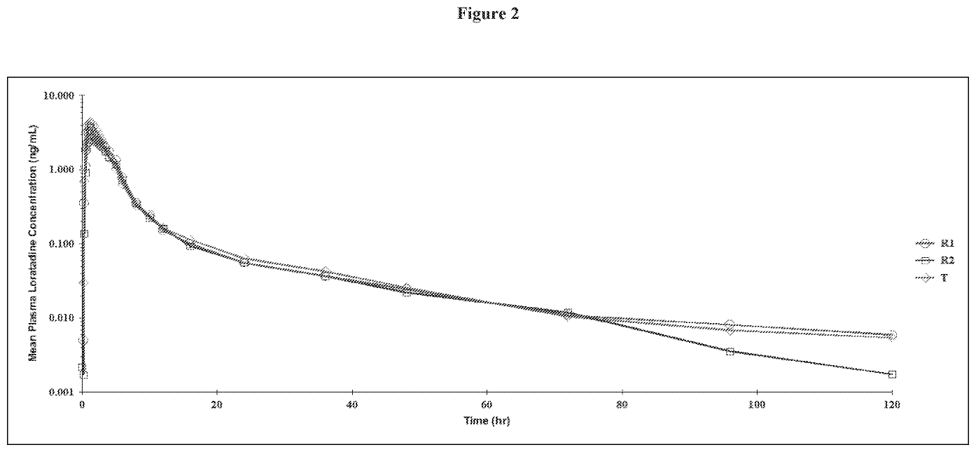

A linear plot of mean plasma loratadine concentrations v. time under this fed study is set forth in FIG. 1. A semi-log plot of mean plasma loratadine concentrations v. time under this fed study is set forth in FIG. 2.

The results of the untransformed and log-transformed data for desloratadine are set forth in Tables 6G and 6H, respectively.

TABLE 6G

| | Test Product (T) | Reference Product (R) |
|---|---|---|
| | (N = 44) | (N = 88) |
| Parameter | Mean +/− SD | Mean +/− SD |
| (unit) | CV (%) | CV (%) |
| Cₘₐₓ (ng/mL) | 3.410591 ± 1.1708570 | 3.674364 ± 1.6037739 |
| | (34.3) | (43.6) |
| AUC(0-t) | 63.594149 ± 48.0135964 | 63.566612 ± 45.7955088 |
| (hr*ng/mL) | (75.5) | (72.0) |
| AUC(0-inf) | 77.852702 ± 86.3585138 | 78.349300 ± 82.9011593 |
| (hr*ng/mL) | (110.9) | (105.8) |

TABLE 6H

| | Geometric | Geometric | | 90% |
|---|---|---|---|---|
| | Mean | Mean | T/R Ratio | Confidence |
| Parameters | Test (T) | Reference (R) | (%) | Interval |
| Cₘₐₓ | 3.246 | 3.407 | 95.27 | 88.53-102.53 |
| AUC(0-t) | 53.221 | 53.507 | 99.47 | 95.67-103.42 |
| AUC(0-inf)* | 58.213 | 58.861 | 98.90 | 95.08-102.87 |

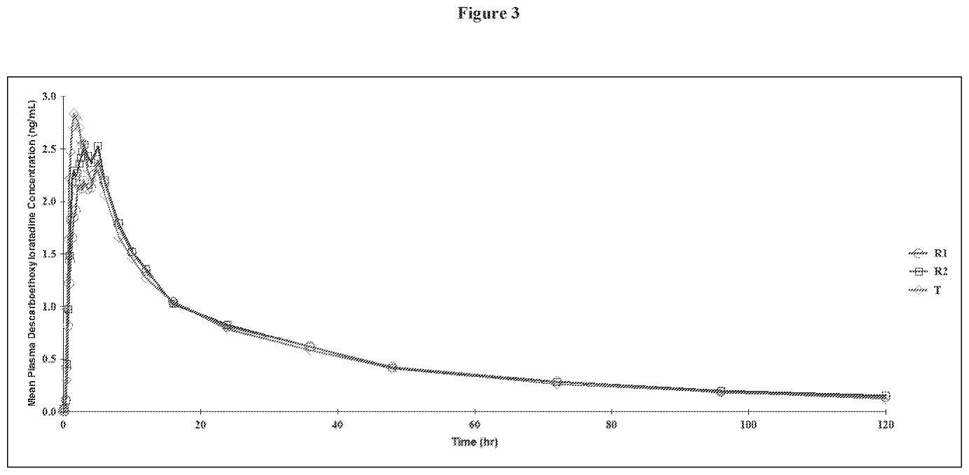

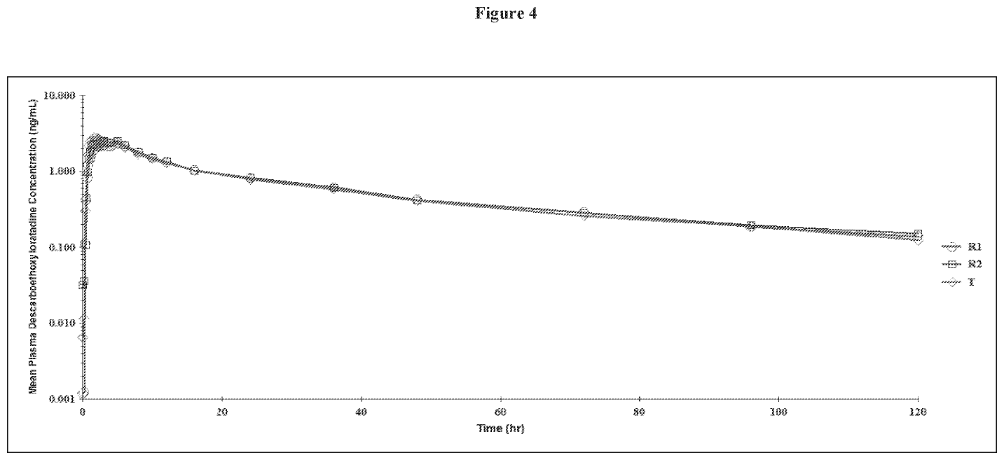

A linear plot of mean plasma desloratadine concentrations v. time under this fed study is set forth in FIG. 3. A semi-log plot of mean plasma desloratadine concentrations v. time under this fed study is set forth in FIG. 4.

### Example 7

The bioavailability of about 10 mg loratadine in a chewable gel dosage form as compared to a reference formulation was evaluated when administered to human subjects without food.

An open label, randomized, two-treatment, three-sequence, three-period, partial replicate, cross-over, single-dose study with pharmacokinetic endpoints was carried out in healthy adult human subjects under fasting conditions.

For the test product (T) (Example 6), after an overnight fast of at least 10 hours, a single oral dose (one chewable gel dosage form containing about 10 mg loratadine) was administered without drinking water at room temperature. Subjects were asked to chew the test product, but not to spit or swallow it as a whole. After the chewable gel dosage form was completely chewed, the subject swallowed the remains without water.

For the reference product (R), after an overnight fasting of at least 10 hours, a single oral dose (one Claritin® (loratadine) Liqui-Gels® 10 mg capsule) was administered with approximately 240 mL of drinking water at room temperature.

The administration of the reference product (R) was repeated according to the following treatment sequence:

| Sequence | Period 1 | Period 2 | Period 3 |
|---|---|---|---|
| Sequence 1 | T | R | R |
| Sequence 2 | R | T | R |
| Sequence 3 | R | R | T |

The washout period was at least 21 days between each treatment schedule.

Blood samples (6 mL) were collected at various time points for analysis. A pre-dose blood sample was collected within one hour before dosing. The post-does blood samples were collected at the following times after dosing (in hours): 0.17, 0.33, 0.50, 0.67, 0.83, 1.00, 1.25, 1.50, 1.75, 2.00, 2.75, 3.00, 3.50, 4.00, 5.00, 6.00, 8.00, 10.00, 12.00, 16.00, 24.00, 26.00, 48.00, 72.00, 96.00 and 120.00 hours. Dosing of the reference product was repeated.

The blood plasma samples were analyzed to determine the content of loratadine and desloratadine. Statistical analysis was carried out using standard techniques to determine loratadine and desloratadine concentration versus time curves (AUC) and peak concentrations (Cₘₐₓ).

The results of the untransformed data for loratadine are set forth in Tables 7A and 7B. The results of log-transformed data for loratadine are set forth in Table 7C.

TABLE 7A

| | Test Product (T) | Reference Product (R) |
|---|---|---|
| | (N = 45) | (N = 90) |
| Parameter | Mean +/− SD | Mean +/− SD |
| (unit) | CV (%) | CV (%) |
| Cₘₐₓ (ng/mL) | 4.438329 ± 4.1548982 | 3.582302 ± 3.8545386 |
| | (93.6) | (107.6) |
| AUC(0-t) | 12.825748 ± 11.2816660 | 10.869603 ± 13.7941201 |
| (hr*ng/mL) | (88.0) | (126.9) |
| AUC(0-inf)^{#} | 13.272957 ± 12.1091485 | 15.4705661 ± 15.4705661 |
| (hr*ng/mL) | (91.2) | (133.1) |

^{#}N = 43(T), 86(R) where subject did not exhibit log linear relationship at terminal elimination phase

TABLE 7B

| | Test Product | Reference Product | Ratio |
|---|---|---|---|
| Parameter (unit) | (T) | (R) | (T/R) |
| Cₘₐₓ (ng/mL) | | | |
| Median | 3.023 | 2.033 | 1.487 |
| First Quartile | 1.558 | 1.016 | 1.533 |
| Third Quartile | 6.000 | 4.668 | 1.285 |
| AUC(0-t) (hr*ng/mL) | | | |
| Median | 8.219 | 4.781 | 1.719 |
| First Quartile | 4.951 | 2.312 | 2.141 |
| Third Quartile | 16.496 | 15.139 | 1.090 |
| AUC(0-inf) (hr*ng/mL) | | | |
| Median | 8.480 | 4.282 | 1.980 |
| First Quartile | 5.009 | 2.327 | 2.152 |
| Third Quartile | 17.625 | 14.818 | 1.189 |

TABLE 7C

| | Geometric | Geometric | T/R | 90% |
|---|---|---|---|---|
| | Mean | Mean | Ratio | Confidence |
| Parameters | Test (T) | Reference (R) | (%) | Interval |
| Cₘₐₓ | 3.116 | 2.159 | 144.04 | NA |
| AUC(0-t) | 9.179 | 5.449 | 168.47 | 147.42-192.53 |
| AUC(0-inf)* | 9.444 | 5.686 | 166.09 | 145.27-189.90 |

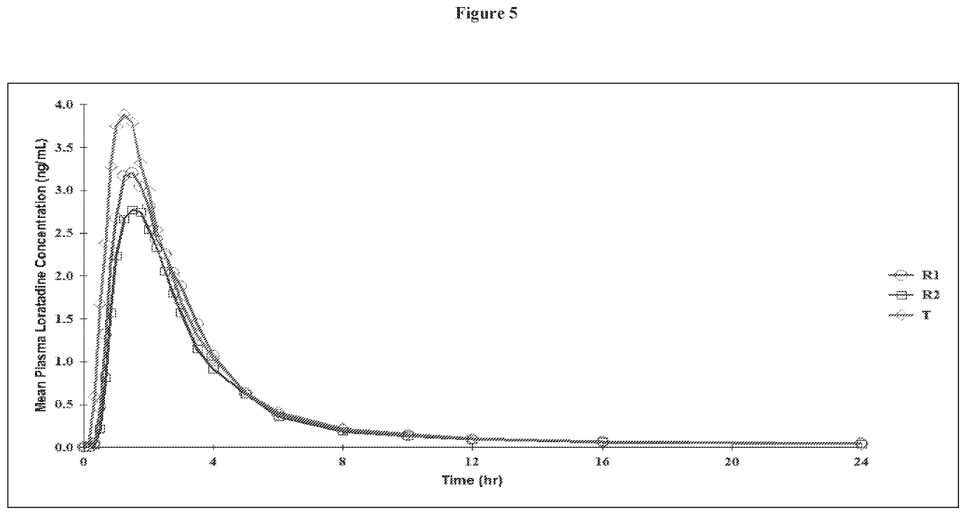

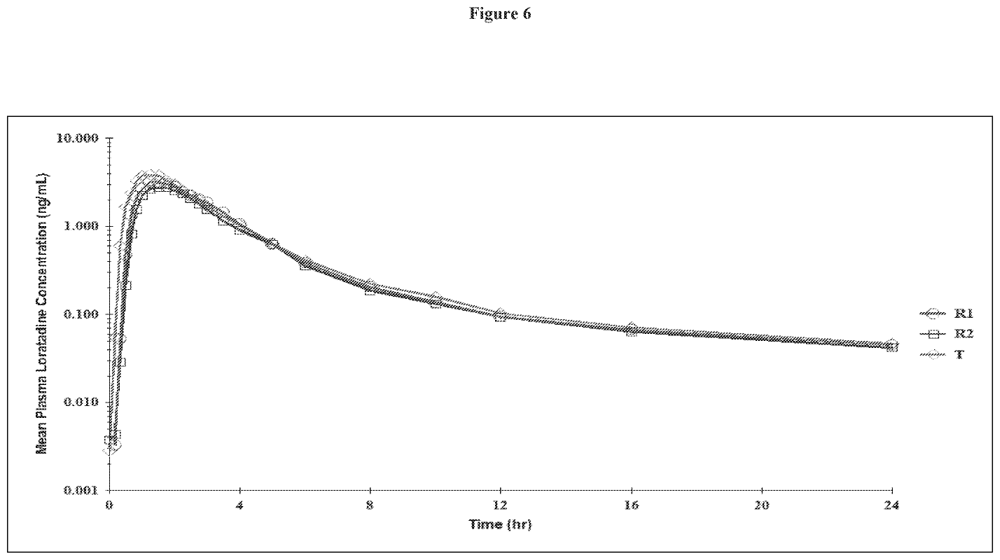

A linear plot of mean plasma loratadine concentrations v. time under this fed study is set forth in FIG. 5. A semi-log plot of mean plasma loratadine concentrations v. time under this fed study is set forth in FIG. 6.

The results of the untransformed and log-transformed data for desloratadine are set forth in Tables 7D and 7E, respectively.

TABLE 7D

| | Test Product (T) | Reference Product (R) |
|---|---|---|
| | (N = 44) | (N = 88) |
| Parameter (unit) | Mean +/− SD | Mean +/− SD |
| Cₘₐₓ (ng/mL) | 3.240114 ± 1.4126234 | 2.957568 ± 1.2604015 |
| | (43.6) | (42.6) |
| AUC(0-t) | 69.200724 ± 61.5694656 | 65.097665 ± 56.9472865 |
| (hr*ng/mL) | (89.0) | (87.5) |
| AUC(0-inf)^{#} | 84.679283 ± 101.4999910 | 77.359205 ± 86.8041723 |
| (hr*ng/mL) | (119.9) | (112.2) |

TABLE 7E

| | Geometric | Geometric | T/R | 90% |
|---|---|---|---|---|
| | Mean | Mean | Ratio | Confidence |
| Parameters | Test (T) | Reference (R) | (%) | Interval |
| Cₘₐₓ | 2.964 | 2.725 | 108.79 | 103.77 ± 114.06 |
| AUC(0-t) | 53.482 | 50.642 | 105.61 | 100.51 ± 110.96 |
| AUC(0-inf)* | 58.692 | 55.090 | 106.54 | 101.28 ± 112.08 |

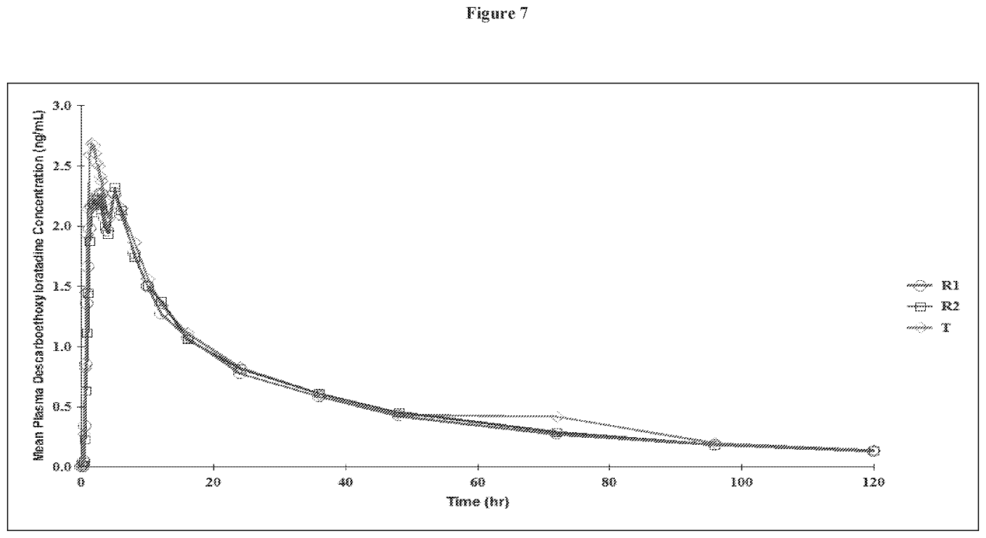

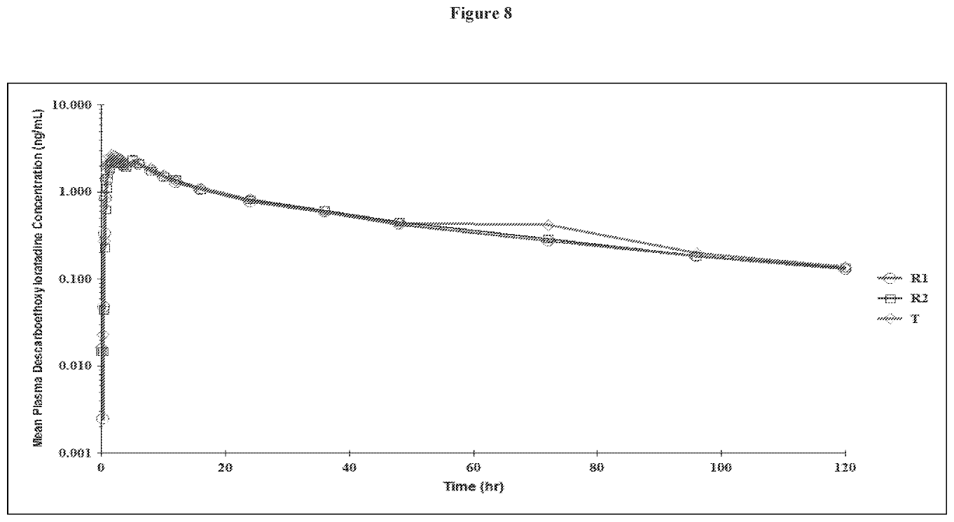

A linear plot of mean plasma desloratadine concentrations v. time under this fasted study is set forth in FIG. 7. A semi-log plot of mean plasma desloratadine concentrations v. time under this fasted study is set forth in FIG. 8.

### Example 8

The bioavailability of about 10 mg loratadine in a chewable gel dosage form as compared to a reference formulation was evaluated when administered to human subjects without food.

An open label, randomized, four-treatment, four-sequence, four-period, cross-over, single-dose study with pharmacokinetic endpoints was carried out in healthy adult human subjects under fasting conditions.

For the test product, after an overnight fast of at least 10 hours, a single oral dose (one chewable gel dosage form containing about 10 mg loratadine) was administered without drinking water at room temperature. Subjects were asked to chew the test product, but not to spit or swallow it as a whole. After the chewable gel dosage form was completely chewed, the subject swallowed the remains without water.

For the reference product 1 (R1), after an overnight fasting of at least 10 hours, a single oral dose (one Claritin (loratadine) 10 mg tablet) was administered with at least 240 mL of drinking water.

For the reference product 2 (R2), after an overnight fasting of at least 10 hours, a single oral dose of 10 mL (Claritin (loratadine) 5 mg/5 mL oral solution) was administered with at least 240 mL of drinking water.

For the reference product 3 (R3), after an overnight fasting of at least 10 hours, a single oral dose (one Claritin® (loratadine) Liqui-Gels® 10 mg capsule) was administered with at least 240 mL of drinking water.

Blood samples (6 mL) were collected at various time points for analysis. A pre-dose blood sample was collected within 60 minutes before dosing. The post-does blood samples were collected at the following times after dosing (in hours): 0.25, 0.50, 0.75, 1.00, 1.25, 1.50, 1.75, 2.00, 2.25, 2.50, 2.75, 3.00, 3.50, 4.00, 5.00, 6.00, 8.00, 10.00, 12.00, 16.00, 24.00, 48.00, 72.00, 96.00 and 120.00 hours.

The blood plasma samples were analyzed to determine the content of loratadine and desloratadine. Statistical analysis was carried out using standard techniques to determine loratadine and desloratadine concentration versus time curves (AUC) and peak concentrations (Cₘₐₓ).

The results of the untransformed and log-transformed data for loratadine are set forth in Tables 8A, 8B and 8C, respectively.

TABLE 8A

| | Test Product (T) | Reference Product 1 (R1) | Reference Product 2 (R2) | Reference Product 3 (R3) |
|---|---|---|---|---|
| | (N = 29) | (N = 29) | (N = 29) | (N = 29) |
| Parameter | Mean +/− SD | Mean +/− SD | Mean +/− SD | Mean +/− SD |
| (unit) | CV (%) | CV (%) | CV (%) | CV (%) |
| Cₘₐₓ (ng/mL) | 11.9331647 ± 11.68178750 | 9.3151928 ± 9.19534150 | 13.2321168 ± 11.19125347 | 9.4465174 ± 8.88048183 |
| | (97.89) | (98.71) | (84.58) | (94.01) |
| AUC(0-t) | 39.7806427 ± 53.98939559 | 37.6703859 ± 55.10343987 | 43.5941805 ± 52.99781695 | 38.6656877 ± 58.83918510 |
| (hr*ng/mL) | (135.72) | (146.28) | (121.57) | (152.17) |
| AUC(0-inf) | 42.9909034 ± 58.89920461 | 45.8660868 ± 62.82371803 | 47.1153790 ± 58.36891049 | 42.1631201 ± 65.03965573 |
| (hr*ng/mL) | (137.0) | (136.97) | (123.89) | (154.26) |

TABLE 8B

| | Geometric | Geometric Mean | Geometric Mean | Geometric Mean |
|---|---|---|---|---|
| Parameters | Mean Test (T) | Reference (R1) | Reference (R2) | Reference (R3) |
| Cₘₐₓ | 7.1719540 | 5.0844319 | 9.0933196 | 6.1894048 |
| AUC(0-t) | 18.5025219 | 15.0365250 | 24.7284738 | 16.9028045 |
| AUC(0-inf) | 19.9005922 | 19.0351095 | 26.2670416 | 18.4316058 |

TABLE 8C

| | | 90% | | 90% | | 90% |
|---|---|---|---|---|---|---|
| | | Confi- | | Confi- | | Confi- |
| | T/R1 | dence | T/R2 | dence | T/R3 | dence |
| | Ratio | Interval | Ratio | Interval | Ratio | Interval |
| Parameters | (%) | (T v. R1) | (%) | (T v. R2) | (%) | (T v. R3) |
| Cₘₐₓ | 141.06 | 118.51- | 78.87 | 66.29- | 115.87 | 97.35- |
| | | 167.89 | | 93.84 | | 137.92 |
| AUC | 123.05 | 108.42- | 74.82 | 65.94- | 109.46 | 96.45- |
| (0-t) | | 139.66 | | 84.90 | | 124.24 |
| AUC | 104.55 | 5.686 | 75.76 | 63.11- | 107.97 | 89.90- |
| (0-inf) | | | | 90.95 | | 129.67 |

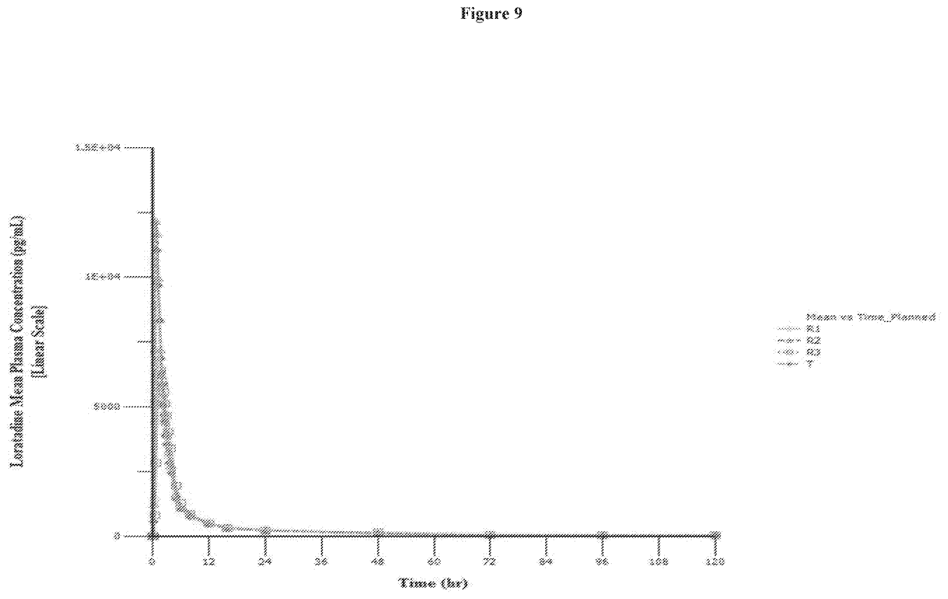

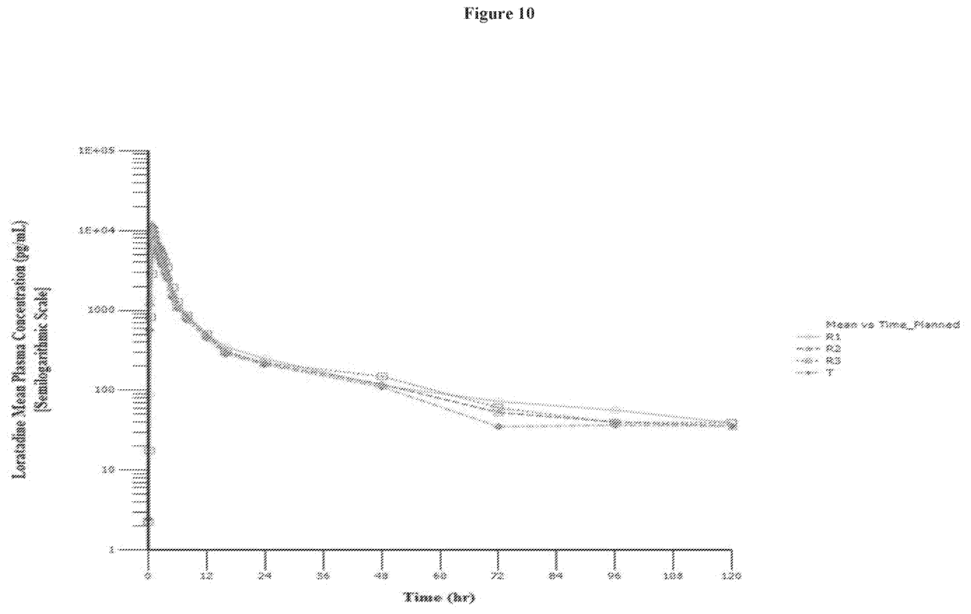

A linear plot of mean plasma loratadine concentrations v. time under this fed study is set forth in FIG. 9. A semi-log plot of mean plasma loratadine concentrations v. time under this fed study is set forth in FIG. 10.

The results of the untransformed and log-transformed data for desloratadine are set forth in Tables 8D, 8E and 8F, respectively.

TABLE 8D

| | Test | Reference | Reference | Reference |
|---|---|---|---|---|
| | Product (T) | Product 1 (R1) | Product 2 (R2) | Product 3 (R3) |
| | (N = 29) | (N = 29) | (N = 29) | (N = 29) |
| Parameter | Mean +/− SD | Mean +/− SD | Mean +/− SD | Mean +/− SD |
| (unit) | CV (%) | CV (%) | CV (%) | CV (%) |
| Cₘₐₓ (ng/mL) | 3.8774571 ± 16.8427548 | 3.4157431 ± 14.2256013 | 3.7943133 ± 12.3575477 | 3.9055973 ± 15.9441530 |
| | (43.44) | (41.65) | (32.57) | (40.82) |
| AUC(0-t) | 43.1016135 ± 20.98209477 | 42.9399101 ± 19.50896981 | 43.3395679 ± 20.88385961 | 45.4925163 ± 19.98058439 |
| (hr*ng/mL) | (48.68) | (45.43) | (48.19) | (43.92) |
| AUC(0-inf)^{#} | 46.7902953 ± 22.08436155 | 46.3764759 ± 21.02953059 | 46.8428210 ± 21.72307581 | 48.7561950 ± 21.16597676 |
| (hr*ng/mL) | (47.20) | (45.35) | (46.37) | (43.41) |

TABLE 8E

| | Geometric | Geometric Mean | Geometric Mean | Geometric Mean |
|---|---|---|---|---|
| Parameters | Mean Test (T) | Reference (R1) | Reference (R2) | Reference (R3) |
| Cₘₐₓ | 3.5120337 | 3.0732784 | 3.5313538 | 3.5927415 |
| AUC(0-t) | 39.8013373 | 39.9743219 | 39.9657300 | 42.2141790 |
| AUC(0-inf) | 43.4110637 | 43.5036288 | 43.6709626 | 45.5035533 |

TABLE 8F

| | | 90% | | 90% | | 90% |
|---|---|---|---|---|---|---|
| | | Confi- | | Confi- | | Confi- |
| | T/R1 | dence | T/R2 | dence | T/R3 | dence |
| | Ratio | Interval | Ratio | Interval | Ratio | Interval |
| Parameters | (%) | (T v. R1) | (%) | (T v. R2) | (%) | (T v. R3) |
| Cₘₐₓ | 114.28 | 103.97- | 99.45 | 90.51- | 97.75 | 88.94- |
| | | 125.60 | | 109.28 | | 107.44 |
| AUC | 99.57 | 93.48- | 99.59 | 93.51- | 94.28 | 88.52- |
| (0-t) | | 106.05 | | 106.06 | | 100.43 |
| AUC | 99.79 | 94.21- | 99.40 | 93.94- | 95.40 | 90.14- |
| (0-inf) | | 105.69 | | 105.19 | | 100.97 |

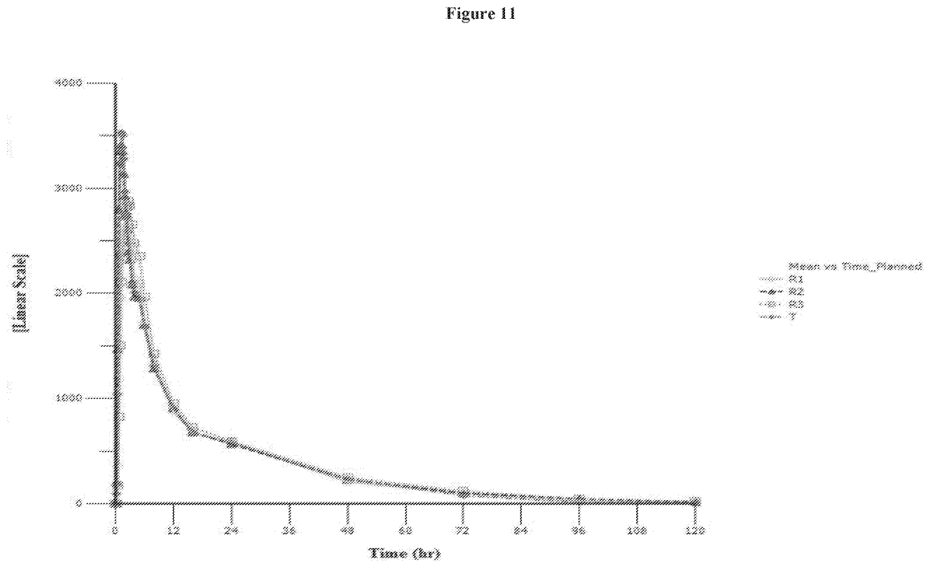

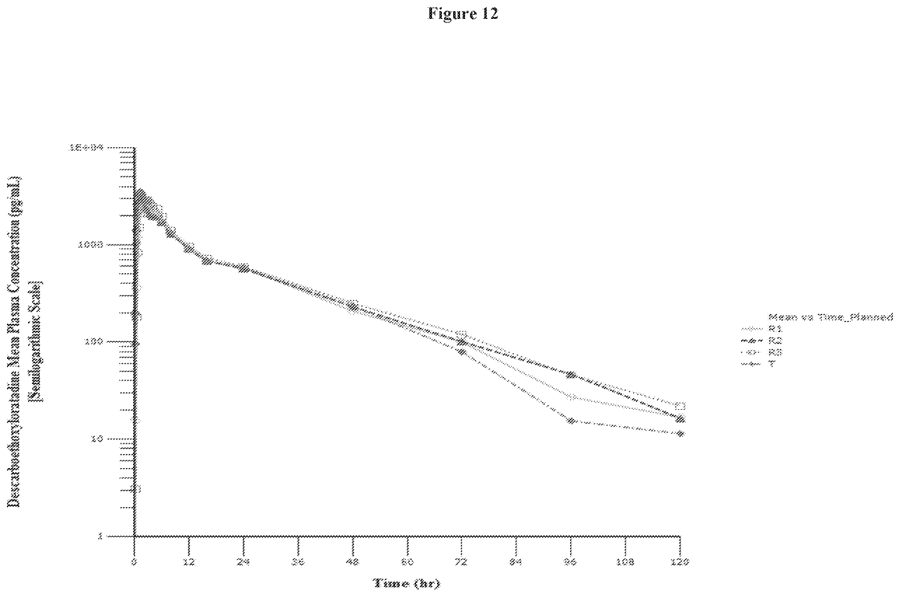

A linear plot of mean plasma desloratadine concentrations v. time under this fed study is set forth in FIG. 11. A semi-log plot of mean plasma desloratadine concentrations v. time under this fed study is set forth in FIG. 12.

### Example 9

This example describes the formulation components of a chewable gel dosage form of the invention, each having a mass of about 2.0 g.

TABLE 9A
Base Component After Cooking

| | mg/unit dosage | % of unit dosage |
|---|---|---|
| Ingredient | form | form |
| Water | 317.436 | 15.87% |
| Cargill Unipectine ™ 759 | 55.316 | 2.77% |
| CS MB HM Pectin | | |
| Sorbogem ® 1162 | 71.554 | 3.58% |
| Sodium Citrate Dihydrate | 4.846 | 0.24% |
| Roquette Lycasin ® 80/55 | 843.227 | 42.16% |
| HSH (Maltitol) Syrup | | |
| Xylitol Rochem-Futaste | 574.890 | 28.74% |
| Total | 1867.270 | 93.36% |

TABLE 9B
Active Component
(solids content about 39%)

| | mg/unit | % of unit |
|---|---|---|
| | dosage | dosage |
| Ingredient | form | form |
| GLYCERIN | 24.30 | 1.21% |
| 99.5% (KOSHER) | | |
| PURIFIED WATER | 43.50 | 2.18% |
| LORATADINE | 10.00 | 0.50% |
| TWEEN 80 | 0.63 | 0.03% |
| (POLYSORBATE 80) | | |
| N&A STRAWBERRY | 2.30 | 0.12% |
| FLV (30743955\001) | | |
| FD&C RED #40-4% SOLUTION | 10.00 | 0.50% |
| Total | 90.730 | 4.54% |

TABLE 9C
Citric Acid Component
(solids content about 50%)

| | mg/unit dosage | % of unit dosage |
|---|---|---|
| Ingredient | form | form |
| CITRIC ACID ANHYDROUS | 21.00 | 1.05% |
| FILTERED WATER | 21.00 | 1.05% |
| Total | 42.00 | 2.10% |

The components are combined to produce a chewable gel dosage form of the invention with a solids content of about 80%. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 10

This example describes the formulation components of a chewable gel dosage form of the invention each having a mass of about 2.0 g.

TABLE 10A
Base Component After Cooking
(initial solids content before cooking about 68%)

| | mg/unit dosage | % of unit dosage |
|---|---|---|
| Ingredient | form | form |
| Water | 317.436 | 15.87% |
| Cargill Unipectine ™ 759 | 55.316 | 2.77% |
| CS MB HM Pectin | | |
| Sorbogem ® 1162 | 178.886 | 8.94% |
| Sodium Citrate Dihydrate | 4.846 | 0.24% |
| Roquette Lycasin ® 80/55 | 850.873 | 42.54% |
| HSH (Maltitol) Syrup | | |
| Xylitol Rochem-Futaste | 459.912 | 23.00% |
| Total | 1867.270 | 93.36% |

TABLE 10B
Active Component
(solids content about 39%)

| | mg/unit dosage | % of unit dosage |
|---|---|---|
| Ingredient | form | form |
| GLYCERIN 99.5% | 24.30 | 1.21% |
| (KOSHER) | | |
| PURIFIED WATER | 43.50 | 2.18% |
| LORATADINE | 10.00 | 0.50% |
| TWEEN 80 | 0.63 | 0.03% |
| (POLYSORBATE 80) | | |
| N&A STRAWBERRY | 2.30 | 0.12% |
| FLV (30743955\001) | | |
| FD&C RED #40-4% SOLUTION | 10.00 | 0.50% |
| Total | 90.730 | 4.54% |

TABLE 10C
Citric Acid Component
(solids content about 50%)

| | mg/unit dosage | % of unit dosage |
|---|---|---|
| Ingredient | form | form |
| CITRIC ACID ANHYDROUS | 21.00 | 1.05% |
| FILTERED WATER | 21.00 | 1.05% |
| Total | 42.00 | 2.10% |

The components are combined to produce a chewable gel dosage form of the invention with a solids content of about 80%. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 11

This example describes the formulation components of a chewable gel dosage form of the invention each having a mass of about 2.0 g.

TABLE 11A
Base Component After Cooking
(initial solids content before cooking about 68%)

| | mg/unit dosage | % of unit dosage |
|---|---|---|
| Ingredient | form | form |
| Water | 317.436 | 15.87% |
| Cargill Unipectine ™ 759 | 55.316 | 2.77% |
| CS MB HM Pectin | | |
| Sorbogem ® 1162 | 143.109 | 7.16% |
| Sodium Citrate Dihydrate | 4.846 | 0.24% |
| Roquette Lycasin ® 80/55 | 817.664 | 40.88% |
| HSH (Maltitol) Syrup | | |
| Xylitol Rochem-Futaste | 528.899 | 26.44% |
| Total | 1867.270 | 93.36% |

TABLE 11B
Active Component
(solids content about 39%)

| | mg/unit dosage | % of unit dosage |
|---|---|---|
| Ingredient | form | form |
| GLYCERIN 99.5% (KOSHER) | 24.30 | 1.21% |
| PURIFIED WATER | 43.50 | 2.18% |
| LORATADINE | 10.00 | 0.50% |
| TWEEN 80 (POLYSORBATE 80) | 0.63 | 0.03% |
| N&A STRAWBERRY FLV | 2.30 | 0.12% |
| (30743955\001) | | |
| FD&C RED #40-4% SOLUTION | 10.00 | 0.50% |
| Total | 90.730 | 4.54% |

TABLE 11C
Citric Acid Component
(solids content about 50%)

| | mg/unit dosage | % of unit dosage |
|---|---|---|
| Ingredient | form | form |
| CITRIC ACID ANHYDROUS | 21.00 | 1.05% |
| FILTERED WATER | 21.00 | 1.05% |
| Total | 42.00 | 2.10% |

The components are combined to produce a chewable gel dosage form of the invention with a solids content of about 80%. The mass of each unit chewable gel dosage form is about 2.0 g. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 12

This example describes the formulation components of a chewable gel dosage form of the invention each having a mass of about 2.0 g.

TABLE 12A
Base Component After Cooking
(initial solids content before cooking about 69%)

| | mg/unit dosage | % of unit |
|---|---|---|
| Ingredient | form | dosage form |
| Water | 317.436 | 15.87% |
| Cargill Unipectine ™ 759 CS MB HM | 55.316 | 2.77% |
| Pectin | | |
| Sorbogem ® 1162 | 178.886 | 8.94% |
| Sodium Citrate Dihydrate | 4.846 | 0.24% |
| Roquette Lycasin ® 80/55 HSH Syrup | 735.895 | 36.79% |
| Xylitol Rochem- Futaste | 574.890 | 28.74% |
| Total | 1867.270 | 93.36% |

TABLE 12B
Active Component
(solids content about 39%)

| | mg/unit dosage | % of unit |
|---|---|---|
| Ingredient | form | dosage form |
| GLYCERIN 99.5% (KOSHER) | 24.30 | 1.21% |
| PURIFIED WATER | 43.50 | 2.18% |
| LORATADINE | 10.00 | 0.50% |
| TWEEN 80 (POLYSORBATE 80) | 0.63 | 0.03% |
| N&A STRAWBERRY FLV | 2.30 | 0.12% |
| (30743955\001) | | |
| FD&C RED #40-4% SOLUTION | 10.00 | 0.50% |
| Total | 90.730 | 4.54% |

TABLE 12C
Gelation Component
(solids content about 50%)

| | mg/unit dosage | % of unit dosage |
|---|---|---|
| Ingredient | form | form |
| CITRIC ACID ANHYDROUS | 21.00 | 1.05% |
| FILTERED WATER | 21.00 | 1.05% |
| Total | 42.00 | 2.10% |

The components are combined to produce a chewable gel dosage form of the invention with a solids content of about 80%. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 13

This example describes formulation components of a chewable gel dosage form of the invention, each unit dosage having a mass of about 5.0 g.

TABLE 13A
Base Component After Cooking
(initial solids content before cooking about 69%)

| | mg/unit | % of unit |
|---|---|---|
| Ingredient | dosage form | dosage form |
| Water | 870.709 | 17.41% |
| Cargill Unipectine ™ 759 CS MB HM | 126.782 | 2.54% |
| Pectin | | |
| Sorbitol SORBOGEM ® | 460.477 | 9.21% |
| Sodium Citrate Dihydrate | 5.162 | 0.10% |
| Roquette Lycasin ® 80/55 HSH Syrup | 2190.264 | 43.81% |
| Xylitol Powder XIVIA ® C | 1183.877 | 23.68% |

TABLE 13B
Active Component
(solids content about 54%)

| | mg/unit | % of unit |
|---|---|---|
| Ingredient | dosage form | dosage form |
| GLYCERIN 99.5% (KOSHER) | 24.30 | 0.49% |
| PURIFIED WATER | 32.45 | 0.65% |
| LORATADINE | 10.00 | 0.20% |
| TWEEN 80 (POLYSORBATE 80) | 0.63 | 0.01% |
| RED COLOR SOLUTION | 9.54 | 0.19% |
| CHERRY FLAVOR ARTIFICIAL | 13.81 | 0.28% |

TABLE 13C
Gelation Component

| | mg/unit | % of unit |
|---|---|---|
| Ingredient | dosage form | dosage form |
| CITRIC ACID ANHYDROUS | 36.00 | 0.72% |
| FILTERED WATER | 36.00 | 0.72% |

The components are combined to produce a chewable gel dosage form of the invention. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 14

This example describes formulation components of a chewable gel dosage form of the invention, each unit dosage having a mass of about 2.0 g.

TABLE 14A
Base Component After Cooking
(initial solids content before cooking about 69%)

| | mg/unit | % of unit |
|---|---|---|
| Ingredient | dosage form | dosage form |
| Water | 316.382 | 15.82% |
| Cargill Unipectine ™ 759 CS MB HM | 46.068 | 2.30% |
| Pectin | | |
| Sorbitol SORBOGEM ® | 167.319 | 8.37% |
| Sodium Citrate Dihydrate | 1.876 | 0.09% |
| Roquette Lycasin ® 80/55 HSH Syrup | 795.857 | 39.79% |
| Xylitol Powder XIVIA ® C | 430.175 | 21.51% |

TABLE 14B
Active Component
(solids content about 49%)

| | mg/unit | % of unit |
|---|---|---|
| Ingredient | dosage form | dosage form |
| GLYCERIN 99.5% (KOSHER) | 72.89 | 3.64% |
| PURIFIED WATER | 97.36 | 4.87% |
| LORATADINE | 10.00 | 0.50% |
| TWEEN 80 (POLYSORBATE 80) | 0.63 | 0.03% |
| RED COLOR SOLUTION | 9.54 | 0.48% |
| CHERRY FLAVOR ARTIFICIAL | 13.81 | 0.69% |
| POVIDONE | 8.09 | 0.40% |

TABLE 14C
Gelation Component

| | mg/unit | % of unit |
|---|---|---|
| Ingredient | dosage form | dosage form |
| CITRIC ACID ANHYDROUS | 15.00 | 0.75% |
| FILTERED WATER | 15.00 | 0.75% |

The components are combined to produce a chewable gel dosage form of the invention. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 15

This example describes formulation components of a chewable gel dosage form of the invention, each unit dosage having a mass of about 2.0 g.

TABLE 15A
Base Component After Cooking
(initial solids content before cooking about 68-69%)

| | mg/unit dosage | % of unit dosage |
|---|---|---|
| Ingredient | form | form |
| Water | 338.269 | 16.91% |
| Nittta N-Gel27 | 102.567 | 5.13% |
| Roquette Lycasin ® 80/55 HSH Syrup | 1438.435 | 71.92% |

TABLE 15B
Active Component
(solids content about 54%)

| | mg/unit dosage | % of unit dosage |
|---|---|---|
| Ingredient | form | form |
| GLYCERIN 99.5% (KOSHER) | 24.30 | 1.21% |
| PURIFIED WATER | 32.45 | 1.62% |
| LORATADINE | 10.00 | 0.50% |
| TWEEN 80 (POLYSORBATE 80) | 0.63 | 0.03% |
| RED COLOR SOLUTION | 9.54 | 0.48% |
| CHERRY FLAVOR ARTIFICIAL | 13.81 | 0.69% |

TABLE 15C
Gelation Component

| | mg/unit | % of unit |
|---|---|---|
| Ingredient | dosage form | dosage form |
| CITRIC ACID ANHYDROUS | 15.00 | 0.75% |
| FILTERED WATER | 15.00 | 0.75% |

The components are combined to produce a chewable gel dosage form of the invention. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 16

This example describes formulation components of a chewable gel dosage form of the invention, each unit dosage having a mass of about 2.0 g.

TABLE 16A
Base Component After Cooking
(initial solids content before cooking about 56%)

| | mg/unit | % of unit |
|---|---|---|
| Ingredient | dosage form | dosage form |
| Water | 343.669 | 17.18% |
| Tic Gums Agar RS-111 | 47.644 | 2.38% |
| Sorbogem ® 1162 | 182.283 | 9.11% |
| Roquette Lycasin ® 80/55 HSH Syrup | 867.030 | 43.35% |
| Xylitol (Xivia ® C) | 468.645 | 23.43% |

TABLE 16B
Active Component
(solids content about 54%)

| | mg/unit | % of unit |
|---|---|---|
| Ingredient | dosage form | dosage form |
| GLYCERIN 99.5% (KOSHER) | 24.30 | 1.21% |
| PURIFIED WATER | 32.45 | 1.62% |
| LORATADINE | 10.00 | 0.50% |
| TWEEN 80 (POLYSORBATE 80) | 0.63 | 0.03% |
| RED COLOR SOLUTION | 9.54 | 0.48% |
| CHERRY FLAVOR ARTIFICIAL | 13.81 | 0.69% |

The components are combined to produce a chewable gel dosage form of the invention. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

### Example 17

This example describes an exemplary chewable gel dosage form of the invention, which includes the following ingredients.

TABLE 17A
Formulation of Finished Dosage Form

| | Weight |
|---|---|
| Ingredient | Percent |
| Pectin | 2.50 |
| Maltitol Syrup | 43.90 |
| Xylitol | 29.93 |
| Sorbitol (dry) | 3.73 |
| Sodium citrate dihydrate | 0.22 |
| Citric Acid anhydrous | 0.79 |
| Water | 14.41 |
| Glycerin | 3.87 |
| Loratadine | 0.50 |
| Polysorbate 80 | 0.03 |
| FD&C Red 40 | 0.02 |
| Flavor Strawberry N&A | 0.12 |

An exemplary batch of the product is manufactured as follows.

TABLE 17B

| | Amount (kg) |
|---|---|
| Pectin Hydration Mixture | |
| Pectin | 45.8 |
| Sodium Citrate Dihydrate | 3.5 |
| Sorbitol, dry | 60.1 |
| Water | 320.6 |
| Total: | 430 |
| Pre-Cook Blend | |
| Hydrated pectin | 430 |
| Xylitol | 482.49 |
| Maltitol Syrup | 943.60 |
| Total: | 1856.09 |
| Post-Cook Blend | |
| Water | 219.5 |
| Pectin | 40.3 |
| Xylitol | 482.5 |
| Maltitol Syrup | 707.7 |
| Sodium Citrate Dihydrate | 3.5 |
| Sorbitol, dry | 60.1 |
| Total: | 1514 |
| Active Ingredient and Flavor Solution | |
| Glycerin | 62.39 |
| Loratadine | 8.061 |
| Polysorbate 80 | 0.508 |
| Flavor Strawberry N&A | 1.854 |
| FD&C Red 40 | 0.32 |
| Total: | 73.1 |
| Gelation Solution | |
| Citric acid, anhydrous | 12.78 |
| Water | 12.78 |
| Total: | 25.55 |
| Grand Total | 1612 |

The components are prepared, combined, and dispensed into suitable molds in accordance with Example 1 to produce a chewable gel dosage form of the invention. A 2 g unit dosage form will contain about 10 mg of loratadine. The chewable gel dosage form is packaged in suitable blister packaging, exhibits acceptable gel firmness, ejection force, adhesion, stickiness (wet and dry), gumminess, and swelling properties, and is stable for at least about 6 months at 40° C. and 75% RH.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for the temporary relief of symptoms due to hay fever and upper respiratory allergies, the method comprising administering to a subject in need thereof under fasted or fed conditions a chewable gel dosage form comprising about 10 mg loratadine or a pharmaceutically acceptable salt thereof and at least one gelling agent, wherein the dosage form provides an AUC(0-t) and an AUC(0-inf) of loratadine of at least about 3 ng*hr/mL in the subject under fasted or fed conditions, and has dissolution profile such that the dosage form releases from about 70% to about 90% of the loratadine or pharmaceutically acceptable salt thereof within about 20 minutes in a 0.01 M HCl solution at a temperature of 37.0±0.5° C.

2. The method of claim 1, wherein the dosage form provides an AUC(0-t) and an AUC(0-inf) of loratadine of from about 3 ng*hr/mL to about 25 ng*hr/mL in the subject under fasted or fed conditions.

3. The method of claim 1, wherein the dosage form provides a Cₘₐₓ of loratadine of at least about 2 ng/mL in the subject under fasted or fed conditions.

4. The method of claim 1, wherein the dosage form provides a Cₘₐₓ of loratadine of from about 2 ng/mL to about 10 ng/mL in the subject under fasted or fed conditions.

5. The method of claim 1, wherein the dosage form provides an AUC(0-t) and an AUC(0-inf) of desloratadine of at least about 25 ng*hr/mL in the subject under fasted or fed conditions.

6. The method of claim 1, wherein the dosage form provides an AUC(0-t) and an AUC(0-inf) of desloratadine of from about 25 ng*hr/mL to about 80 ng*hr/mL in the subject under fasted or fed conditions.

7. The method of claim 1, wherein the dosage form provides a Cₘₐₓ of desloratadine of at least about 1 ng/mL in the subject under fasted or fed conditions.

8. The method of claim 1, wherein the dosage form provides a Cₘₐₓ of desloratadine of from about 1 ng/mL to about 8 ng/mL in the subject under fasted or fed conditions.

9. The method of claim 1, comprising administering the dosage form to the subject under fasted conditions, wherein the dosage form provides an AUC(0-t) and an AUC(0-inf) of loratadine of at least about 5 ng*hr/mL in the subject under fasted conditions.

10. The method of claim 1, comprising administering the dosage form to the subject under fed conditions, wherein the dosage form provides an AUC(0-t) and an AUC(0-inf) of loratadine of at least about 10 ng*hr/mL in the subject under fed conditions.

11. The method of claim 10, wherein the dosage form provides an AUC(0-t) and an AUC(0-inf) of desloratadine of at least about 35 ng*hr/mL in the subject under fed condition.

12. The method of claim 10, wherein the dosage form provides a Cₘₐₓ of loratadine of at least about 3 ng/mL in the subject under fed conditions.

13. The method of claim 10, wherein the dosage form provides an AUC(0-t) and an AUC(0-inf) of desloratadine of at least about 40 ng*hr/mL in the subject under fed conditions.

14. The method of claim 10, wherein the dosage form provides a Cₘₐₓ of desloratadine of at least about 2 ng/mL in the subject under fed conditions.

15. The method of claim 9, wherein the dosage form provides an AUC(0-t) and an AUC(0-inf) of loratadine of at least about 7 ng*hr/mL and a Cₘₐₓ of desloratadine of at least about 2 ng/mL in the subject under fasted conditions.

16. The method of claim 9, wherein the dosage form provides an AUC(0-t) and an AUC(0-inf) of loratadine of at least about 14 ng*hr/mL and a Cₘₐₓ of loratadine of at least about 5 ng/mL in the subject under fasted conditions.

17. The method of claim 1, wherein the dosage form provides an AUC(0-t) and an AUC(0-inf) of loratadine of from about 5 ng*hr/mL to about 22 ng*hr/mL and a Cₘₐₓ of loratadine of from about 1.5 ng/mL to about 6.5 ng/mL in the subject under fasted or fed conditions.

18. The method of claim 1, wherein the ratio of the Cₘₐₓ of loratadine for the first quartile provided by the chewable gel dosage form relative to a liquid gel capsule containing about 10 mg of loratadine is from about 1.1 to about 2.0 in the subject under fasted or fed conditions.

19. The method of claim 1, wherein the ratio of the AUC(0-t) or AUC(0-inf) of loratadine for the first quartile provided by the chewable gel dosage form relative to a liquid gel capsule containing about 10 mg of loratadine is from about 1.1 to about 2.5 in the subject under fasted or fed conditions.

20. A chewable gel dosage form comprising loratadine or a pharmaceutically acceptable salt, ester, hydrate or solvate thereof, one or more polyols, and at least one gelling agent.

21. The chewable gel dosage form of claim 20, wherein the one or more polyols comprises maltitol, xylitol, sorbitol, or a combination thereof.

22. The chewable gel dosage form of claim 20, wherein the one or more polyols comprises maltitol, xylitol, and sorbitol.

23. The chewable gel dosage form of claim 22, wherein the maltitol comprises maltitol syrup.

24. The chewable gel dosage form of claim 22, wherein the gelling agent comprises pectin, carrageenan, agar, starch, gelatin, or a combination thereof.

25. The chewable gel dosage form of claim 20, wherein the gelling agent comprises pectin.

26. The chewable gel dosage form of claim 20, further comprising a surfactant.

27. The chewable gel dosage form of claim 26, wherein the surfactant comprises a polyoxyethylene sorbitan ester.

28. The chewable gel dosage form of claim 26, wherein the surfactant comprises polysorbate 80.

29. The chewable gel dosage form of claim 20, further comprising a buffer.

30. The chewable gel dosage form of claim 29, wherein the buffer comprises citric acid, sodium citrate, or a combination thereof.
